(19) United States
(12) Patent Application Publication
(10) Pub. No.: US 2020/0039993 A1
(43) Pub. Date: Feb. 6, 2020

(54) COMPOUND FOR ORGANIC ELECTRONIC ELEMENT, ORGANIC ELECTRONIC ELEMENT USING SAME, AND ELECTRONIC DEVICE THEREFOR

(71) Applicant: DUK SAN NEOLUX CO., LTD, Cheonan-si, Chungcheongnam-do (KR)

(72) Inventors: Ga Eun LEE, Cheonan-si (KR); Hye Min CHO, Goyang-si (KR); Dae Hwan OH, Cheonan-si (KR); Seul Gi KIM, Daedeok-gu (KR); Jeong Seok KIM, Cheongju-si (KR)

(21) Appl. No.: 16/494,213

(22) PCT Filed: Mar. 9, 2018
(86) PCT No.: PCT/KR2018/002857

§ 371 Date: Sep. 13, 2019

(30) Foreign Application Priority Data
Mar. 16, 2017 (KR) 10-2017-0033368
Jan. 5, 2018 (KR) 10-2018-0001465

(51) Int. Cl. C07D 491/04; C07D 495/04; C07F 11/00; C07F 7/08; H01L 51/00

(52) U.S. Cl. CPC C07D 491/04; C07D 495/04; C07F 11/00; C07F 7/0816; H01L 51/0059; H01L 51/5056; H01L 51/0094; H01L 51/0067; H01L 51/0073; H01L 51/0074; H01L 51/0056

(57) ABSTRACT

The present disclosure provides: a compound capable of providing high light-emitting efficiency, low driving voltage, and improved lifetime of a device; an organic electronic element using the same; and an electronic device therefor.

### TECHNICAL FIELD

The present disclosure relates to a compound for an organic electric element, an organic electric element using same, and an electronic device using same.

### BACKGROUND ART

In general, organic light emission refers to a phenomenon in which electric energy is converted into light energy by using an organic material. An organic electric element utilizing organic light emission usually has a structure including an anode, a cathode, and an organic material layer interposed therebetween. In many cases, the organic material layer may have a multilayered structure including multiple layers made of different materials in order to improve efficiency and stability of an organic electric element, and for example, may be composed of a hole injection layer, a hole transport layer, a light emitting layer, an electron transport layer, an electron injection layer, and the like.

Materials used for an organic material layer in an organic electric element may be classified into light emitting materials and charge transport materials, for example, hole injection materials, hole transport materials, electron transport materials, electron injection materials, and the like, according to the function thereof.

Currently, the market for portable displays is on the way to large-area displays, and thus the sizes of displays are increasing. As a result, the larger power consumption than is required in existing portable displays is required. Therefore, the power consumption is a very important factor in portable displays with a limited power source, such as a battery, and efficiency and lifetime issue are also important factors to be solved.

Efficiency, lifetime, driving voltages, and the like are correlated with each other. If the efficiency is increased, the driving voltage is relatively lowered, and as the driving voltage is lowered, the crystallization of an organic material due to Joule heating generated during driving is reduced, and as a result, the lifetime shows a tendency to increase. However, the efficiency cannot be maximized only by simply improving organic material layers. The reason is that both long lifetime and high efficiency can be achieved when there is an optimal combination of energy levels, triplet excitation values (hereinafter, T1 values), inherent material properties (mobility, interfacial properties, etc.), and the like among the respective layers included in the organic material layer.

As for recent organic light emitting diodes, in order to solve the light emission problem in a hole transport layer, a light emitting auxiliary layer is necessarily present between the hole transport layer and a light emitting layer, and it is time to develop different light emitting auxiliary layers according to respective light emitting layers (R, G, B).

In general organic light emitting diodes, electrons are transferred from an electron transport layer to a light emitting layer and holes are transferred from a hole transport layer to the light emitting layer, so that the recombination of the electrons and the holes produces excitons.

However, a material used in the hole transport layer should have a low HOMO value, and thus it mainly has a low T1 value. As a result, the excitons produced from the light emitting layer are transported to the hole transport layer, resulting in a charge unbalance in the light emitting layer, thereby emitting light inside the hole transport layer or in the interface of the hole transport layer, causing a deterioration in color purity, a reduction in efficiency, and a low lifetime.

The use of a material having high hole mobility for a low driving voltage results in a decrease tendency in efficiency. The reason is that in general organic light emitting diodes, hole mobility is higher than electron mobility, resulting in a charge unbalance in the light emitting layer, causing a reduction in efficiency and a low lifetime.

Therefore, a light emitting auxiliary layer should be formed of a material having hole mobility (within a driving voltage range of a blue element of a full device), a high T1 (electron block) value, a wide bandgap for allowing a suitable driving voltage capable of solving a problem of the hole transport layer. However, this cannot be simply attained by structural characteristics of a core of a material for the light emitting auxiliary layer, and can be attained only under a combination of the core and sub-substituents of the material. Therefore, in order to improve efficiency and lifetime of an organic electric element, there is an urgent need to develop a material for a light emitting auxiliary layer, which has a high T1 value and a wide band gap.

That is, in order to allow the organic electric element to sufficiently exert excellent characteristics thereof, most of all, materials constituting an organic material layer in the element, for examples, a hole injection material, a hole transport material, a light emitting material, an electron transport material, an electron injection material, a material for a light emitting auxiliary layer, and the like should be supported by stable and efficient materials. However, the development of stable and efficient materials for the organic material layer for an organic electric element is not sufficiently achieved. Therefore, the development of new materials is continuously needed, and especially, the development of a material for the light emitting auxiliary layer and a material for the hole transport layer is urgently required.

### DETAILED DESCRIPTION OF THE INVENTION

### Technical Problem

In order to solve the above-mentioned problems occurring in the prior art, an object of the present disclosure is to provide a compound capable of lowering a driving voltage of an element and improving light emission efficiency and lifetime of the element, an organic electric element using same, and an electronic device using same.

### Technical Solution

In accordance with an aspect of the present disclosure, there is provided a compound represented by the formula below.

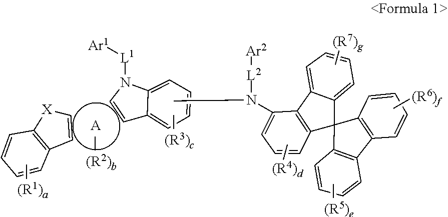

In another aspect of the present disclosure, there are provided an organic electric element using the compound represented by the above formula, and an electronic device using same.

### Advantageous Effects

By using the compound of the present disclosure, the driving voltage of an element can be lowered and the light emission efficiency and lifetime of the element can be greatly improved.

### BRIEF DESCRIPTION OF THE DRAWINGS

FIG. 1 is a cross-sectional view of an organic light emitting diode according to an embodiment of the present disclosure.

### MODE FOR CARRYING OUT THE INVENTION

Hereinafter, embodiments of the present disclosure will be described in detail with reference to the accompanying drawings.

In designation of reference numerals to components in each drawing, it should be noted that the same elements would be designated by the same reference numerals although they are shown in different drawings. Further, in the following description of the present disclosure, a detailed description of known functions and configurations incorporated herein will be omitted when it may make the subject matter of the present disclosure rather unclear.

Terms, such as first, second, A, B, (a), (b), or the like may be used herein when describing components of the present disclosure. Each of these terminologies is not used to define an essence, order, or sequence of a corresponding component but used merely to distinguish the corresponding component from other component(s). It should be noted that if it is described in the specification that one component is “connected,” “coupled” or “joined” to another component, a third component may be “connected,” “coupled,” and “joined” between the first and second components, although the first component may be directly connected, coupled or joined to the second component.

As used in the specification and the accompanying claims, unless otherwise stated, the meanings of the terms are as follows.

Unless otherwise stated, the term “halo” or “halogen” as used herein includes fluorine (F), bromine (Br), chlorine (Cl), or iodine (I).

Unless otherwise stated, the term “alkyl” or “alkyl group” as used herein refers to a radical of a saturated aliphatic functional group having 1 to 60 carbon atoms with single bond(s), including a straight-chain alkyl group, a branched-chain alkyl group, a cycloalkyl (alicyclic) group, an alkyl-substituted cycloalkyl group, and a cycloalkyl-substituted alkyl group.

Unless otherwise stated, the term “haloalkyl group” or “halogen alkyl group” as used herein refers to an alkyl group substituted with halogen.

The term “heteroalkyl group” as used herein refers to an alkyl group, of which at least one of carbon atoms is substituted with a heteroatom.

Unless otherwise stated, the term “alkenyl group” or “alkynyl group” as used herein refers to a functional group having 2 to 60 carbon atoms with a double or triple bond and including a straight-chain or branched-chain group, but is not limited thereto.

Unless otherwise stated, the term “cycloalkyl” as used herein refers to alkyl forming a ring having 3 to 60 carbon atoms, but is not limited thereto.

Unless otherwise stated, the term “alkoxyl group”, “alkoxy group”, or “alkyloxy group” as used herein refers to an alkyl group to which an oxygen radical is attached, the alkyl group having 1 to 60 carbon atoms, but is not limited thereto.

Unless otherwise stated, the term “alkenoxyl group”, “alkenoxy group”, “alkenyloxyl group”, or “alkenyloxy group” as used herein refers to an alkenyl group to which an oxygen radical is attached, the alkenyl group having 2 to 60 carbon atoms, but is not limited thereto.

Unless otherwise stated, the term “aryloxyl group” or “aryloxy group” as used herein refers to an aryl group to which an oxygen radical is attached to, the aryl group having 6 to 60 carbon atoms, but not limited thereto.

Unless otherwise stated, the terms “aryl group” and “arylene group” each as used herein refers to a functional group having 6 to 60 carbon atoms, but are not limited thereto. The aryl group or arylene group herein means to a monocyclic or polycyclic aromatic group, and includes an aromatic ring formed by adjacent substituents involved in linking or reaction. For example, the aryl group may be a phenyl group, a biphenyl group, a terphenyl group, a naphthyl group, an anthracenyl group, a fluorenyl group, a spiro-fluorene group, or a spiro-bifluorene group.

The prefix “aryl” or “ar” refers to a radical substituted with an aryl group. For example, an arylalkyl group is an alkyl group substituted with an aryl group and an arylalkenyl group is an alkenyl group substituted with an aryl group. A radical substituted with an aryl group has carbon atoms described herein.

When prefixes are named subsequently, it means that substituents are listed in the order described first. For example, an arylalkoxy group means an alkoxy group substituted with an aryl group; an alkoxylcarbonyl group means a carbonyl group substituted with an alkoxyl group; and an arylcarbonylalkenyl group means an alkenyl group substitutes with an arylcarbonyl group, wherein the arylcarbonyl group may be a carbonyl group substituted with an aryl group.

Unless otherwise stated, the term “heteroalkyl” as used herein refers to alkyl including at least one heteroatom. Unless otherwise stated, the term “heteroalkyl group” or “heteroarylene group” as used herein refers to an aryl group or arylene group having 2 to 60 carbon atoms and including at least one heteroatom, but is not limited thereto, and includes at least one of a monocyclic ring and a polycyclic ring, and may be formed by linkage of adjacent functional groups.

Unless otherwise stated, the term “heterocyclic group” as used herein refers to a functional group including at least one heteroatom, having 2 to 60 carbon atoms, including at least one of a monocyclic ring and a polycyclic ring. The heterocyclic group may be formed by linkage of adjacent functional groups.

Unless otherwise stated, the term “heteroatom” as used herein represents N, O, S, P, or Si.

Also, the “heterocyclic group” may include a ring containing SO₂ instead of carbon constituting a ring. For example, the “heterocyclic group” includes the compound below.

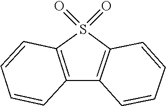 [SMILES: O=S1(=O)c2ccccc2-c2ccccc21]

Unless otherwise stated, the term “aliphatic” as used herein refers to an aliphatic hydrocarbon having 1 to 60 carbon atoms, and the term “aliphatic ring” refers to an aliphatic hydrocarbon ring having 3 to 60 carbon atoms.

Unless otherwise stated, the term “ring” as used herein includes an aliphatic ring having 3 to 60 carbon atoms, an aromatic group having 6 to 60 carbon atoms, a hetero ring having 2 to 60 carbon atoms, or a fusion ring composed of a combination thereof, and includes a saturated or unsaturated group

Besides the above-described hetero compounds, the other hetero compounds or hetero radicals include at least one heteroatom, but is not limited thereto.

Unless otherwise stated, the term “carbonyl” as used herein is represented by —COR′, wherein R′ may be hydrogen, an alkyl group having 1 to 20 carbon atoms, an aryl group having 6 to 30 carbon atoms, a cycloalkyl group having 3 to 30 carbon atoms, an alkenyl group having 2 to 20 carbon atoms, an alkynyl group having 2 to 20 carbon atoms, or a combination thereof.

Unless otherwise stated, the term “ether” as used herein is represented by —R—O—R′, wherein R or R′ each may be independently hydrogen, an alkyl group having 1 to 20 carbon atoms, an aryl group having 6 to 30 carbon atoms, a cycloalkyl group having 3 to 30 carbon atoms, an alkenyl group having 2 to 20 carbon atoms, an alkynyl group having 2 to 20 carbon atoms, or a combination thereof.

Unless otherwise stated, the term “substituted” in the term “substituted or unsubstituted” as used herein refers to a substitution with at least one substituent selected from the group consisting of deuterium, halogen, an amino group, a nitrile group, a nitro group, a C₁-C₂₀ alkyl group, a C₁-C₂₀ alkoxy group, a C₁-C₂₀ alkylamine group, a C₁-C₂₀ alkylthiophene group, a C₆-C₂₀ arylthiophene group, a C₂-C₂₀ alkenyl group, a C₂-C₂₀ alkynyl group, a C₃-C₂₀ cycloalkyl group, a C₆-C₆₀ aryl group, a C₆-C₂₀ aryl group substituted with deuterium, a C₈-C₂₀ arylalkenyl group, a silane group, a boron group, a germanium group, and a C₂-C₂₀ heterocyclic group.

Unless otherwise specified, the formulas used in the present disclosure are applied in the same manner as in the definition of substituents by the definition of an exponent in the formula below.

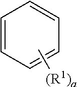

Here, when a is an integer of zero, substituent R¹ is absent; when a is an integer of 1, one substitutent R¹ is linked to any one of the carbon atoms constituting the benzene ring; and when a is an integer of 2 or 3, substituents R¹'s may be the same and different and may be linked to the benzene ring as follows. When a is an integer of 4 to 6, substituents R¹'s may be the same and different and may be linked to the benzene ring in a similar manner to that when a is an integer of 2 or 3. The indication of hydrogen atoms linked to carbon constituents of the benzene ring is omitted.

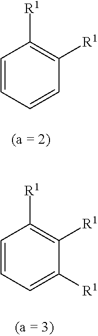

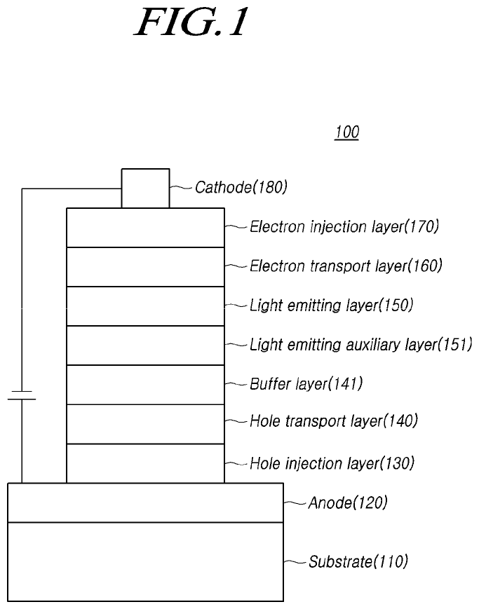

FIG. 1 is an exemplary view of an organic electric element according to an embodiment of the present disclosure.

Referring to FIG. 1, an organic electric element 100 according to the present disclosure includes a first electrode 120 formed on a substrate 110, a second electrode 180, and an organic material layer between the first electrode 120 and the second electrode 180, the organic material layer containing the compound according to the present disclosure. Here, the first electrode 120 may be an anode (positive electrode) and the second electrode 180 may be a cathode (negative electrode). In a case of an inverted organic electric element, the first electrode may be a cathode and the second electrode may be an anode.

The organic material layer may include a hole injection layer 130, a hole transport layer 140, a light emitting layer 150, an electron transport layer 160, and an electron injection layer 170, which are formed in sequence on the first electrode 120. Here, the other layers excluding the light emitting layer 150 may not be formed. The organic material layer may further include a hole blocking layer, an electron blocking layer, a light emitting auxiliary layer 151, an electron transport auxiliary layer, a buffer layer 141, or the like, and the electron transport layer 160 or the like may serve as a hole blocking layer.

Although not shown, the organic electric element according to the present disclosure may further include a protective layer or a light efficiency improving layer (capping layer), which is formed on one surface of at least one of the first and second electrodes, the surface being the opposite side to the organic material layer.

The compound according to the present disclosure employed in the organic material layer may be used as a host or dopant for the hole injection layer 130, the hole transport layer 140, the electron transport layer 160, the light emitting auxiliary layer 151, the electron transport auxiliary layer, the electron injection layer 170, or the light emitting layer 150, or a material for the light efficiency improvement layer. Preferably, the compound of the present disclosure may be used as a material for the hole transport layer and/or the light emitting auxiliary layer 151.

Since a band gap, electrical properties, interfacial properties, and the like may vary in spite of the same core depending on the type and position of a substituent to be attached, a selection of the core and a combination of sub-substituent attached to the core are also important. Specially, both long lifetime and high efficiency can be achieved when an optimal combination of energy levels, T₁ values, inherent material properties (mobility, interfacial properties, etc.), and the like among the respective layers included in the organic material layer is given.

Accordingly, energy levels, T₁ values, and inherent material properties (mobility, interfacial properties, etc.), and the like among the respective layers included in the organic material layer are optimized by forming the hole transport layer and/or the light emitting auxiliary layer 151 using the compound represented by Formula 1 of the present disclosure, so that both the lifetime and efficiency of an organic electric element can be improved.

An organic electric element according to an embodiment of the present disclosure may be manufactured using a physical vapor deposition (PVD) method. For example, the organic electric element may be manufactured by depositing a metal, a metal oxide having conductivity, or an alloy thereof, on the substrate to form the anode 120, forming the organic material layer including the hole injection layer 130, the hole transport layer 140, the light emitting layer 150, the electron transport layer 160, and the electron injection layer 170 thereon, and then depositing a material, which can be used for the cathode 180, thereon. The light emitting auxiliary layer 151 may be further formed between the hole transport layer 140 and the light emitting layer 150, and the electron transport auxiliary layer may be further formed between the light emitting layer 150 and the electron transport layer 160.

Also, the organic material layer may be manufactured to have a smaller number of layers using various polymer materials by, instead of a deposition method, a soluble process or solvent process, for example, a spin coating process, a nozzle printing process, an inkjet printing process, a slot coating process, a dip coating process, a roll-to-roll process, a doctor blading process, a screen printing process, or a thermal transfer method. Since the organic material layer according to the present disclosure may be formed in various ways, the scope of right of the present disclosure is not limited by a method of forming the organic material layer.

The organic electric element according to an embodiment of the present disclosure may be a top emission type, a bottom emission type, or a dual emission type, according to the used materials.

A white organic light emitting device (WOLED) facilitates the implementation of high resolution, has excellent processability, and has an advantage of being produced using conventional LCD color filter techniques. In this regard, various structures for WOLEDs, mainly used as back light units, have been suggested and patented. Representative WOLEDs are: a parallel side-by-side arrangement of red (R), green (G), and blue (B) light-emitting units on a mutual plane: a stacking arrangement of R, G, and B light emitting layers above and below; and a color conversion material (CCM) structure using electroluminescence by a blue (B) organic light emitting layer and photoluminescence from an inorganic fluorescent substance by using the light from the electroluminescence. The present disclosure can be applicable to such WOLEDs.

Further, the organic electric element according to an embodiment of the present disclosure may be any one of an organic light emitting diode (OLED), an organic solar cell, an organic photo conductor (OPC), an organic transistor (organic TFT), and an element for monochromatic or white illumination.

Another embodiment of the present disclosure provides an electronic device including: a display device, which includes the above-described organic electric element of the present disclosure; and a control unit for controlling the display device. Here, the electronic device may be a wired/wireless communication terminal, which is currently used or will be used in the future, and covers all kinds of electronic devices including a mobile communication terminal, such as a cellular phone, a personal digital assistant (PDA), an electronic dictionary, a point-to-multipoint (PMP), a remote controller, a navigation unit, a game player, various kinds of TVs, and various kinds of computers.

Hereinafter, a compound according to an aspect of the present disclosure will be described. A compound according to an aspect of the present disclosure is represented by Formula 1 below.

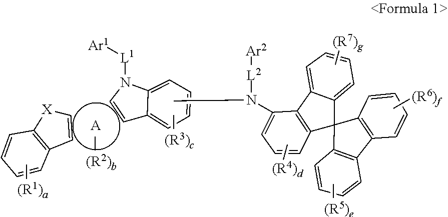

In Formula 1,

1) Ar¹ and Ar² each are independently the same as or different from each other, and selected from the group consisting of a C₆-C₆₀ aryl group, a fluorenyl group, a C₂-C₆₀ heterocyclic group containing at least one heteroatom of O, N, S, Si, and P, —N(R^{a})(R^{b}), a fused ring group of a C₆-C₆₀ aromatic ring and a C₃-C₆₀ aliphatic ring, a C₁-C₅₀ alkyl group, a C₂-C₂₀ alkenyl group, a C₂-C₂₀ alkynyl group, a C₁-C₃₀ alkoxy group, and a C₆-C₃₀ aryloxy group

(with the proviso that Ar¹ may not be a heteroaryl containing N);

2) X is any one of N-L³-Ar³, O, S, Se, Ge, and SiR^{c}R^{d};

3) R¹ to R⁷ each are independently the same as or different from each other, and selected from the group consisting of deuterium, tritium, halogen, a cyano group, a nitro group, a C₆-C₆₀ aryl group, a fluorenyl group, a C₂-C₆₀ heterocyclic group containing at least one heteroatom selected from the group consisting of O, N, S, Si, and P, a fused ring group of a C₃-C₆₀ aliphatic ring and a C₆-C₆₀ aromatic ring, a C₁-C₅₀ alkyl group, a C₂-C₂₀ alkenyl group, a C₂-C₂₀ alkynyl group, a C₁-C₃₀ alkoxy group, and a C₆-C₃₀ aryloxy group, wherein R¹ to R⁷ may bind to each other to form a ring

(in the presence of a plurality of R¹'s to R⁷'s, at least one pair of independently neighboring R¹'s, R²'s, R³'s, R⁴'s, R⁵'s, R⁶'s, and R⁷'s may bind to each other to form a ring, provided that R¹'s to R⁷'s forming no ring are the same as defined above);

4) a, e, f, and g are an integer of 0 to 4, b is an integer of 0 to 2, and d is an integer of 0 to 3;

5) ring A is a C₆ aryl group;

6) L¹ to L³ each are selected from the group consisting of a direct bond, a C₆-C₆₀ arylene group, a C₂-C₆₀ heterocyclic group containing at least one heteroatom selected from the group consisting of O, N, S, Si, and P, a fluorenylene group, a divalent fused ring of a C₃-C₆₀ aliphatic ring and a C₆-C₆₀ aromatic ring, and a C₁-C₆₀ aliphatic hydrocarbon group;

7) Ar³ is selected from the group consisting of a C₆-C₆₀ aryl group, a fluorenyl group, a C₂-C₆₀ heterocyclic group containing at least one heteroatom selected from the group consisting of O, N, S, Si, and P, —N(R^{a})(R^{b}), a fused ring group of a C₆-C₆₀ aromatic ring and a C₃-C₆₀ aliphatic ring, a C₁-C₅₀ alkyl group, a C₂-C₂₀ alkenyl group, a C₂-C₂₀ alkynyl group, a C₁-C₃₀ alkoxy group, and a C₆-C₃₀ aryloxy group; and

8) R^{a}, R^{b}, R^{c}, and R^{d} each are independently selected from the group consisting of deuterium, tritium, halogen, a cyano group, a nitro group, a C₆-C₆₀ aryl group, a fluorenyl group, a C₂-C₆₀ heterocyclic group containing at least one heteroatom selected from the group consisting of O, N, S, Si, and P, a fused ring group of a C₃-C₆₀ aliphatic ring and a C₆-C₆₀ aromatic ring, a C₁-C₅₀ alkyl group, a C₂-C₂₀ alkenyl group, a C₂-C₂₀ alkynyl group, a C₁-C₃₀ alkoxy group, and a C₆-C₃₀ aryloxy group

(wherein R^{c} and R^{d} may form a spiro compound by forming a ring),

wherein the aryl group, fluorenylene group, fluorenyl group, heterocyclic group, alkyl group, fused ring group, alkenyl group, alkoxy group, and aryloxy group each may be further substituted with at least one substituent selected from the group consisting of deuterium, halogen, a silane group substituted or unsubstituted with a C₁-C₂₀ alkyl group or a C₆-C₂₀ aryl group, a siloxane group, a boron group, a germanium group, a cyano group, a nitro group, —N(R^{e})(R^{f}) (here, R^{e} and R^{f} are the same as the above-described definition of R^{a} and R^{d}, respectively), a C₁-C₂₀ alkylthio group, a C₁-C₂₀ alkoxy group, a C₁-C₂₀ alkyl group, a C₂-C₂₀ alkenyl group, a C₂-C₂₀ alkynyl group, a C₆-C₂₀ aryl group, a C₆-C₂₀ aryl group substituted with deuterium, a fluorenyl group, a C₂-C₂₀ heterocyclic group containing at least one heteroatom selected from the group consisting of O, N, S, Si, and P, a C₃-C₂₀ cycloalkyl group, a C₇-C₂₀ arylalkyl group, and a C₈-C₂₀ arylalkenyl group, and when those substituents are adjacent, the substituents may bind to each other to form a ring.

In addition, these substituents may bind to each other to from a ring, wherein the “ring” refers to a fusion ring composed of an aliphatic ring having 3 to 60 carbon atoms, an aromatic group having 6 to 60 carbon atoms, or a heterocyclic group having 2 to 60 carbon atoms, or a combination thereof, and includes a saturated or unsaturated ring.

Here, the aryl group may be an aryl group having 6-60 carbon atoms, preferably 6-40 carbon atoms, and more preferably 6-30 carbon atoms; the heterocyclic group may be a heterocyclic group having 2-60 carbon atoms, preferably 2-30 carbon atoms, and more preferably 2-20 carbon atoms; and the alkyl group may be an alkyl group having 1-50 carbon atoms, preferably 1-30 carbon atoms, more preferably 1-20 carbon atoms, and especially preferably 1-10 carbon atoms.

In the above-described aryl or arylene group, the aryl or arylene group may be independently a phenyl group, a biphenyl group, a terphenyl group, a naphthyl group, or a phenanthryl group, or a phenylene group, a biphenylene group, a terphenylene group, a naphthylene group, or a phenanthrylene group.

More specifically, the compound represented by Formula 1 may be one of the compounds below, and is not limited to only the compounds below.

Formula 1 above may be represented by one of Formulas 2 to 7.

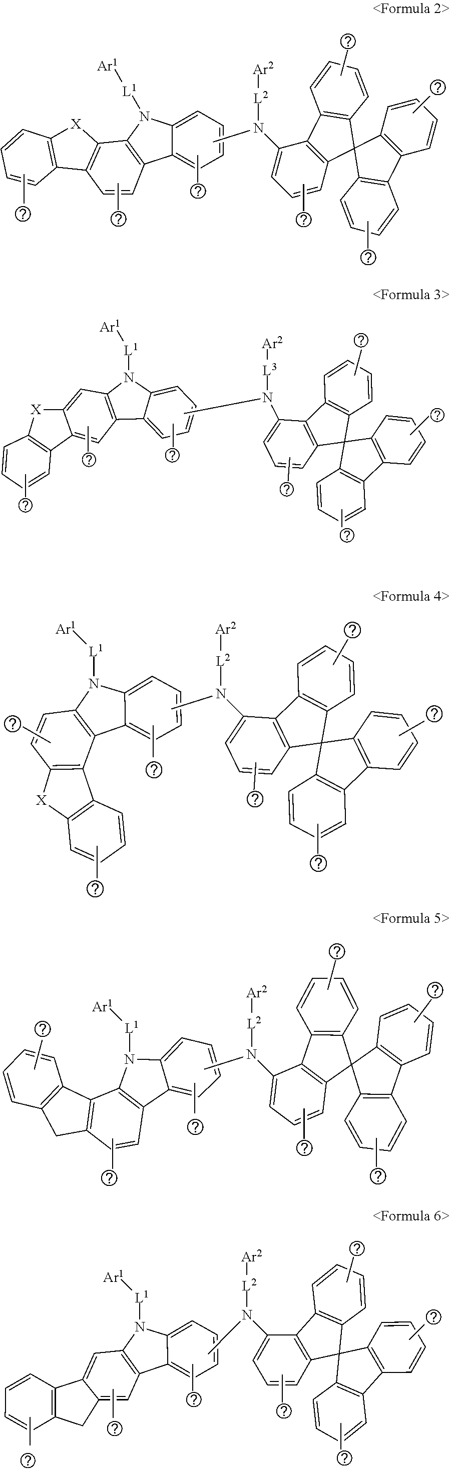

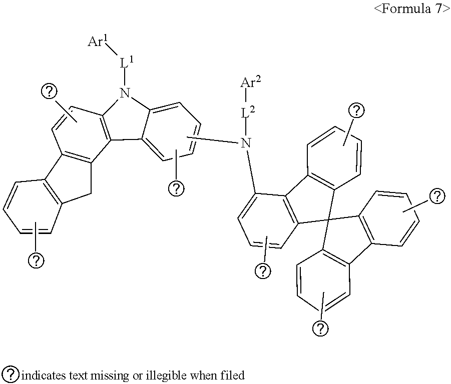

In Formulas 2 to 7,

X, L¹, L², Ar¹, Ar², R¹ to R⁷, and a to f are the same as X, L¹, L², Ar¹, Ar², R¹ to R⁷, and a to f defined in Formula 1 above, respectively.

More specifically, the compound represented by Formula 1 may be any one of the compounds below, and is not limited to only the compounds below:

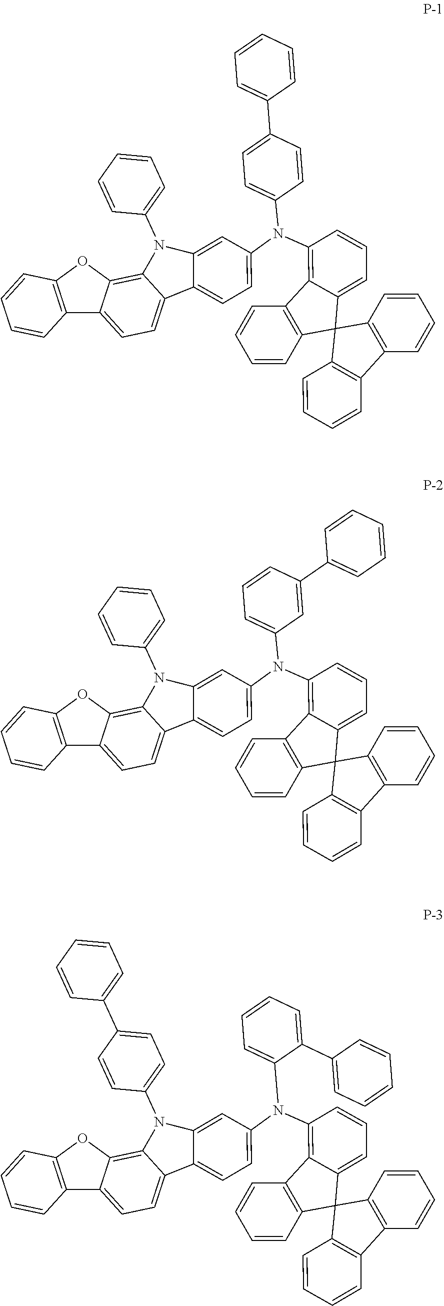 [SMILES: c1ccc(-c2ccc(N(c3ccc4c5ccc6c7ccccc7oc6c5n(-c5ccccc5)c4c3)c3cccc4c3-c3ccccc3C43c4ccccc4-c4ccccc43)cc2)cc1 ; c1ccc(-c2cccc(N(c3ccc4c5ccc6c7ccccc7oc6c5n(-c5ccccc5)c4c3)c3cccc4c3-c3ccccc3C43c4ccccc4-c4ccccc43)c2)cc1 ; c1ccc(-c2ccc(-n3c4cc(N(c5ccccc5-c5ccccc5)c5cccc6c5-c5ccccc5C65c6ccccc6-c6ccccc65)ccc4c4ccc5c6ccccc6oc5c43)cc2)cc1]

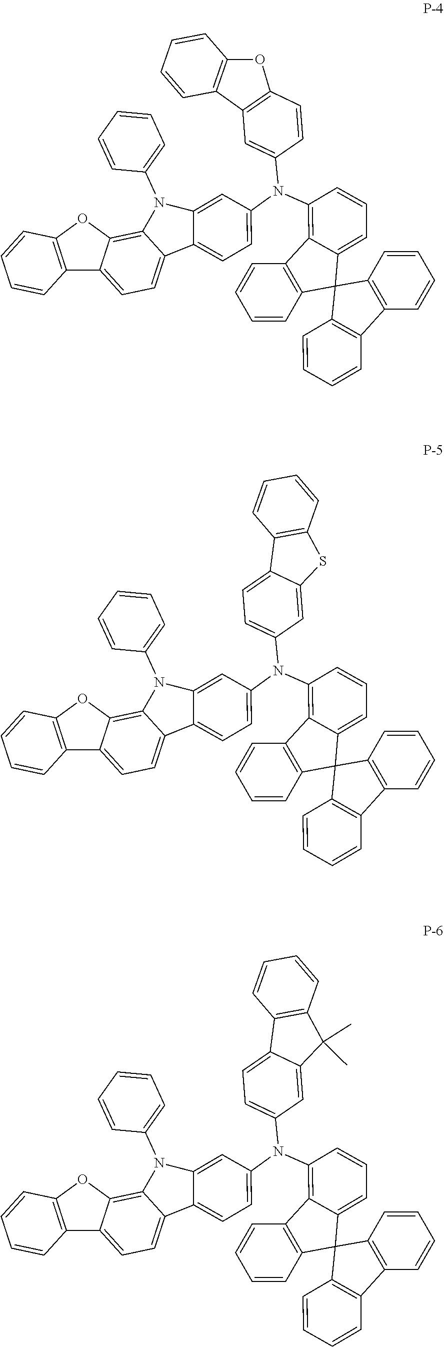 [SMILES: c1ccc(-n2c3cc(N(c4ccc5oc6ccccc6c5c4)c4cccc5c4-c4ccccc4C54c5ccccc5-c5ccccc54)ccc3c3ccc4c5ccccc5oc4c32)cc1 ; c1ccc(-n2c3cc(N(c4ccc5c(c4)sc4ccccc45)c4cccc5c4-c4ccccc4C54c5ccccc5-c5ccccc54)ccc3c3ccc4c5ccccc5oc4c32)cc1 ; CC1(C)c2ccccc2-c2ccc(N(c3ccc4c5ccc6c7ccccc7oc6c5n(-c5ccccc5)c4c3)c3cccc4c3-c3ccccc3C43c4ccccc4-c4ccccc43)cc21]

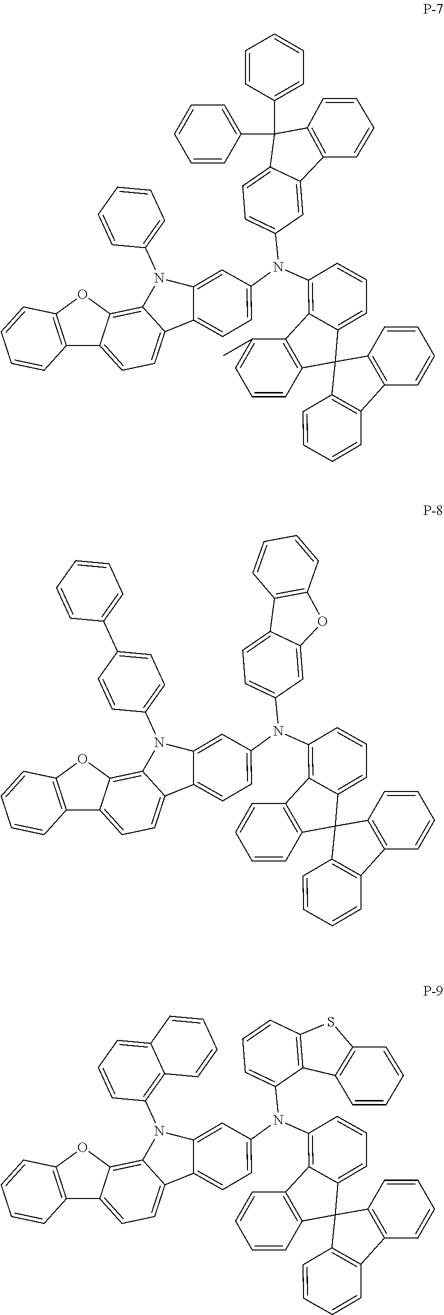 [SMILES: Cc1cccc2c1-c1c(N(c3ccc4c(c3)-c3ccccc3C4(c3ccccc3)c3ccccc3)c3ccc4c5ccc6c7ccccc7oc6c5n(-c5ccccc5)c4c3)cccc1C21c2ccccc2-c2ccccc21 ; c1ccc(-c2ccc(-n3c4cc(N(c5ccc6c(c5)oc5ccccc56)c5cccc6c5-c5ccccc5C65c6ccccc6-c6ccccc65)ccc4c4ccc5c6ccccc6oc5c43)cc2)cc1 ; c1ccc2c(c1)-c1ccccc1C21c2ccccc2-c2c(N(c3ccc4c5ccc6c7ccccc7oc6c5n(-c5cccc6ccccc56)c4c3)c3cccc4sc5ccccc5c34)cccc21]

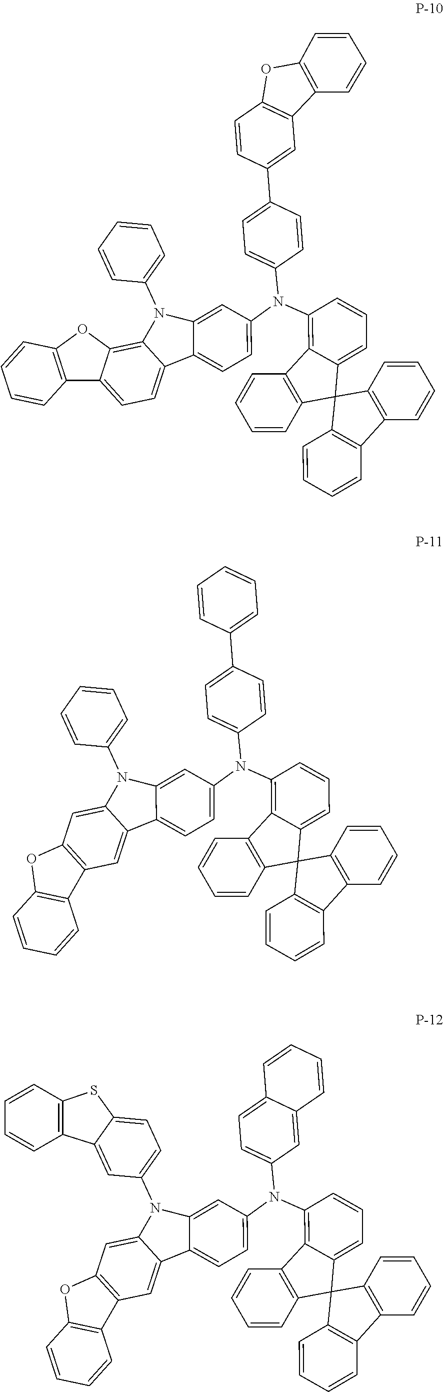 [SMILES: c1ccc(-n2c3cc(N(c4ccc(-c5ccc6oc7ccccc7c6c5)cc4)c4cccc5c4-c4ccccc4C54c5ccccc5-c5ccccc54)ccc3c3ccc4c5ccccc5oc4c32)cc1 ; c1ccc(-c2ccc(N(c3ccc4c5cc6c(cc5n(-c5ccccc5)c4c3)oc3ccccc36)c3cccc4c3-c3ccccc3C43c4ccccc4-c4ccccc43)cc2)cc1 ; c1ccc2c(c1)-c1ccccc1C21c2ccccc2-c2c(N(c3ccc4ccccc4c3)c3ccc4c5cc6c(cc5n(-c5ccc7sc8ccccc8c7c5)c4c3)oc3ccccc36)cccc21]

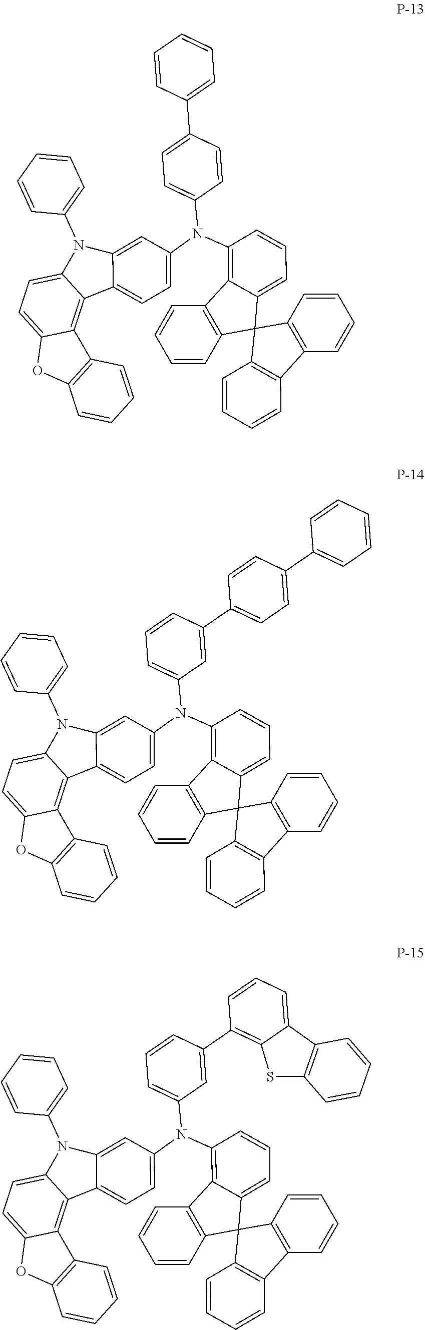 [SMILES: c1ccc(-c2ccc(N(c3ccc4c5c6c(ccc5n(-c5ccccc5)c4c3)oc3ccccc36)c3cccc4c3-c3ccccc3C43c4ccccc4-c4ccccc43)cc2)cc1 ; c1ccc(-c2ccc(-c3cccc(N(c4ccc5c6c7c(ccc6n(-c6ccccc6)c5c4)oc4ccccc47)c4cccc5c4-c4ccccc4C54c5ccccc5-c5ccccc54)c3)cc2)cc1 ; c1ccc(-n2c3cc(N(c4cccc(-c5cccc6c5sc5ccccc56)c4)c4cccc5c4-c4ccccc4C54c5ccccc5-c5ccccc54)ccc3c3c4c(ccc32)oc2ccccc24)cc1]

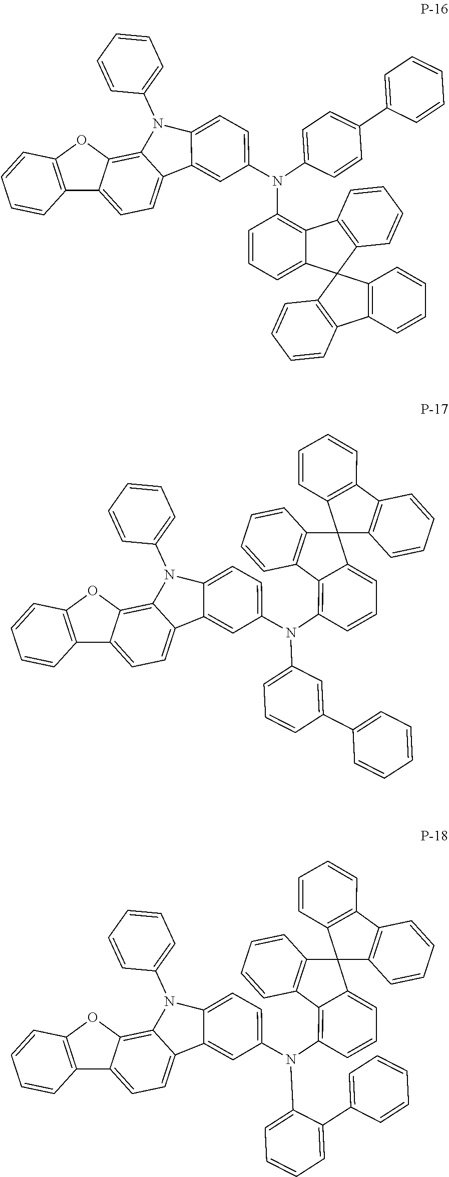 [SMILES: c1ccc(-c2ccc(N(c3ccc4c(c3)c3ccc5c6ccccc6oc5c3n4-c3ccccc3)c3cccc4c3-c3ccccc3C43c4ccccc4-c4ccccc43)cc2)cc1 ; c1ccc(-c2cccc(N(c3ccc4c(c3)c3ccc5c6ccccc6oc5c3n4-c3ccccc3)c3cccc4c3-c3ccccc3C43c4ccccc4-c4ccccc43)c2)cc1 ; c1ccc(-c2ccccc2N(c2ccc3c(c2)c2ccc4c5ccccc5oc4c2n3-c2ccccc2)c2cccc3c2-c2ccccc2C32c3ccccc3-c3ccccc32)cc1]

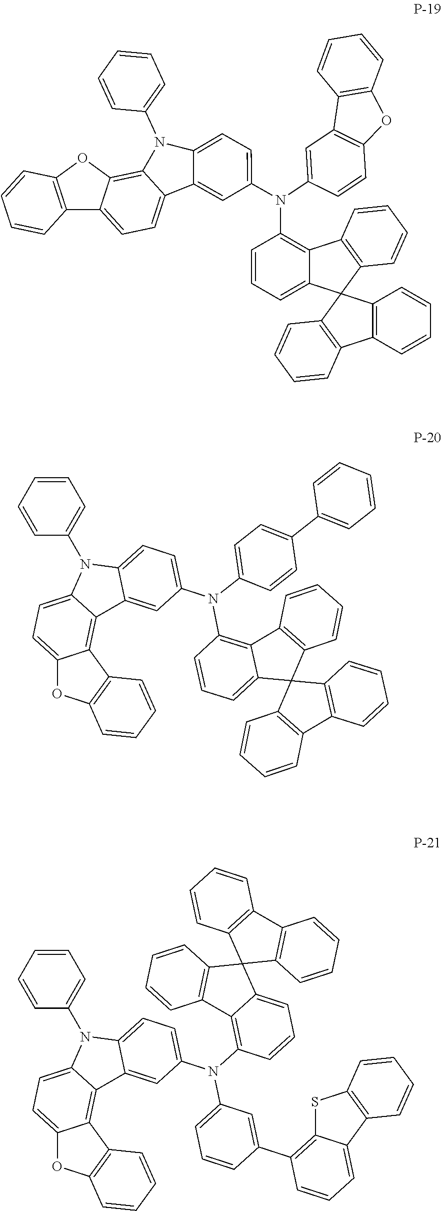 [SMILES: c1ccc(-n2c3ccc(N(c4ccc5oc6ccccc6c5c4)c4cccc5c4-c4ccccc4C54c5ccccc5-c5ccccc54)cc3c3ccc4c5ccccc5oc4c32)cc1 ; c1ccc(-c2ccc(N(c3ccc4c(c3)c3c5c(ccc3n4-c3ccccc3)oc3ccccc35)c3cccc4c3-c3ccccc3C43c4ccccc4-c4ccccc43)cc2)cc1 ; c1ccc(-n2c3ccc(N(c4cccc(-c5cccc6c5sc5ccccc56)c4)c4cccc5c4-c4ccccc4C54c5ccccc5-c5ccccc54)cc3c3c4c(ccc32)oc2ccccc24)cc1]

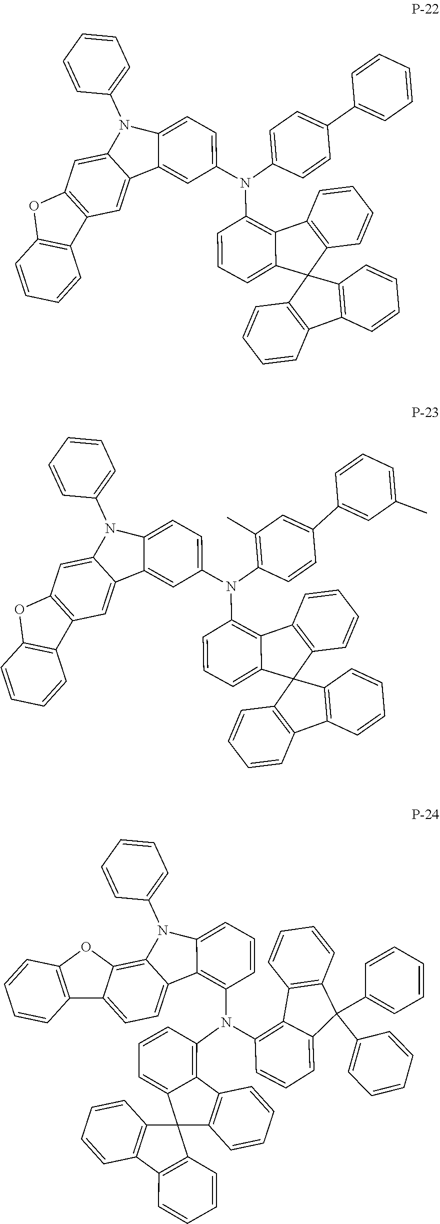 [SMILES: c1ccc(-c2ccc(N(c3ccc4c(c3)c3cc5c(cc3n4-c3ccccc3)oc3ccccc35)c3cccc4c3-c3ccccc3C43c4ccccc4-c4ccccc43)cc2)cc1 ; Cc1cccc(-c2ccc(N(c3ccc4c(c3)c3cc5c(cc3n4-c3ccccc3)oc3ccccc35)c3cccc4c3-c3ccccc3C43c4ccccc4-c4ccccc43)c(C)c2)c1 ; c1ccc(-n2c3cccc(N(c4cccc5c4-c4ccccc4C5(c4ccccc4)c4ccccc4)c4cccc5c4-c4ccccc4C54c5ccccc5-c5ccccc54)c3c3ccc4c5ccccc5oc4c32)cc1]

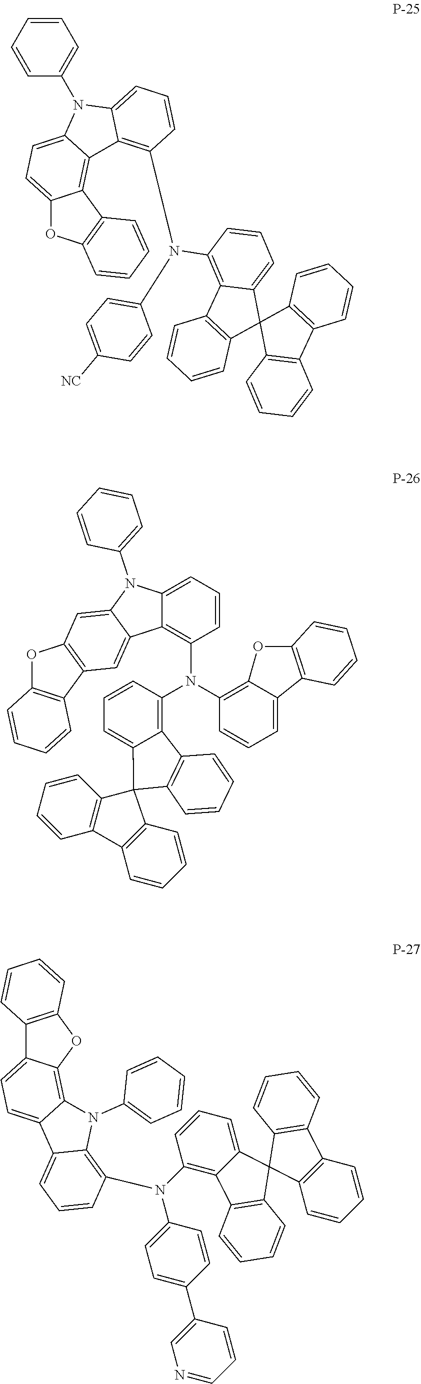 [SMILES: N#Cc1ccc(N(c2cccc3c2-c2ccccc2C32c3ccccc3-c3ccccc32)c2cccc3c2c2c4c(ccc2n3-c2ccccc2)oc2ccccc24)cc1 ; c1ccc(-n2c3cc4oc5ccccc5c4cc3c3c(N(c4cccc5c4-c4ccccc4C54c5ccccc5-c5ccccc54)c4cccc5c4oc4ccccc45)cccc32)cc1 ; c1ccc(-n2c3c(N(c4ccc(-c5cccnc5)cc4)c4cccc5c4-c4ccccc4C54c5ccccc5-c5ccccc54)cccc3c3ccc4c5ccccc5oc4c32)cc1]

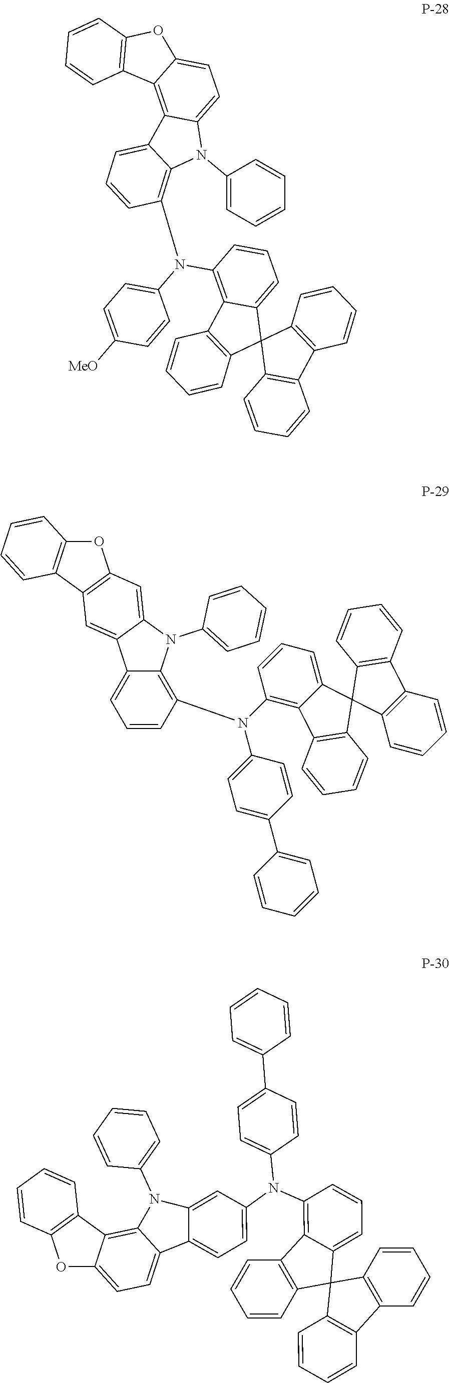 [SMILES: COc1ccc(N(c2cccc3c2-c2ccccc2C32c3ccccc3-c3ccccc32)c2cccc3c4c5c(ccc4n(-c4ccccc4)c23)oc2ccccc25)cc1 ; c1ccc(-c2ccc(N(c3cccc4c3-c3ccccc3C43c4ccccc4-c4ccccc43)c3cccc4c5cc6c(cc5n(-c5ccccc5)c34)oc3ccccc36)cc2)cc1 ; c1ccc(-c2ccc(N(c3ccc4c5ccc6oc7ccccc7c6c5n(-c5ccccc5)c4c3)c3cccc4c3-c3ccccc3C43c4ccccc4-c4ccccc43)cc2)cc1]

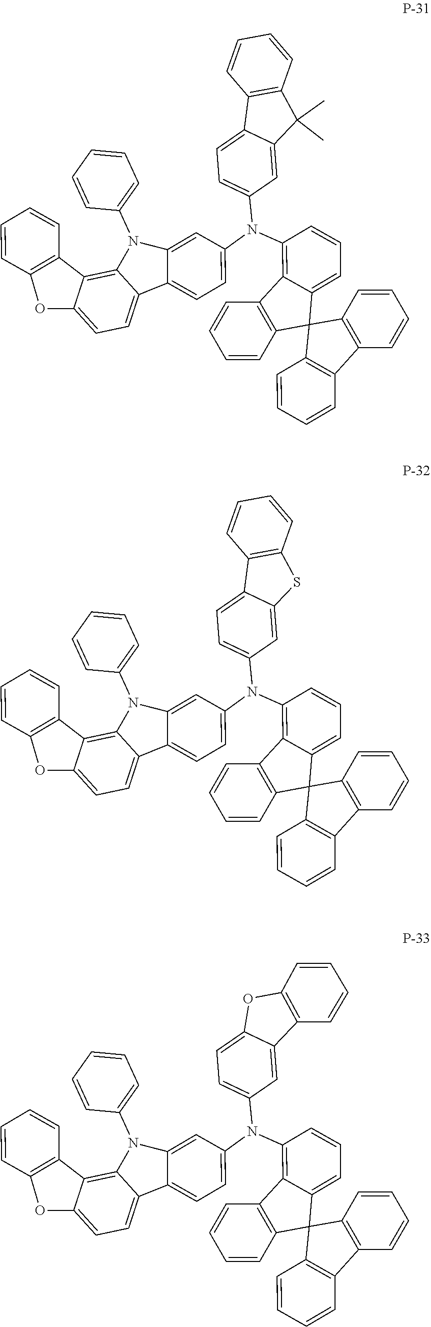 [SMILES: CC1(C)c2ccccc2-c2ccc(N(c3ccc4c5ccc6oc7ccccc7c6c5n(-c5ccccc5)c4c3)c3cccc4c3-c3ccccc3C43c4ccccc4-c4ccccc43)cc21 ; c1ccc(-n2c3cc(N(c4ccc5c(c4)sc4ccccc45)c4cccc5c4-c4ccccc4C54c5ccccc5-c5ccccc54)ccc3c3ccc4oc5ccccc5c4c32)cc1 ; c1ccc(-n2c3cc(N(c4ccc5oc6ccccc6c5c4)c4cccc5c4-c4ccccc4C54c5ccccc5-c5ccccc54)ccc3c3ccc4oc5ccccc5c4c32)cc1]

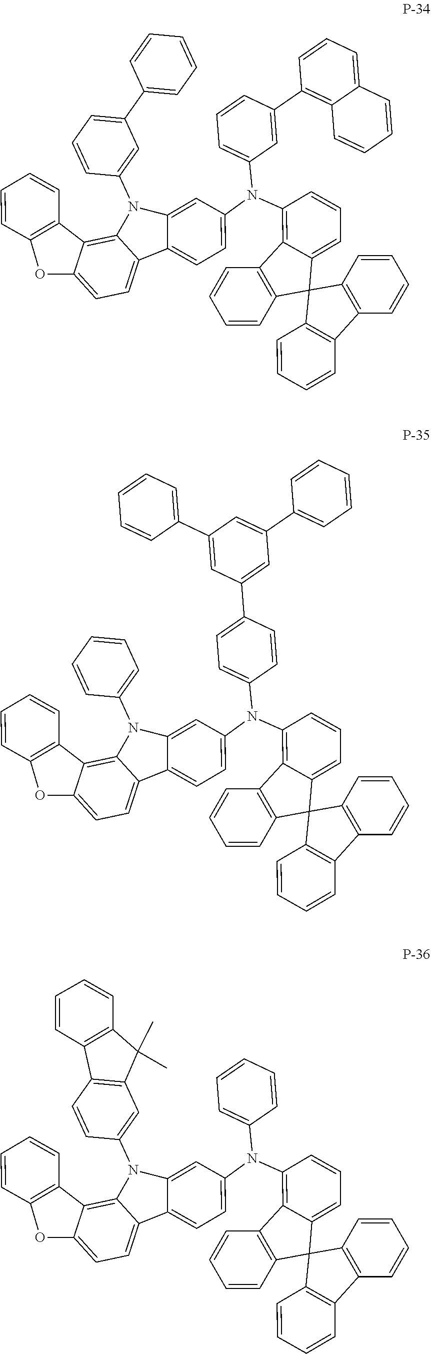 [SMILES: c1ccc(-c2cccc(-n3c4cc(N(c5cccc(-c6cccc7ccccc67)c5)c5cccc6c5-c5ccccc5C65c6ccccc6-c6ccccc65)ccc4c4ccc5oc6ccccc6c5c43)c2)cc1 ; c1ccc(-c2cc(-c3ccccc3)cc(-c3ccc(N(c4ccc5c6ccc7oc8ccccc8c7c6n(-c6ccccc6)c5c4)c4cccc5c4-c4ccccc4C54c5ccccc5-c5ccccc54)cc3)c2)cc1 ; CC1(C)c2ccccc2-c2ccc(-n3c4cc(N(c5ccccc5)c5cccc6c5-c5ccccc5C65c6ccccc6-c6ccccc65)ccc4c4ccc5oc6ccccc6c5c43)cc21]

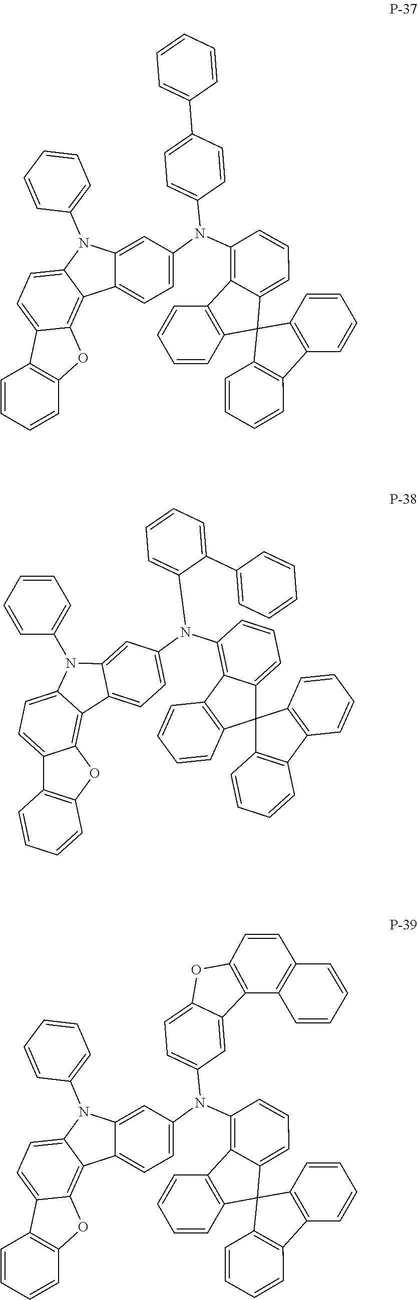 [SMILES: c1ccc(-c2ccc(N(c3ccc4c5c6oc7ccccc7c6ccc5n(-c5ccccc5)c4c3)c3cccc4c3-c3ccccc3C43c4ccccc4-c4ccccc43)cc2)cc1 ; c1ccc(-c2ccccc2N(c2ccc3c4c5oc6ccccc6c5ccc4n(-c4ccccc4)c3c2)c2cccc3c2-c2ccccc2C32c3ccccc3-c3ccccc32)cc1 ; c1ccc(-n2c3cc(N(c4ccc5oc6ccc7ccccc7c6c5c4)c4cccc5c4-c4ccccc4C54c5ccccc5-c5ccccc54)ccc3c3c4oc5ccccc5c4ccc32)cc1]

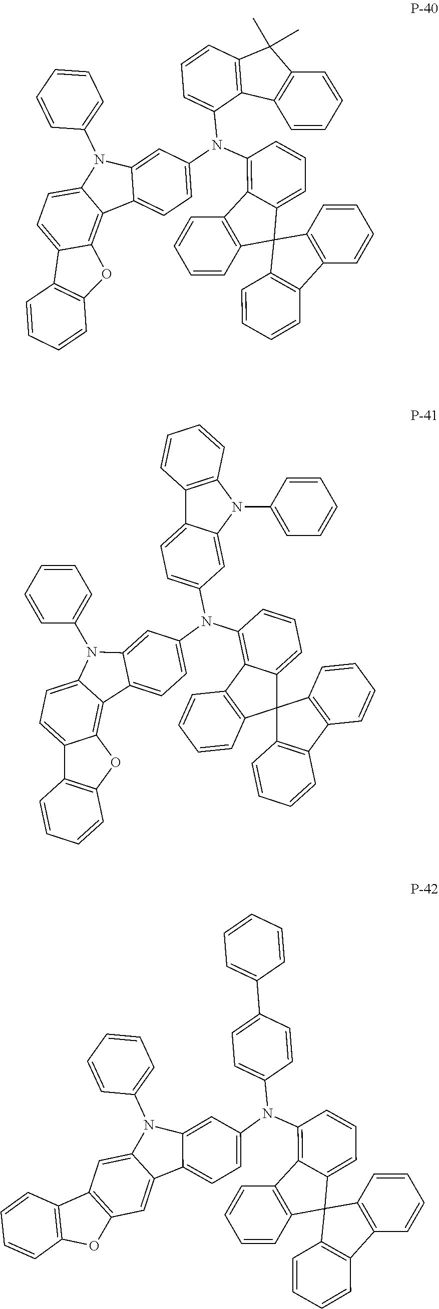 [SMILES: CC1(C)c2ccccc2-c2c(N(c3ccc4c5c6oc7ccccc7c6ccc5n(-c5ccccc5)c4c3)c3cccc4c3-c3ccccc3C43c4ccccc4-c4ccccc43)cccc21 ; c1ccc(-n2c3ccccc3c3ccc(N(c4ccc5c6c7oc8ccccc8c7ccc6n(-c6ccccc6)c5c4)c4cccc5c4-c4ccccc4C54c5ccccc5-c5ccccc54)cc32)cc1 ; c1ccc(-c2ccc(N(c3ccc4c5cc6oc7ccccc7c6cc5n(-c5ccccc5)c4c3)c3cccc4c3-c3ccccc3C43c4ccccc4-c4ccccc43)cc2)cc1]

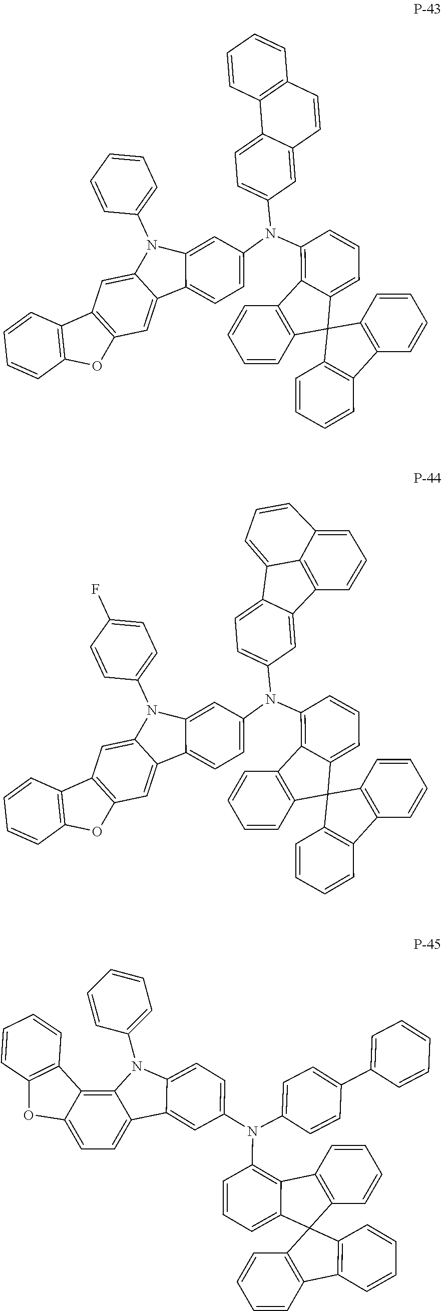 [SMILES: c1ccc(-n2c3cc(N(c4ccc5c(ccc6ccccc65)c4)c4cccc5c4-c4ccccc4C54c5ccccc5-c5ccccc54)ccc3c3cc4oc5ccccc5c4cc32)cc1 ; Fc1ccc(-n2c3cc(N(c4ccc5c(c4)-c4cccc6cccc-5c46)c4cccc5c4-c4ccccc4C54c5ccccc5-c5ccccc54)ccc3c3cc4oc5ccccc5c4cc32)cc1 ; c1ccc(-c2ccc(N(c3ccc4c(c3)c3ccc5oc6ccccc6c5c3n4-c3ccccc3)c3cccc4c3-c3ccccc3C43c4ccccc4-c4ccccc43)cc2)cc1]

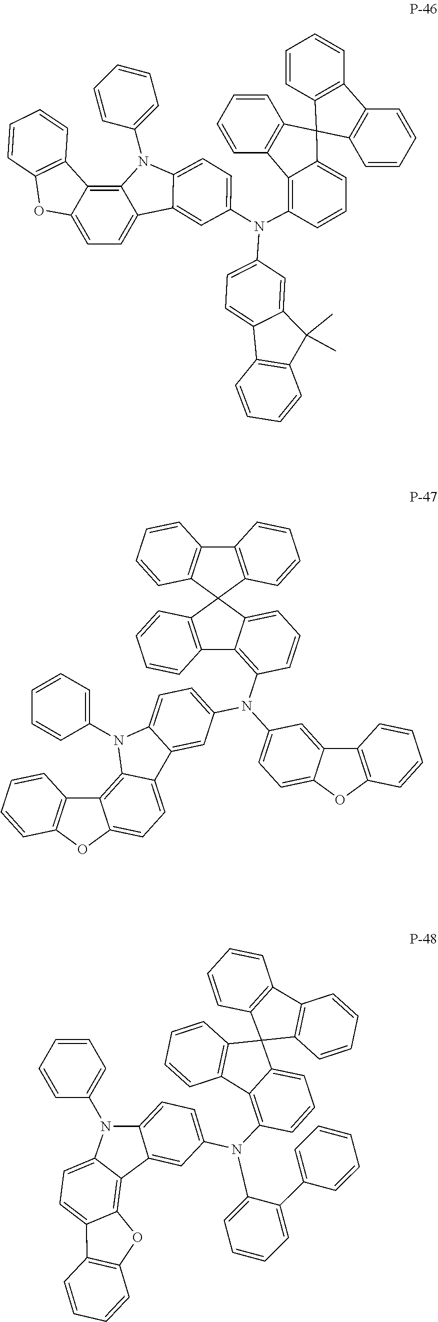 [SMILES: CC1(C)c2ccccc2-c2ccc(N(c3ccc4c(c3)c3ccc5oc6ccccc6c5c3n4-c3ccccc3)c3cccc4c3-c3ccccc3C43c4ccccc4-c4ccccc43)cc21 ; c1ccc(-n2c3ccc(N(c4ccc5oc6ccccc6c5c4)c4cccc5c4-c4ccccc4C54c5ccccc5-c5ccccc54)cc3c3ccc4oc5ccccc5c4c32)cc1 ; c1ccc(-c2ccccc2N(c2ccc3c(c2)c2c4oc5ccccc5c4ccc2n3-c2ccccc2)c2cccc3c2-c2ccccc2C32c3ccccc3-c3ccccc32)cc1]

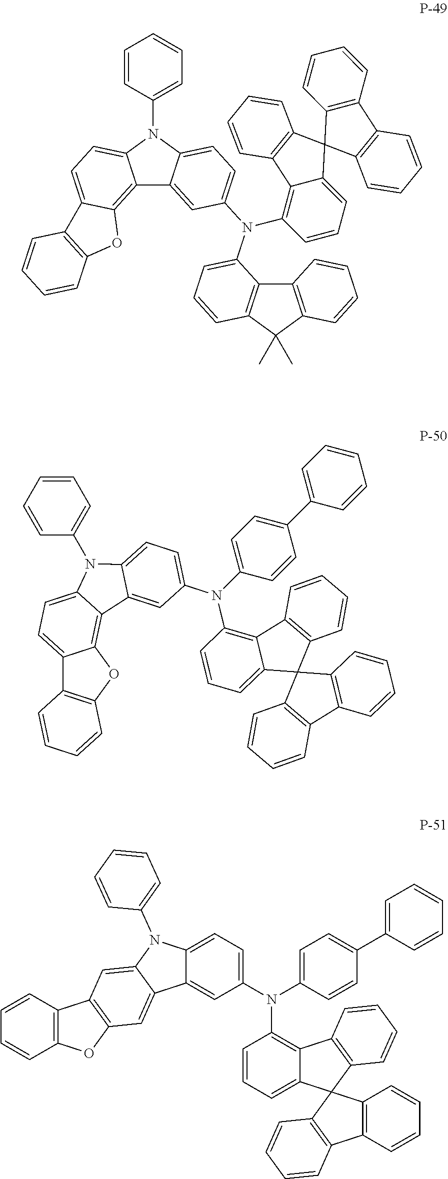 [SMILES: CC1(C)c2ccccc2-c2c(N(c3ccc4c(c3)c3c5oc6ccccc6c5ccc3n4-c3ccccc3)c3cccc4c3-c3ccccc3C43c4ccccc4-c4ccccc43)cccc21 ; c1ccc(-c2ccc(N(c3ccc4c(c3)c3c5oc6ccccc6c5ccc3n4-c3ccccc3)c3cccc4c3-c3ccccc3C43c4ccccc4-c4ccccc43)cc2)cc1 ; c1ccc(-c2ccc(N(c3ccc4c(c3)c3cc5oc6ccccc6c5cc3n4-c3ccccc3)c3cccc4c3-c3ccccc3C43c4ccccc4-c4ccccc43)cc2)cc1]

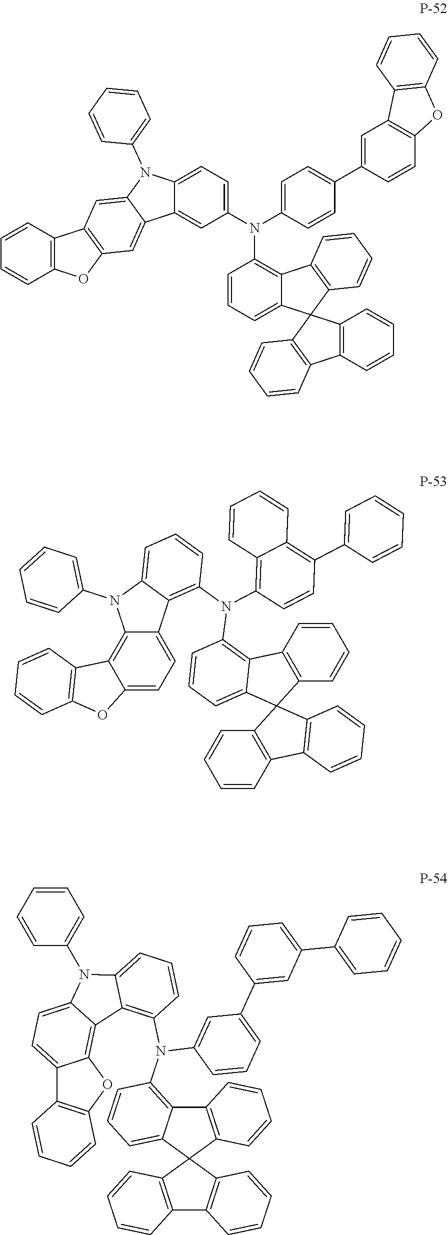 [SMILES: c1ccc(-n2c3ccc(N(c4ccc(-c5ccc6oc7ccccc7c6c5)cc4)c4cccc5c4-c4ccccc4C54c5ccccc5-c5ccccc54)cc3c3cc4oc5ccccc5c4cc32)cc1 ; c1ccc(-c2ccc(N(c3cccc4c3-c3ccccc3C43c4ccccc4-c4ccccc43)c3cccc4c3c3ccc5oc6ccccc6c5c3n4-c3ccccc3)c3ccccc23)cc1 ; c1ccc(-c2cccc(-c3cccc(N(c4cccc5c4-c4ccccc4C54c5ccccc5-c5ccccc54)c4cccc5c4c4c6oc7ccccc7c6ccc4n5-c4ccccc4)c3)c2)cc1]

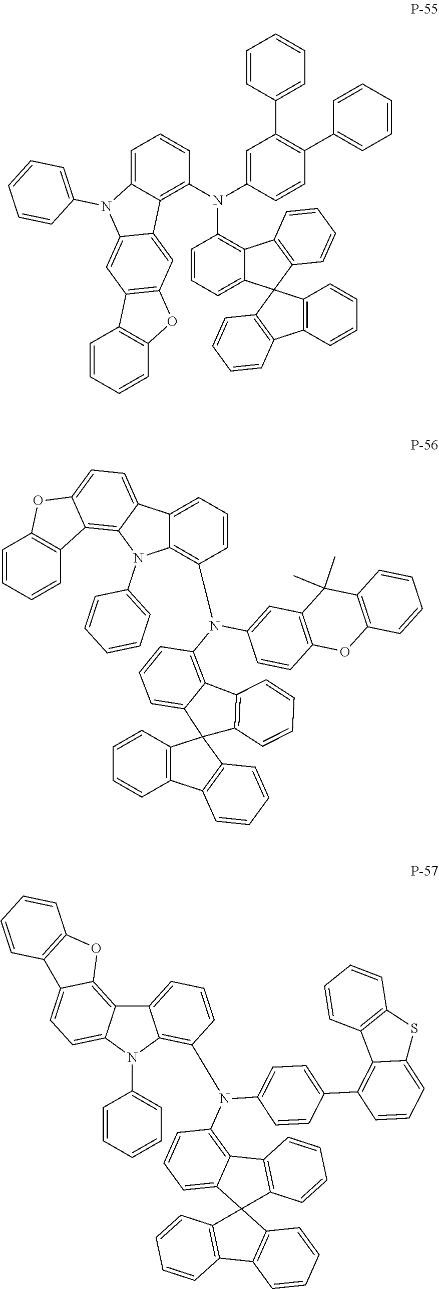 [SMILES: c1ccc(-c2ccc(N(c3cccc4c3-c3ccccc3C43c4ccccc4-c4ccccc43)c3cccc4c3c3cc5oc6ccccc6c5cc3n4-c3ccccc3)cc2-c2ccccc2)cc1 ; CC1(C)c2ccccc2Oc2ccc(N(c3cccc4c3-c3ccccc3C43c4ccccc4-c4ccccc43)c3cccc4c5ccc6oc7ccccc7c6c5n(-c5ccccc5)c34)cc21 ; c1ccc(-n2c3ccc4c5ccccc5oc4c3c3cccc(N(c4ccc(-c5cccc6sc7ccccc7c56)cc4)c4cccc5c4-c4ccccc4C54c5ccccc5-c5ccccc54)c32)cc1]

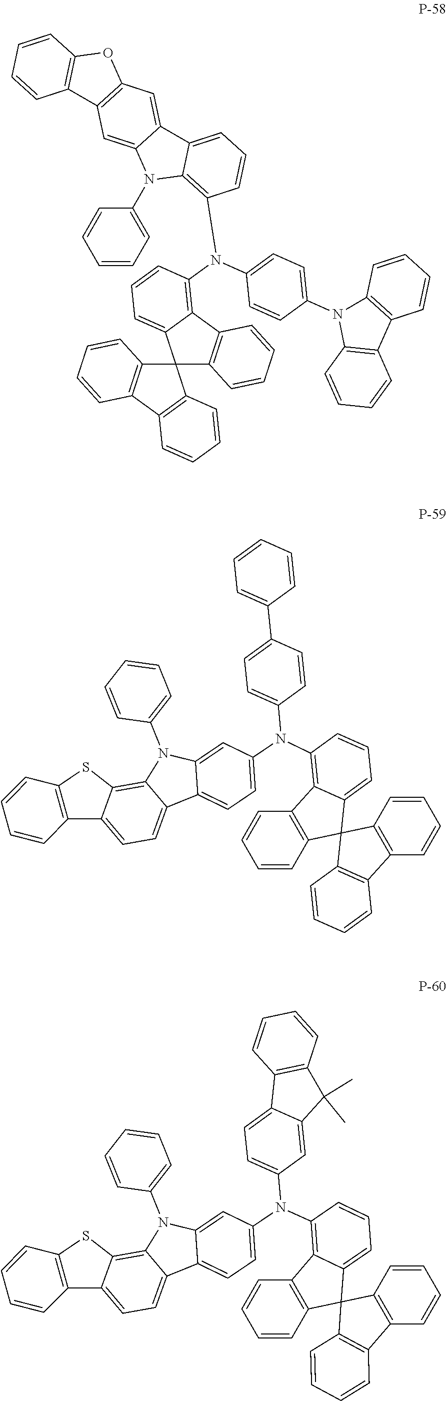 [SMILES: c1ccc(-n2c3cc4c(cc3c3cccc(N(c5ccc(-n6c7ccccc7c7ccccc76)cc5)c5cccc6c5-c5ccccc5C65c6ccccc6-c6ccccc65)c32)oc2ccccc24)cc1 ; c1ccc(-c2ccc(N(c3ccc4c5ccc6c7ccccc7sc6c5n(-c5ccccc5)c4c3)c3cccc4c3-c3ccccc3C43c4ccccc4-c4ccccc43)cc2)cc1 ; CC1(C)c2ccccc2-c2ccc(N(c3ccc4c5ccc6c7ccccc7sc6c5n(-c5ccccc5)c4c3)c3cccc4c3-c3ccccc3C43c4ccccc4-c4ccccc43)cc21]

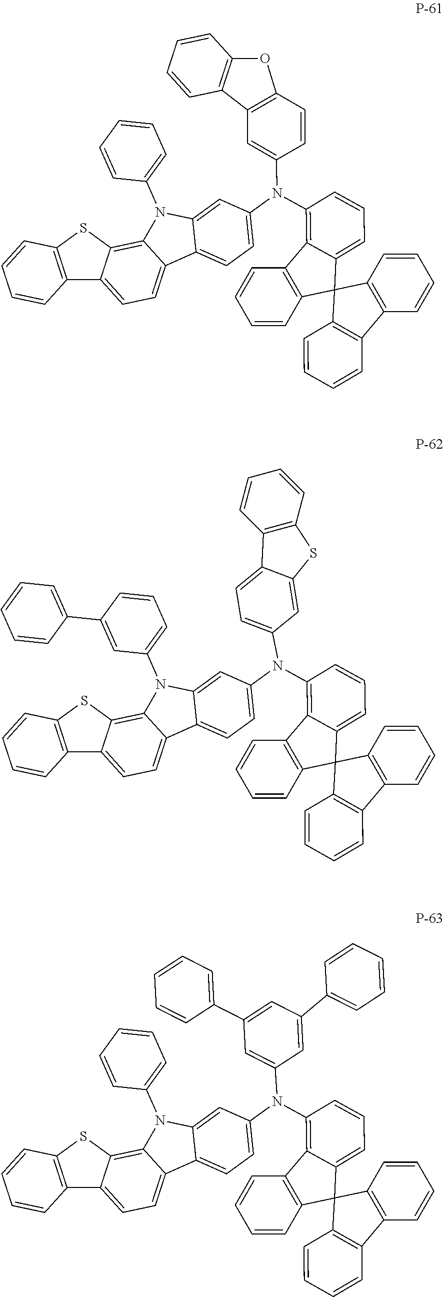 [SMILES: c1ccc(-n2c3cc(N(c4ccc5oc6ccccc6c5c4)c4cccc5c4-c4ccccc4C54c5ccccc5-c5ccccc54)ccc3c3ccc4c5ccccc5sc4c32)cc1 ; c1ccc(-c2cccc(-n3c4cc(N(c5ccc6c(c5)sc5ccccc56)c5cccc6c5-c5ccccc5C65c6ccccc6-c6ccccc65)ccc4c4ccc5c6ccccc6sc5c43)c2)cc1 ; c1ccc(-c2cc(-c3ccccc3)cc(N(c3ccc4c5ccc6c7ccccc7sc6c5n(-c5ccccc5)c4c3)c3cccc4c3-c3ccccc3C43c4ccccc4-c4ccccc43)c2)cc1]

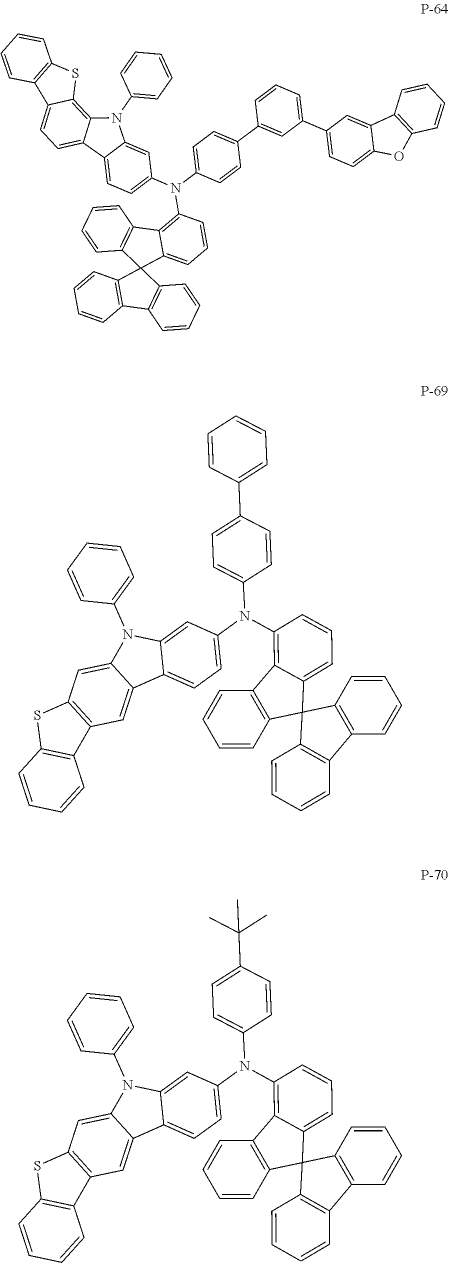 [SMILES: c1ccc(-n2c3cc(N(c4ccc(-c5cccc(-c6ccc7oc8ccccc8c7c6)c5)cc4)c4cccc5c4-c4ccccc4C54c5ccccc5-c5ccccc54)ccc3c3ccc4c5ccccc5sc4c32)cc1 ; c1ccc(-c2ccc(N(c3ccc4c5cc6c(cc5n(-c5ccccc5)c4c3)sc3ccccc36)c3cccc4c3-c3ccccc3C43c4ccccc4-c4ccccc43)cc2)cc1 ; CC(C)(C)c1ccc(N(c2ccc3c4cc5c(cc4n(-c4ccccc4)c3c2)sc2ccccc25)c2cccc3c2-c2ccccc2C32c3ccccc3-c3ccccc32)cc1]

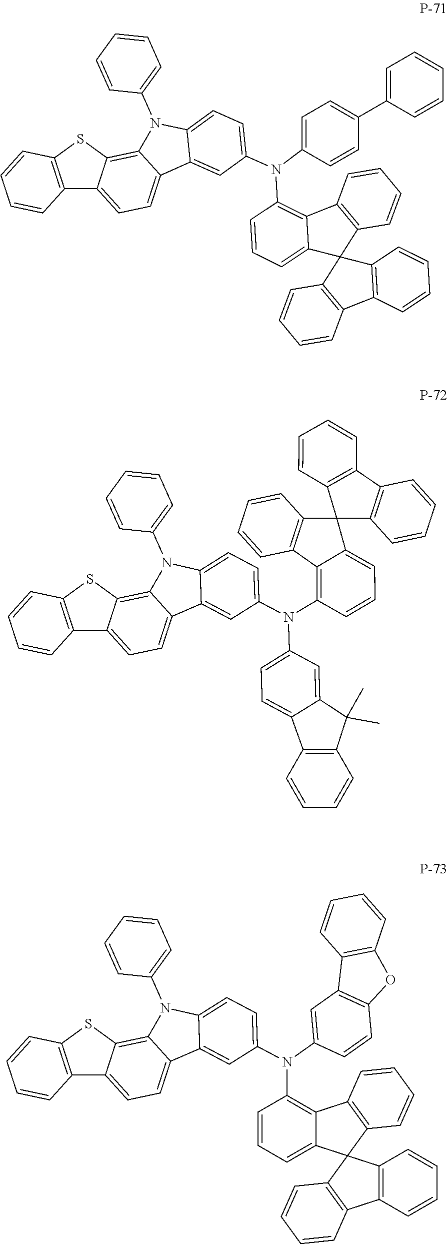 [SMILES: c1ccc(-c2ccc(N(c3ccc4c(c3)c3ccc5c6ccccc6sc5c3n4-c3ccccc3)c3cccc4c3-c3ccccc3C43c4ccccc4-c4ccccc43)cc2)cc1 ; CC1(C)c2ccccc2-c2ccc(N(c3ccc4c(c3)c3ccc5c6ccccc6sc5c3n4-c3ccccc3)c3cccc4c3-c3ccccc3C43c4ccccc4-c4ccccc43)cc21 ; c1ccc(-n2c3ccc(N(c4ccc5oc6ccccc6c5c4)c4cccc5c4-c4ccccc4C54c5ccccc5-c5ccccc54)cc3c3ccc4c5ccccc5sc4c32)cc1]

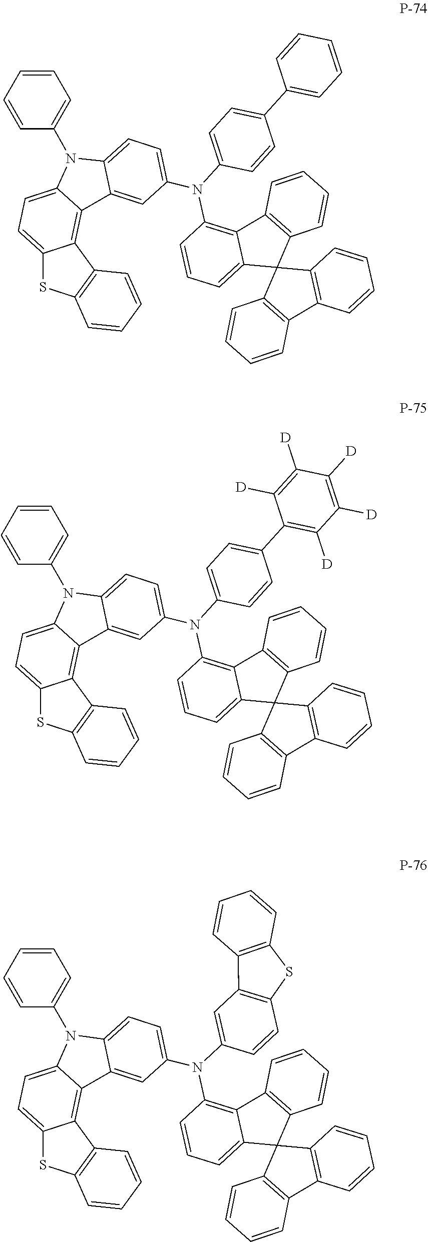 [SMILES: c1ccc(-c2ccc(N(c3ccc4c(c3)c3c5c(ccc3n4-c3ccccc3)sc3ccccc35)c3cccc4c3-c3ccccc3C43c4ccccc4-c4ccccc43)cc2)cc1 ; [2H]c1c([2H])c([2H])c(-c2ccc(N(c3ccc4c(c3)c3c5c(ccc3n4-c3ccccc3)sc3ccccc35)c3cccc4c3-c3ccccc3C43c4ccccc4-c4ccccc43)cc2)c([2H])c1[2H] ; c1ccc(-n2c3ccc(N(c4ccc5sc6ccccc6c5c4)c4cccc5c4-c4ccccc4C54c5ccccc5-c5ccccc54)cc3c3c4c(ccc32)sc2ccccc24)cc1]

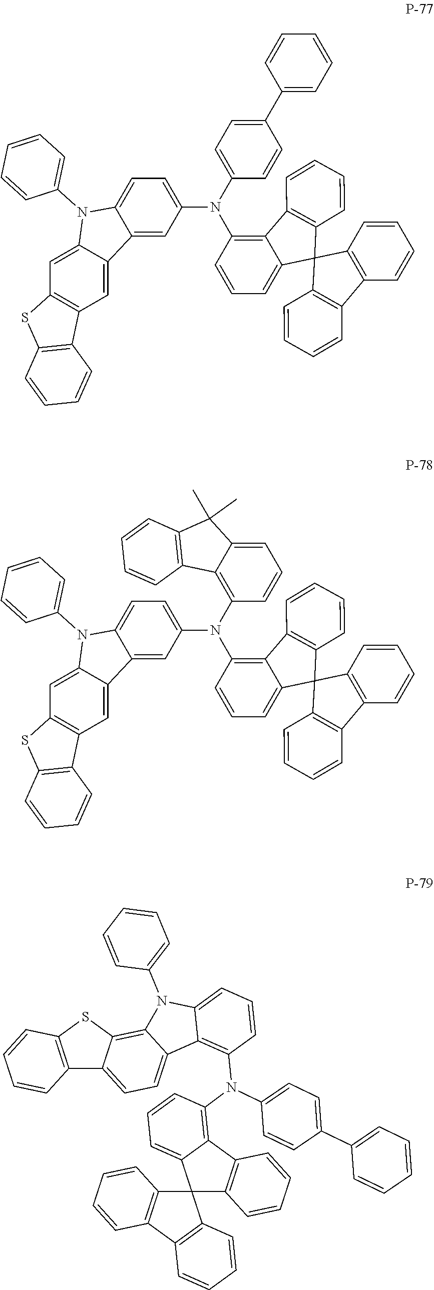 [SMILES: c1ccc(-c2ccc(N(c3ccc4c(c3)c3cc5c(cc3n4-c3ccccc3)sc3ccccc35)c3cccc4c3-c3ccccc3C43c4ccccc4-c4ccccc43)cc2)cc1 ; CC1(C)c2ccccc2-c2c(N(c3ccc4c(c3)c3cc5c(cc3n4-c3ccccc3)sc3ccccc35)c3cccc4c3-c3ccccc3C43c4ccccc4-c4ccccc43)cccc21 ; c1ccc(-c2ccc(N(c3cccc4c3-c3ccccc3C43c4ccccc4-c4ccccc43)c3cccc4c3c3ccc5c6ccccc6sc5c3n4-c3ccccc3)cc2)cc1]

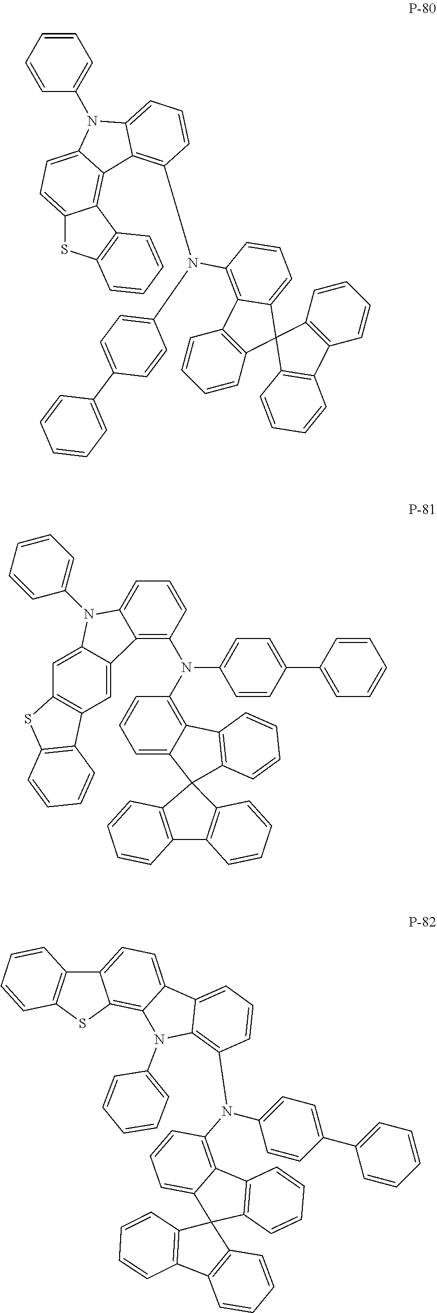 [SMILES: c1ccc(-c2ccc(N(c3cccc4c3-c3ccccc3C43c4ccccc4-c4ccccc43)c3cccc4c3c3c5c(ccc3n4-c3ccccc3)sc3ccccc35)cc2)cc1 ; c1ccc(-c2ccc(N(c3cccc4c3-c3ccccc3C43c4ccccc4-c4ccccc43)c3cccc4c3c3cc5c(cc3n4-c3ccccc3)sc3ccccc35)cc2)cc1 ; c1ccc(-c2ccc(N(c3cccc4c3-c3ccccc3C43c4ccccc4-c4ccccc43)c3cccc4c5ccc6c7ccccc7sc6c5n(-c5ccccc5)c34)cc2)cc1]

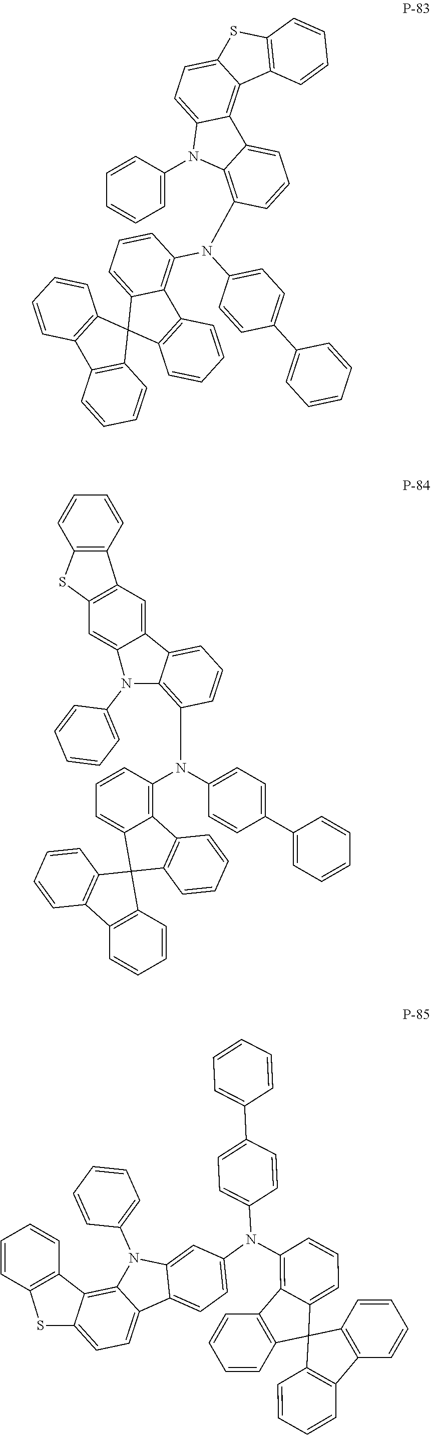 [SMILES: c1ccc(-c2ccc(N(c3cccc4c3-c3ccccc3C43c4ccccc4-c4ccccc43)c3cccc4c5c6c(ccc5n(-c5ccccc5)c34)sc3ccccc36)cc2)cc1 ; c1ccc(-c2ccc(N(c3cccc4c3-c3ccccc3C43c4ccccc4-c4ccccc43)c3cccc4c5cc6c(cc5n(-c5ccccc5)c34)sc3ccccc36)cc2)cc1 ; c1ccc(-c2ccc(N(c3ccc4c5ccc6sc7ccccc7c6c5n(-c5ccccc5)c4c3)c3cccc4c3-c3ccccc3C43c4ccccc4-c4ccccc43)cc2)cc1]

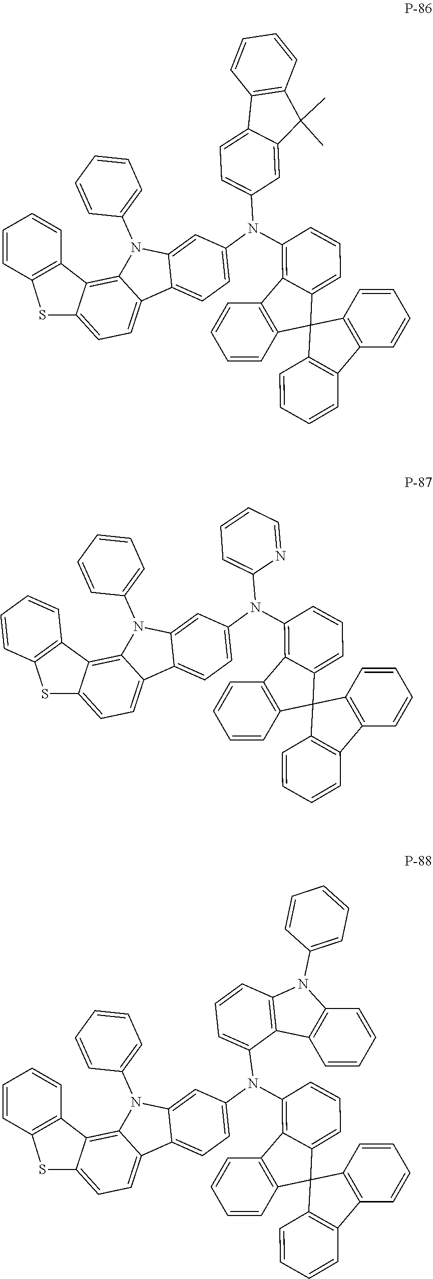 [SMILES: CC1(C)c2ccccc2-c2ccc(N(c3ccc4c5ccc6sc7ccccc7c6c5n(-c5ccccc5)c4c3)c3cccc4c3-c3ccccc3C43c4ccccc4-c4ccccc43)cc21 ; c1ccc(-n2c3cc(N(c4ccccn4)c4cccc5c4-c4ccccc4C54c5ccccc5-c5ccccc54)ccc3c3ccc4sc5ccccc5c4c32)cc1 ; c1ccc(-n2c3ccccc3c3c(N(c4ccc5c6ccc7sc8ccccc8c7c6n(-c6ccccc6)c5c4)c4cccc5c4-c4ccccc4C54c5ccccc5-c5ccccc54)cccc32)cc1]

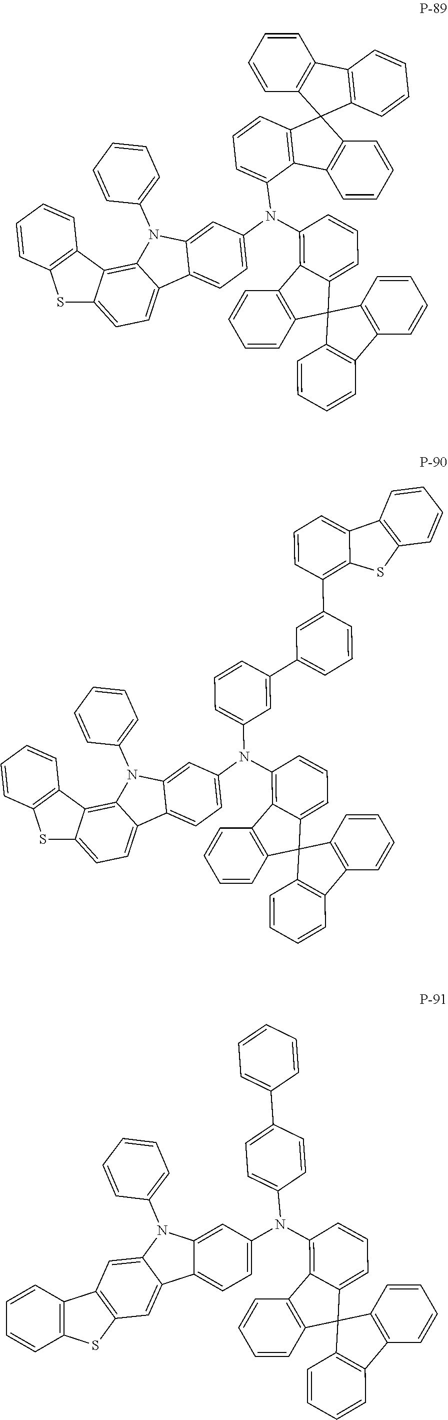 [SMILES: c1ccc(-n2c3cc(N(c4cccc5c4-c4ccccc4C54c5ccccc5-c5ccccc54)c4cccc5c4-c4ccccc4C54c5ccccc5-c5ccccc54)ccc3c3ccc4sc5ccccc5c4c32)cc1 ; c1ccc(-n2c3cc(N(c4cccc(-c5cccc(-c6cccc7c6sc6ccccc67)c5)c4)c4cccc5c4-c4ccccc4C54c5ccccc5-c5ccccc54)ccc3c3ccc4sc5ccccc5c4c32)cc1 ; c1ccc(-c2ccc(N(c3ccc4c5cc6sc7ccccc7c6cc5n(-c5ccccc5)c4c3)c3cccc4c3-c3ccccc3C43c4ccccc4-c4ccccc43)cc2)cc1]

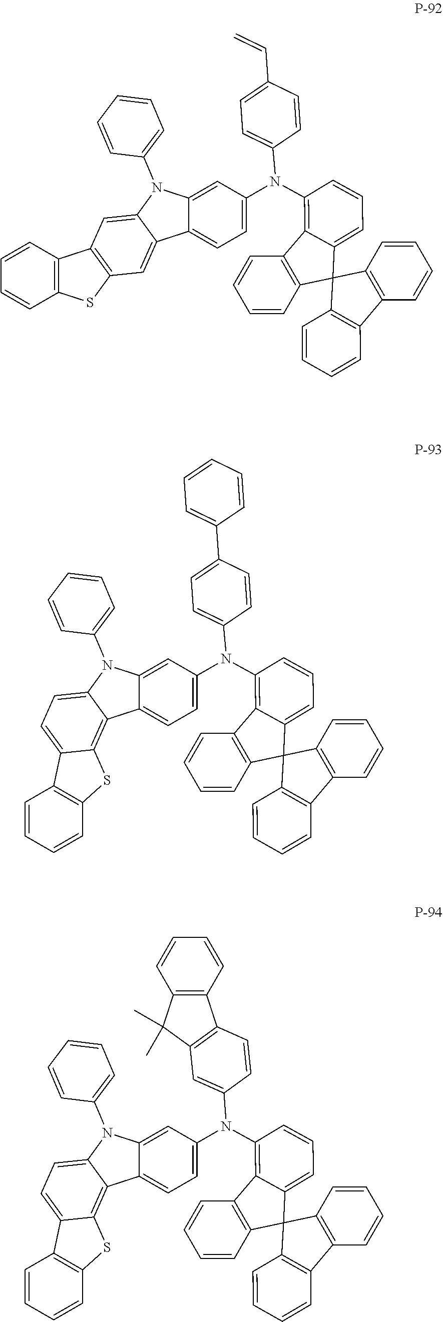 [SMILES: C=Cc1ccc(N(c2ccc3c4cc5sc6ccccc6c5cc4n(-c4ccccc4)c3c2)c2cccc3c2-c2ccccc2C32c3ccccc3-c3ccccc32)cc1 ; c1ccc(-c2ccc(N(c3ccc4c5c6sc7ccccc7c6ccc5n(-c5ccccc5)c4c3)c3cccc4c3-c3ccccc3C43c4ccccc4-c4ccccc43)cc2)cc1 ; CC1(C)c2ccccc2-c2ccc(N(c3ccc4c5c6sc7ccccc7c6ccc5n(-c5ccccc5)c4c3)c3cccc4c3-c3ccccc3C43c4ccccc4-c4ccccc43)cc21]

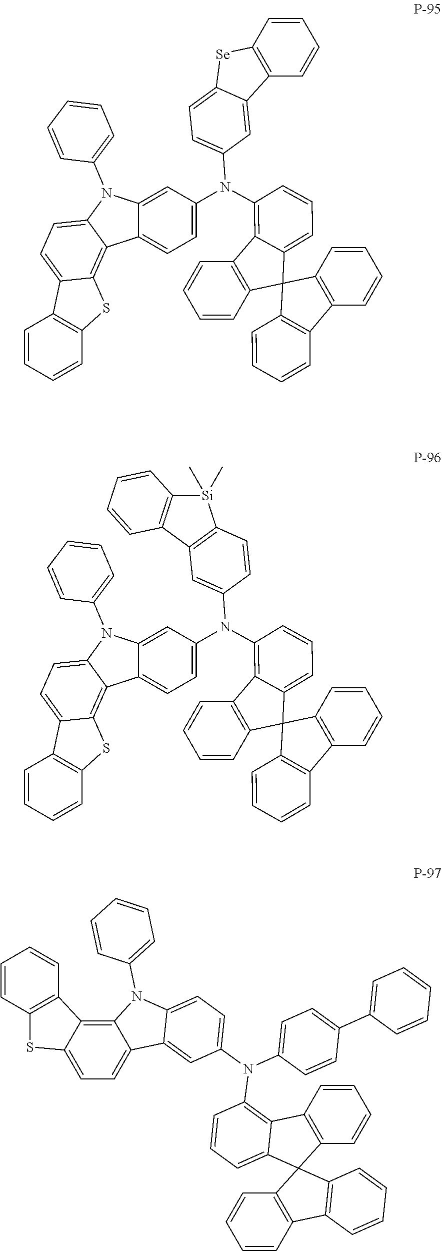 [SMILES: c1ccc(-n2c3cc(N(c4ccc5[se]c6ccccc6c5c4)c4cccc5c4-c4ccccc4C54c5ccccc5-c5ccccc54)ccc3c3c4sc5ccccc5c4ccc32)cc1 ; C[Si]1(C)c2ccccc2-c2cc(N(c3ccc4c5c6sc7ccccc7c6ccc5n(-c5ccccc5)c4c3)c3cccc4c3-c3ccccc3C43c4ccccc4-c4ccccc43)ccc21 ; c1ccc(-c2ccc(N(c3ccc4c(c3)c3ccc5sc6ccccc6c5c3n4-c3ccccc3)c3cccc4c3-c3ccccc3C43c4ccccc4-c4ccccc43)cc2)cc1]

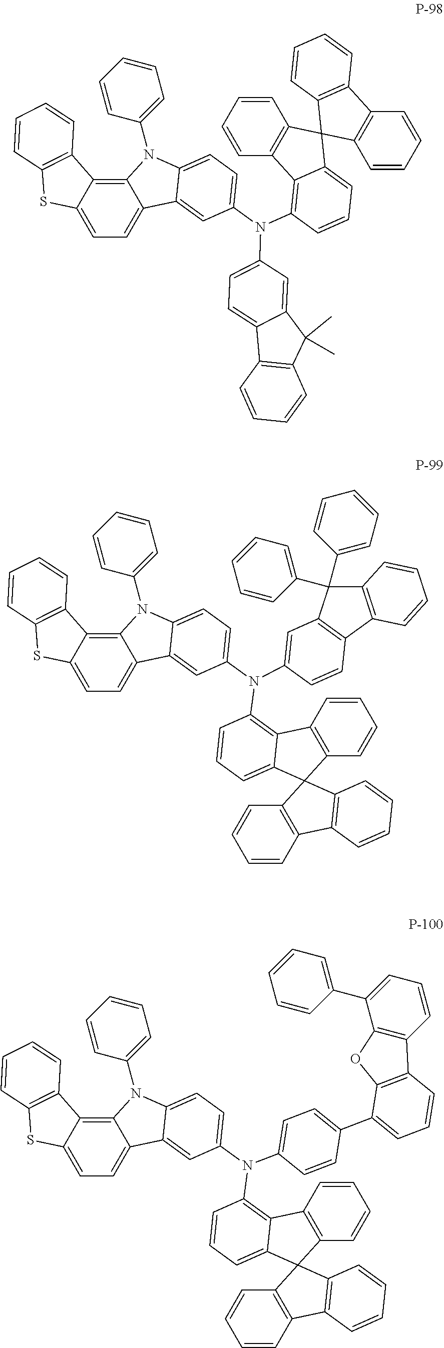 [SMILES: CC1(C)c2ccccc2-c2ccc(N(c3ccc4c(c3)c3ccc5sc6ccccc6c5c3n4-c3ccccc3)c3cccc4c3-c3ccccc3C43c4ccccc4-c4ccccc43)cc21 ; c1ccc(-n2c3ccc(N(c4ccc5c(c4)C(c4ccccc4)(c4ccccc4)c4ccccc4-5)c4cccc5c4-c4ccccc4C54c5ccccc5-c5ccccc54)cc3c3ccc4sc5ccccc5c4c32)cc1 ; c1ccc(-c2cccc3c2oc2c(-c4ccc(N(c5ccc6c(c5)c5ccc7sc8ccccc8c7c5n6-c5ccccc5)c5cccc6c5-c5ccccc5C65c6ccccc6-c6ccccc65)cc4)cccc23)cc1]

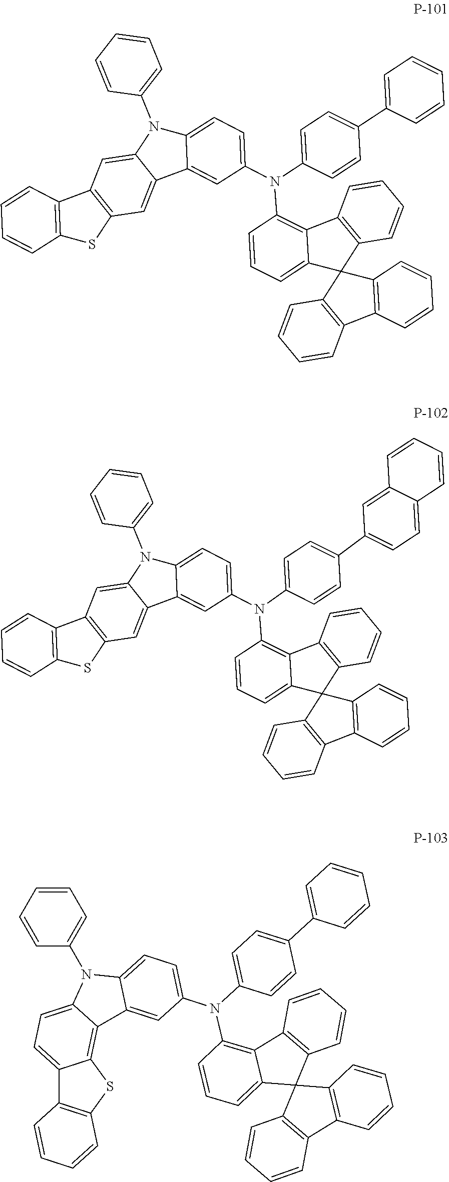 [SMILES: c1ccc(-c2ccc(N(c3ccc4c(c3)c3cc5sc6ccccc6c5cc3n4-c3ccccc3)c3cccc4c3-c3ccccc3C43c4ccccc4-c4ccccc43)cc2)cc1 ; c1ccc(-n2c3ccc(N(c4ccc(-c5ccc6ccccc6c5)cc4)c4cccc5c4-c4ccccc4C54c5ccccc5-c5ccccc54)cc3c3cc4sc5ccccc5c4cc32)cc1 ; c1ccc(-c2ccc(N(c3ccc4c(c3)c3c5sc6ccccc6c5ccc3n4-c3ccccc3)c3cccc4c3-c3ccccc3C43c4ccccc4-c4ccccc43)cc2)cc1]

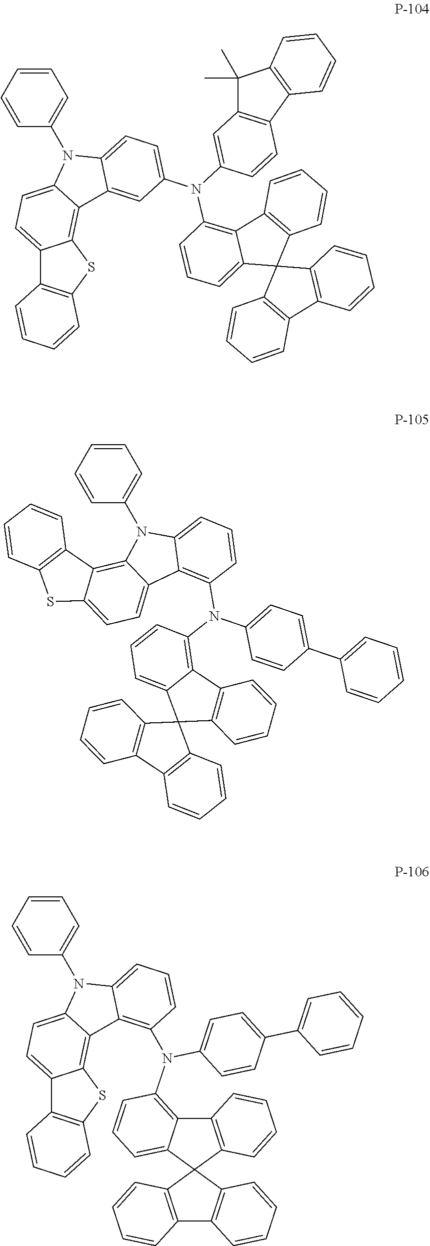 [SMILES: CC1(C)c2ccccc2-c2ccc(N(c3ccc4c(c3)c3c5sc6ccccc6c5ccc3n4-c3ccccc3)c3cccc4c3-c3ccccc3C43c4ccccc4-c4ccccc43)cc21 ; c1ccc(-c2ccc(N(c3cccc4c3-c3ccccc3C43c4ccccc4-c4ccccc43)c3cccc4c3c3ccc5sc6ccccc6c5c3n4-c3ccccc3)cc2)cc1 ; c1ccc(-c2ccc(N(c3cccc4c3-c3ccccc3C43c4ccccc4-c4ccccc43)c3cccc4c3c3c5sc6ccccc6c5ccc3n4-c3ccccc3)cc2)cc1]

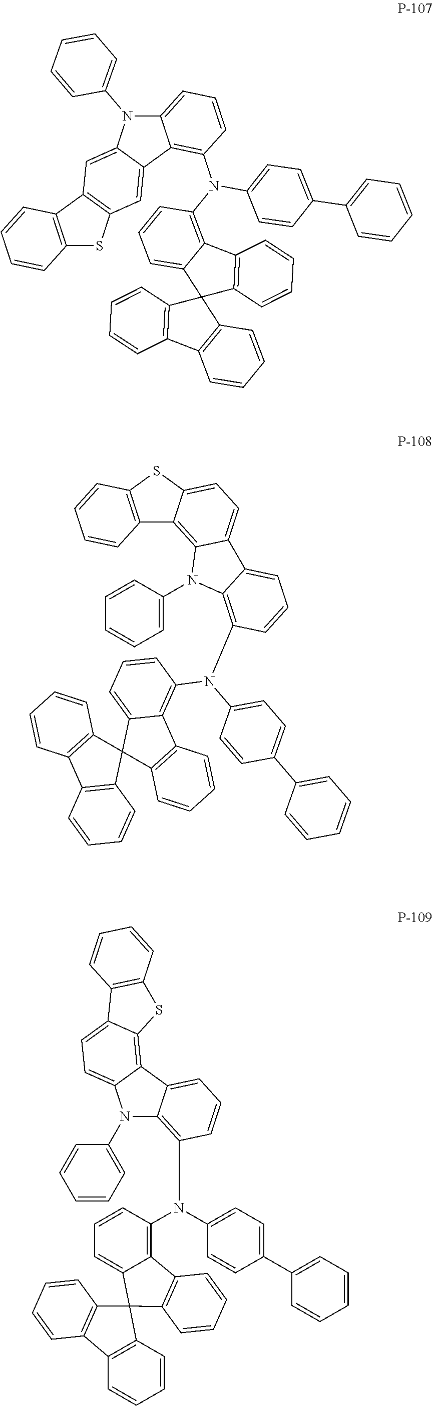 [SMILES: c1ccc(-c2ccc(N(c3cccc4c3-c3ccccc3C43c4ccccc4-c4ccccc43)c3cccc4c3c3cc5sc6ccccc6c5cc3n4-c3ccccc3)cc2)cc1 ; c1ccc(-c2ccc(N(c3cccc4c3-c3ccccc3C43c4ccccc4-c4ccccc43)c3cccc4c5ccc6sc7ccccc7c6c5n(-c5ccccc5)c34)cc2)cc1 ; c1ccc(-c2ccc(N(c3cccc4c3-c3ccccc3C43c4ccccc4-c4ccccc43)c3cccc4c5c6sc7ccccc7c6ccc5n(-c5ccccc5)c34)cc2)cc1]

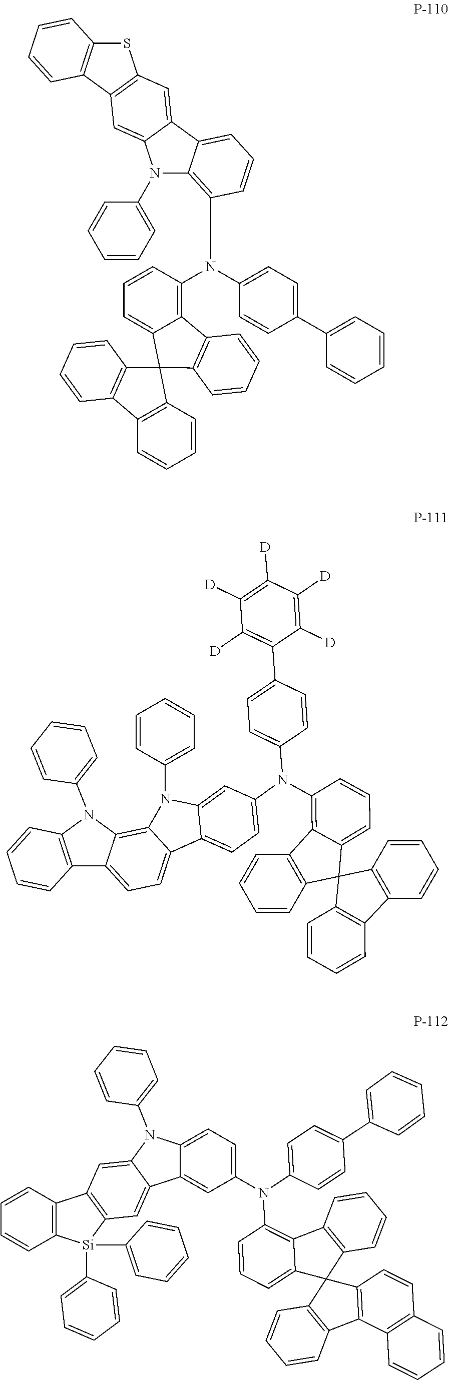 [SMILES: c1ccc(-c2ccc(N(c3cccc4c3-c3ccccc3C43c4ccccc4-c4ccccc43)c3cccc4c5cc6sc7ccccc7c6cc5n(-c5ccccc5)c34)cc2)cc1 ; [2H]c1c([2H])c([2H])c(-c2ccc(N(c3ccc4c5ccc6c7ccccc7n(-c7ccccc7)c6c5n(-c5ccccc5)c4c3)c3cccc4c3-c3ccccc3C43c4ccccc4-c4ccccc43)cc2)c([2H])c1[2H] ; c1ccc(-c2ccc(N(c3ccc4c(c3)c3cc5c(cc3n4-c3ccccc3)-c3ccccc3[Si]5(c3ccccc3)c3ccccc3)c3cccc4c3-c3ccccc3C43c4ccccc4-c4c3ccc3ccccc43)cc2)cc1]

In another embodiment, the present disclosure provides a compound for an organic electric element, the compound being represented by Formula 1 above.

In still another embodiment, the present disclosure provides an organic electric element containing the compound represented by Formula 1.

Here, the organic electric element may include: a first electrode; a second electrode; and an organic material layer positioned between the first electrode and the second electrode, wherein the organic material layer may contain a compound represented by Formula 1, and the compound represented by Formula 1 may be contained in at least one layer of a hole injection layer, a hole transport layer, a light emitting auxiliary layer, a light emitting layer, an electron transport auxiliary layer, an electron auxiliary layer, an electron transport layer, and an electron injection layer of the organic material layer. Especially, the compound represented by Formula 1 may be contained in the hole transport layer or the light emitting auxiliary layer.

That is, the compound represented by Formula 1 may be used as a material for a hole injection layer, a hole transport layer, a light emitting auxiliary layer, a light emitting layer, an electron transport auxiliary layer, an electron auxiliary layer, an electron transport layer, or an electron injection layer of the organic material layer. Especially, the compound represented by Formula 1 may be used as a material for the hole transport layer or the light emitting auxiliary layer. The present disclosure provides, specifically, an organic electric element including one of the compounds represented by Formula 1 in the organic material layer, and more specifically, organic electric elements including one of the compounds represented by the above individual Formulas (P-1 to P-112) in the organic material layer.

In still another embodiment, the present disclosure provides an organic electric element characterized in that the compound is contained alone, two or more different kinds of the compounds are contained as a combination, or the compound is contained together with other compounds as a combination of two or more in at least one layer of the hole injection layer, the hole transport layer, the light emitting auxiliary layer, the light emitting layer, the electron transport auxiliary layer, the electron auxiliary layer, the electron transport layer, and the electron injection layer of the organic material layer. In other words, the compound corresponding to Formula 1 may be contained alone, a mixture of two or more kinds of the compounds of Formula 1 may be contained, or a mixture of the compound of claims 1 to 3 and a compound not corresponding to the present disclosure may be contained in each of the layers. Here, the compound not correspond to the present disclosure may be a single compound or two or more kinds of compounds. Here, when the compound is contained together with other compounds as a combination of two or more kinds of compounds, the other compounds may be compounds that are already known for each organic material layer, or compounds to be developed in the future. Here, the compounds contained in the organic material layer may be composed of only the same kind of compounds, or a mixture of two or more kinds of different compounds represented by Formula 1. More preferably, the organic material layer includes a light emitting layer and a light emitting auxiliary layer, and the light emitting layer contains a phosphorescent green emitter. The compound is contained in the light emitting auxiliary layer.

In still another embodiment of the present disclosure, the present disclosure provides an organic electric element further including a light efficiency improvement layer, which is formed on at least one between one surface of the first electrode, which is the opposite side to the organic material layer, and one surface of the second electrode, which is the opposite side to the organic material layer.

Hereinafter, synthesis examples of the compound represented by Formula 1 and manufacturing examples of the organic electric element according to the present disclosure will be described in detail by way of examples. However, the present disclosure is not limited to the following examples.

### SYNTHESIS EXAMPLES

Compounds (final products) represented by Formula 1 according to the present disclosure are synthesized by a reaction of Sub 1 and Sub 2 as shown in Reaction Scheme 1 below, but are not limited thereto.

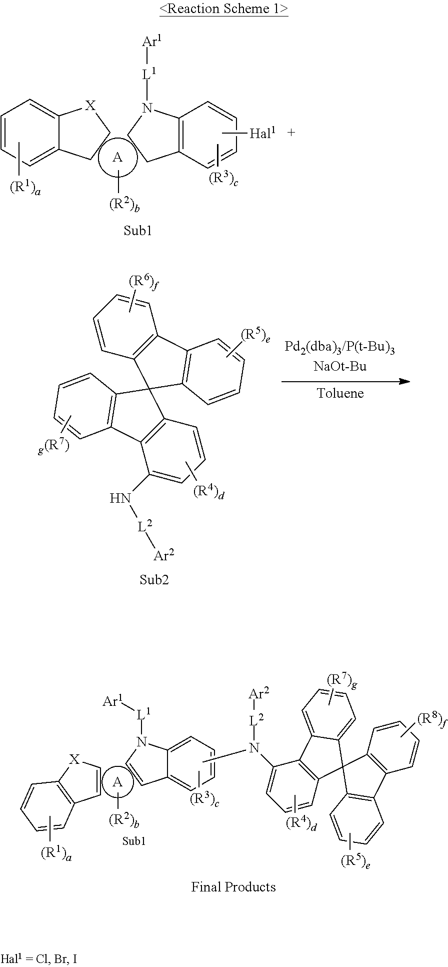

I. Synthesis of Sub 1

Sub 1 of Reaction Scheme 1 above may be synthesized by the reaction pathway of Reaction Scheme 2, but is not limited thereto.

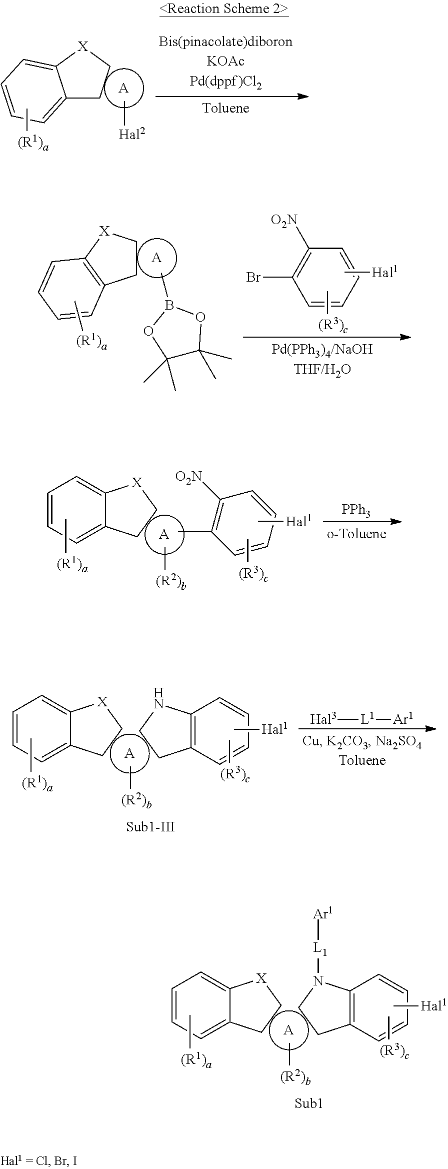

Synthesis examples of specific compounds pertaining to Sub 1 are as follows.

### 1. Synthesis Example of Sub 1-1

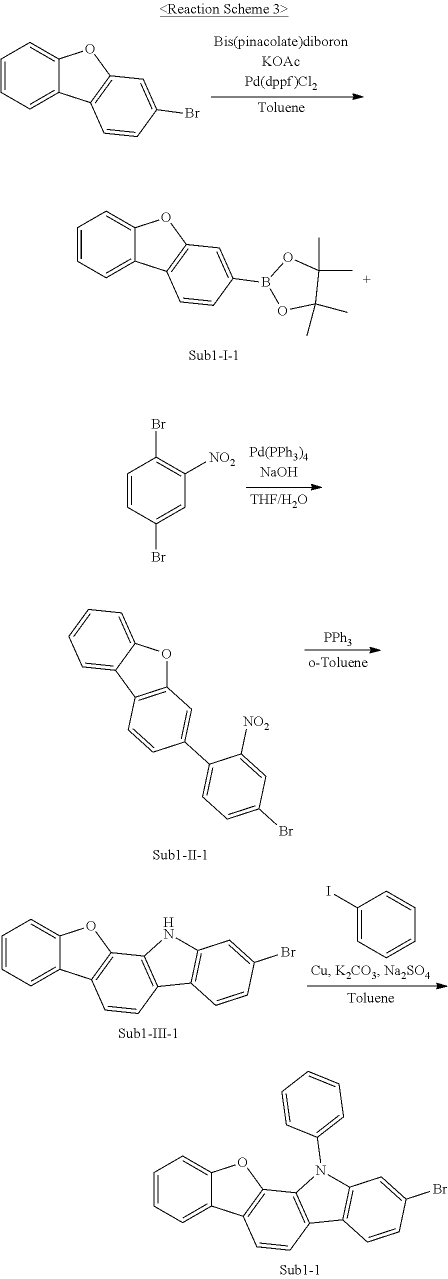

(1) Synthesis of Sub 1-I-1

3-Bromodibenzo[b,d]furan (100.00 g, 404.71 mmol), bis(pinacolato)diboron (113.05 g, 445.2 mmol), KOAc (119.15 g, 445.2 mmol), and PdCl₂ (ddpf) (9.92 g, 12.1 mmol) were dissolved in Toluene (2024 mL), and then refluxed at 120° C. for 12 hours. Upon the completion of the reaction, the reaction product was cooled to normal temperature, extracted with CH₂Cl₂, and washed with water. The organic layer was dried over MgSO₄ and concentrated, and then the formed organic material was recrystallized using CH₂Cl₂ and methanol solvents to give a desired product (119.05 g, 80%).

(2) Synthesis of Sub 1-II-1

After Sub 1-I-1 (28.00 g, 95.2 mmol) obtained from the synthesis was dissolved in THF (900 ml) in a round-bottom flask, 1,4-dibromo-2-nitrobenzene (40.11 g, 142.8 mmol), Pd(PPh₃)₄ (5.50 g, 4.8 mmol), K₂CO₃ (49.47 g, 258.6 mmol), and water (300 ml) were added, followed by stirring at 80° C. Upon completion of the reaction, the reaction product was extracted with CH₂Cl₂ and water. The organic layer was dried over MgSO₄ and concentrated, and then the thus formed compound was subjected to silica gel column and recrystallization to give a product 27.34 g (yield: 78%).

(3) Synthesis of Sub 1-III-1

After Sub 1-II-1 (22.00 g, 59.8 mmol) obtained from the synthesis was dissolved in o-dichlorobenzene (299 ml) in a round-bottom flask, triphenylphosphine (39.18 g, 149.4 mmol) was added, followed by stirring at 200° C. Upon completion of the reaction, o-dichlorobenzene was removed through distillation, followed by extraction with CH₂Cl₂ and water. The organic layer was dried over MgSO₄ and concentrated, and then the formed compound was subjected to silica gel column and recrystallization to give a product 13.66 g (yield: 68%).

(4) Synthesis of Sub 1-1

After Sub 1-III-1 (13.66 g, 40.6 mmol) obtained from the synthesis was dissolved in nitrobenzene (508 ml) in a round-bottom flask, iodobenzene (12.43 g, 60.9 mmol), Na₂SO₄ (5.77 g, 40.6 mmol), K₂CO₃ (5.62 g, 40.6 mmol), and Cu (0.77 g, 12.2 mmol) were added, followed by stirring at 200° C. Upon completion of the reaction, nitrobenzene was removed through distillation, followed by extraction with CH₂Cl₂ and water. The organic layer was dried over MgSO₄ and concentrated, and then the formed compound was subjected to silica gel column and recrystallization to give a product 12.87 g (yield: 75%).

### 2. Synthesis Example of Sub 1-4

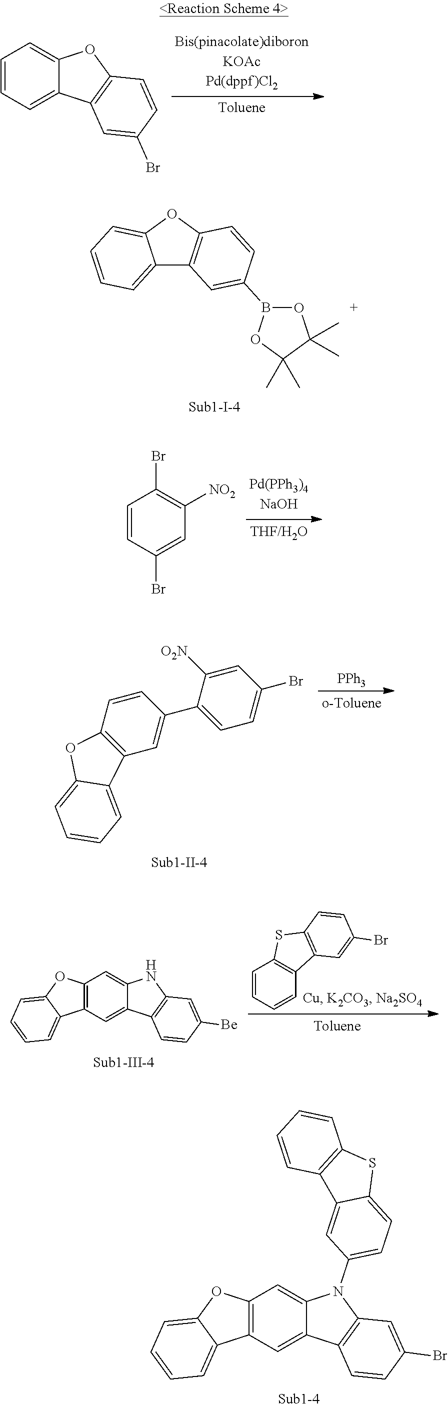

(1) Synthesis of Sub 1-I-4

2-bromodibenzo[b,d]furan (34.00 g, 137.6 mmol), bis(pinacolato)diboron (38.44 g, 151.4 mmol), KOAc (40.51 g, 412.8 mmol)(6.52, PdCl₂ (dppf) (3.37 g, 4.1 mmol), and Toluene (688 ml) were subjected to the synthesis method of Sub 1-I-1 to give a product (33.19 g, 82%).

(2) Synthesis of Sub 1-II-4

After Sub 1-I-4 (33.19 g, 112.8 mmol) obtained from the synthesis was dissolved in THF (900 ml) in a round-bottom flask, 1,4-dibromo-2-nitrobenzene (47.54 g, 169.2 mmol), Pd(PPh₃)₄ (6.52 g, 5.6 mmol), K₂CO₃ (46.78 g, 338.5 mmol), and water (300 ml) were added, and subjected to the synthesis method of Sub 1-II-1 to give a product 30.30 g (yield: 710).

(3) Synthesis of Sub 1-III-4

After Sub 1-II-4 (30.30 g, 82.3 mmol) obtained from the synthesis was dissolved in o-dichlorobenzene (299 ml) in a round-bottom flask, triphenylphosphine (53.96 g, 205.7 mmol) was added, and subjected to the synthesis method of Sub 1-III-1 to give a product 11.62 g (yield: 42%).

(4) Synthesis of Sub 1-4

After the Sub 1-III-4 (11.62 g, 34.6 mmol) obtained from the synthesis was dissolved in nitrobenzene (432 ml) in a round-bottom flask, 2-bromodibenzo[b,d]thiophene (13.64 g, 51.8 mmol), Na₂SO₄ (4.91 g, 34.6 mmol), K₂CO₃ (4.78 g, 34.6 mmol), and Cu (0.66 g, 10.4 mmol) were added, and subjected to the synthesis method of Sub 1-1 to give a product 9.68 g (yield: 54%).

### 3. Synthesis Example of Sub 1-7

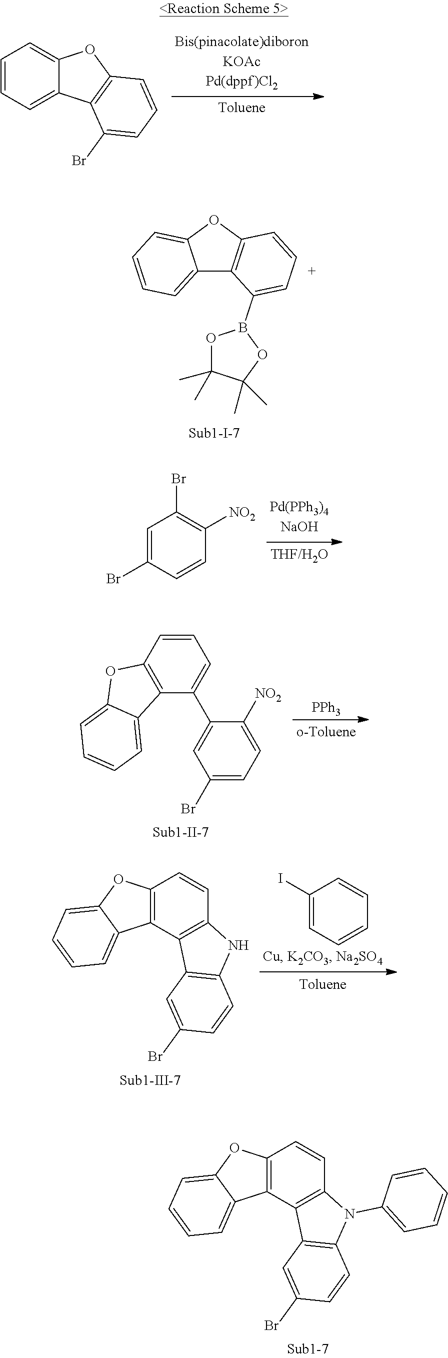

(1) Synthesis of Sub 1-I-7

1-Bromodibenzo[b,d]furan (80.0 g, 323.8 mmol), bis(pinacolato)diboron (90.44 g, 356.1 mmol), KOAc (95.32 g, 971.3 mmol), PdCl₂(dppf) (7.93 g, 9.7 mmol) and Toluene (1600 mL) were subjected to the synthesis method of Sub 1-I-1 to give a product 71.43 g (yield: 75%).

(2) Synthesis of Sub 1-II-7

After Sub 1-I-7 obtained from the synthesis was dissolved in THF (1200 ml) in a round-bottom flask, 1,5-dibromo-2-nitrobenzene (51.15 g, 182.1 mmol), Pd(PPh₃)₄ (7.01 g, 6.1 mmol), K₂CO₃ (50.33 g, 364.2 mmol), and water (300 ml) were added, and subjected to the synthesis method of Sub 1-II-1 to give a product 32.14 g (yield: 70%).

(3) Synthesis of Sub 1-III-7

After Sub 1-II-7 (32.14 g, 87.3 mmol) obtained from the synthesis was dissolved in o-dichlorobenzene (400 ml) in a round-bottom flask, triphenylphosphine (57.24 g, 218.2 mmol) was added, and subjected to the synthesis method of Sub 1-III-1 to give a product 19.37 g (yield: 66%).

(4) Synthesis of Sub 1-7

After Sub 1-III-7 (19.37 g, 57.6 mmol) obtained from the synthesis was dissolved in nitrobenzene (720 ml) in a round-bottom flask, iodobenzene (17.63 g, 86.4 mmol), Na₂SO₄ (8.18 g, 57.6 mmol), K₂CO₃ (7.96 g, 57.6 mmol), and Cu (1.10 g, 17.3 mmol) were added, and subjected to the synthesis method of Sub 1-1 to give a product 17.52 g (yield: 72%).

### 4. Synthesis Example of Sub 1-9

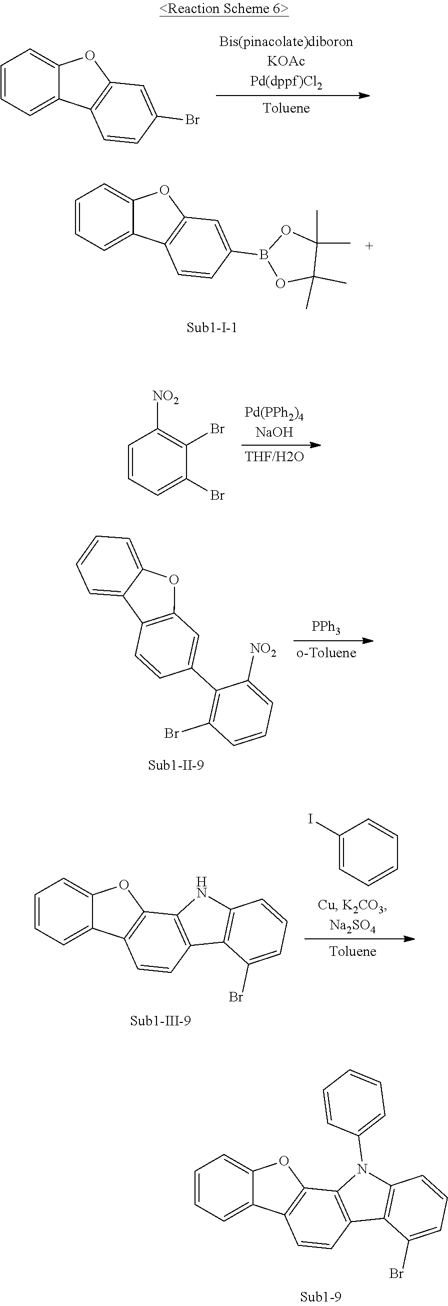

(1) Synthesis of Sub 1-II-9

After Sub 1-I-1 (25.60 g, 87.0 mmol) obtained from the synthesis was dissolved in THF (800 ml) in a round-bottom flask, 1,2-dibromo-3-nitrobenzene (36.67 g, 130.5 mmol), Pd(PPh₃)₄ (5.03 g, 4.4 mmol), K₂CO₃ (36.08 g, 261.1 mmol), and water (400 ml) were added, and subjected to the synthesis method of Sub 1-II-1 to give a product 25.31 g (yield: 79%).

(2) Synthesis of Sub 1-III-9

After Sub 1-II-9 (25.31 g, 68.7 mmol) obtained from the synthesis was dissolved in o-dichlorobenzene (344 ml) in a round-bottom flask, triphenylphosphine (45.08 g, 171.9 mmol) was added, and subjected to the synthesis method of Sub 1-III-1 to give a product 15.02 g (yield: 65%).

(3) Synthesis of Sub 1-9

After Sub 1-III-9 (15.02 g, 44.7 mmol) obtained from the synthesis was dissolved in nitrobenzene (558 ml) in a round-bottom flask, iodobenzene (13.67 g, 67.0 mmol), Na₂SO₄ (6.35 g, 44.7 mmol), K₂CO₃ (6.17 g, 44.7 mmol), and Cu (0.85 g, 13.4 mmol) were added, and subjected to the synthesis method of Sub 1-1 to give a product 12.45 g (yield: 66%).

### 5. Synthesis Example of Sub 1-13

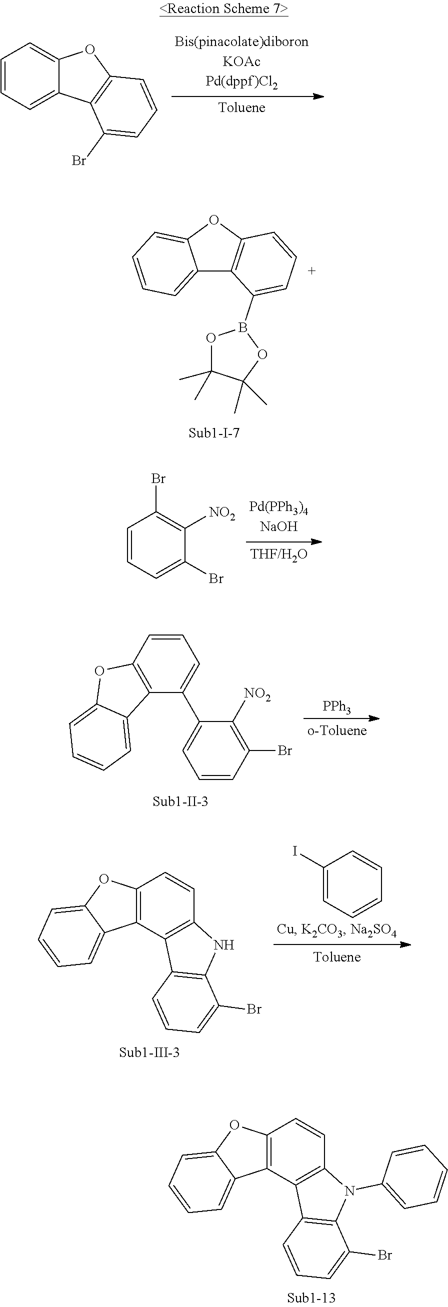

(1) Synthesis of Sub 1-II-13

After Sub 1-I-7 (35.00 g, 141.6 mmol) obtained from the synthesis was dissolved in THF (1200 ml) in a round-bottom flask, 1,3-dibromo-2-nitrobenzene (50.13 g, 178.5 mmol), Pd(PPh₃)₄ (6.87 g, 5.9 mmol), K₂CO₃ (49.33 g, 356.9 mmol)), and water (300 ml) were added, and subjected to the synthesis method of Sub 1-II-1 to give a product 21.60 g (yield: 48%).

(2) Synthesis of Sub 1-III-13

After Sub 1-II-13 (21.00 g, 57.0 mmol) obtained from the synthesis was dissolved in o-dichlorobenzene (285 ml) in a round-bottom flask, triphenylphosphine (37.40 g, 142.6 mmol) was added, and subjected to the synthesis method of Sub 1-III-1 to give a product 13.81 g (yield: 72%).

(3) Synthesis of Sub 1-13

After Sub 1-III-13 (13.81 g, 41.1 mmol) obtained from the synthesis was dissolved in nitrobenzene (513 ml) in a round-bottom flask, iodobenzene (12.57 g, 61.6 mmol), Na₂SO₄ (5.83 g, 41.1 mmol), K₂CO₃ (5.68 g, 41.1 mmol), and Cu (0.78 g, 12.3 mmol) were added, and subjected to the synthesis method of Sub 1-1 to give a product 11.45 g (yield: 66%).

### 6. Synthesis Example of Sub 1-18

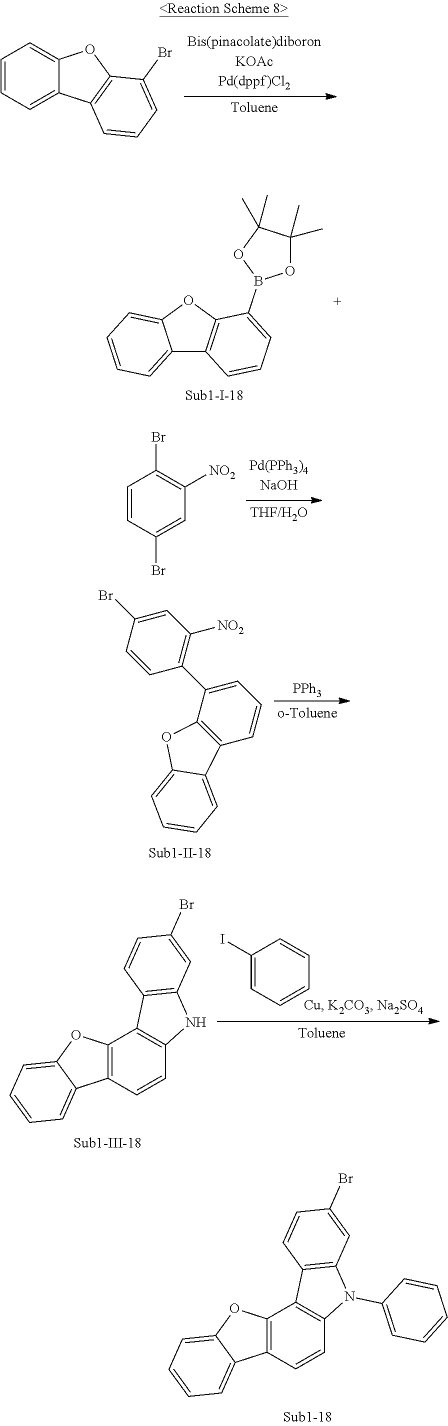

(1) Synthesis of Sub 1-I-18

4-Bromodibenzo[b,d]furan (30 g, 121.4 mmol), bis(pinacolato)diboron (33.91 g, 133.6 mmol), KOAc (35.75 g, 364.2 mmol), PdCl₂(dppf) (2.97 g, 3.6 mmol) and Toluene (600 mL) were added, and subjected to the synthesis method of Sub 1-I-1 to give a product 30.0 g (yield: 84%).

(2) Synthesis of Sub 1-II-18

After Sub 1-I-18 (30.0 g, 102.0 mmol) obtained from the synthesis was dissolved in THF (900 ml) in a round-bottom flask, 1,4-dibromo-2-nitrobenzene (42.97 g, 153.0 mmol), Pd(PPh₃)₄ (5.89 g, 5.1 mmol), K₂CO₃ (42.29 g, 306.0 mmol), and water (300 ml) were added, and subjected to the synthesis method of Sub 1-II-1 to give a product 30.09 g (yield: 78%).

(3) Synthesis of Sub 1-III-18

After Sub 1-II-18 (30.09 g, 81.7 mmol) obtained from the synthesis was dissolved in o-dichlorobenzene (400 ml) in a round-bottom flask, triphenylphosphine (53.59 g, 204.3 mmol) was added, and subjected to the synthesis method of Sub 1-III-1 to give a product 20.61 g (yield: 75%).

(4) Synthesis of Sub 1-18

After Sub 1-III-18 (20.61 g, 61.3 mmol) obtained from the synthesis was dissolved in nitrobenzene (766 ml) in a round-bottom flask, iodobenzene (18.76 g, 61.3 mmol), Na₂SO₄ (8.71 g, 61.3 mmol), K₂CO₃ (8.47 g, 61.3 mmol), and Cu (1.17 g, 18.4 mmol) were added, and subjected to the synthesis method of Sub 1-1 to give a product 19.16 g (yield: 74%).

### 7. Synthesis Example of Sub 1-26

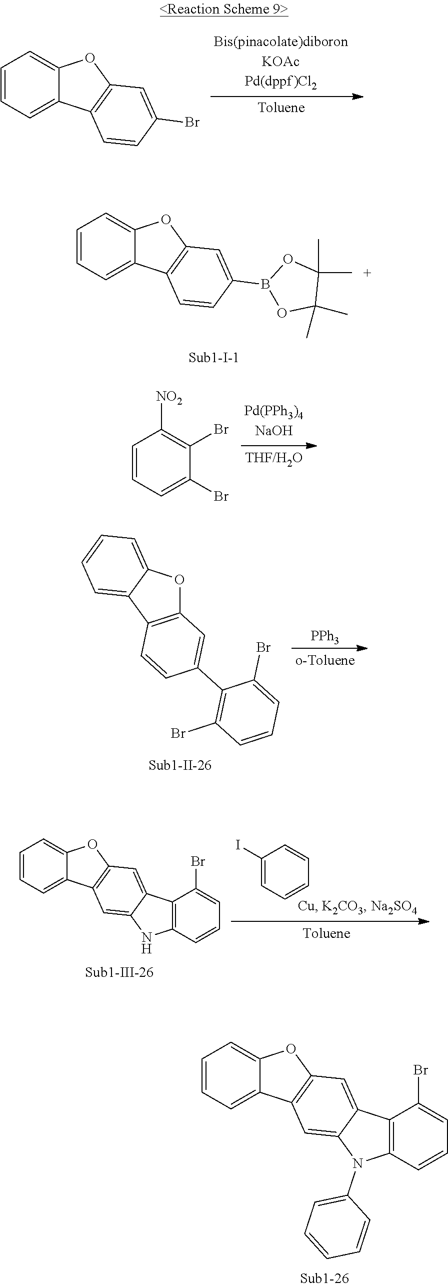

(1) Synthesis of Sub 1-II-26

After Sub 1-I-4 (40.96 g, 161.9 mmol) obtained from the synthesis was dissolved in THF (1200 ml) in a round-bottom flask, 1,2-dibromo-6-nitrobenzene (58.67 g, 208.9 mmol), Pd(PPh₃)₄ (8.05 g, 7.0 mmol), K₂CO₃ (57.73 g, 417.7 mmol), and water (300 ml) were added, and subjected to the synthesis method of Sub 1-II-1 to give a product 40.55 g (yield: 77%).

(2) Synthesis of Sub 1-III-26

After Sub 1-II-26 (40.55 g, 110.1 mmol) obtained from the synthesis was dissolved in o-dichlorobenzene (551 ml) in a round-bottom flask, triphenylphosphine (72.22 g, 275.3 mmol) was added, and subjected to the synthesis method of Sub 1-III-1 to give a product 11.11 g (yield: 30%).

(3) Synthesis of Sub 1-26

After Sub 1-III-26 (11.11 g, mmol) obtained from the synthesis was dissolved in nitrobenzene (413 ml) in a round-bottom flask, iodobenzene (10.11 g, 49.6 mmol), Na₂SO₄ (4.69 g, 33.0 mmol), K₂CO₃ (4.57 g, 33.0 mmol), and Cu (0.63 g, 9.9 mmol) were added, and subjected to the synthesis method of Sub 1-1 to give a product 10.05 g (yield: 72%).

### 8. Synthesis Example of Sub 1-30

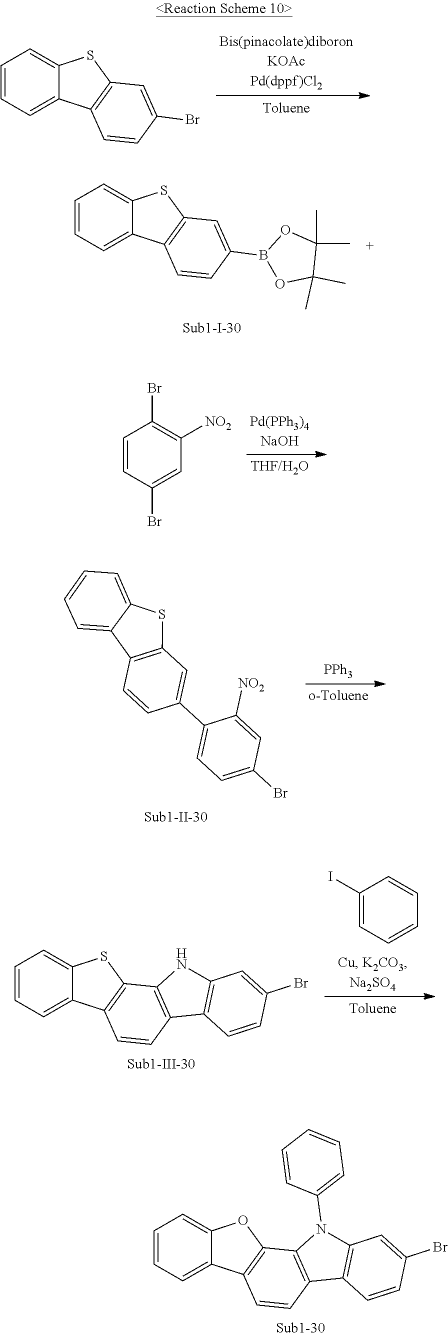

(1) Synthesis of Sub 1-I-30

3-Bromodibenzo[b,d]thiophene (45 g, 171.0 mmol), bis(pinacolato)diboron (47.77 g, 188.1 mmol), KOAc (50.35 g, 513.0 mmol), PdCl₂(dppf) (4.19 g, 5.1 mmol) and Toluene (855 mL) were added, and subjected to the synthesis method of Sub 1-I-1 to give a product 44.56 g (yield: 84%).

(2) Synthesis of Sub 1-II-30

After Sub 1-I-30 (24.76 g, 79.8 mmol) obtained from the synthesis was dissolved in THF (800 ml) in a round-bottom flask 0-∥, 1,4-dibromo-2-nitrobenzene (33.63 g, 119.7 mmol), Pd(PPh₃)₄ (4.61 g, 4.0 mmol), K₂CO₃ (33.09 g, 239.4 mmol), and water (200 ml) were added, and subjected to the synthesis method of Sub 1-II-1 to give a product 21.77 g (yield: 71%).

(3) Synthesis of Sub 1-III-30

After Sub 1-II-30 (21.77 g, 56.7 mmol) obtained from the synthesis was dissolved in o-dichlorobenzene (283 ml) in a round-bottom flask, triphenylphosphine (37.15 g, 141.6 mmol) was added, and subjected to the synthesis method of Sub 1-III-1 to give a product 13.97 g (yield: 70%).

(4) Synthesis of Sub 1-30

After Sub 1-III-30 (13.97 g, 39.7 mmol) obtained from the synthesis was dissolved in nitrobenzene (500 ml) in a round-bottom flask, iodobenzene (12.14 g, 59.5 mmol), Na₂SO₄ (5.63 g, 39.7 mmol), K₂CO₃ (5.48 g, 39.7 mmol), and Cu (0.76 g, 11.9 mmol) were added, and subjected to the synthesis method of Sub 1-1 to give a product 12.57 g (yield: 74%).

### 9. Synthesis Example of Sub 1-35

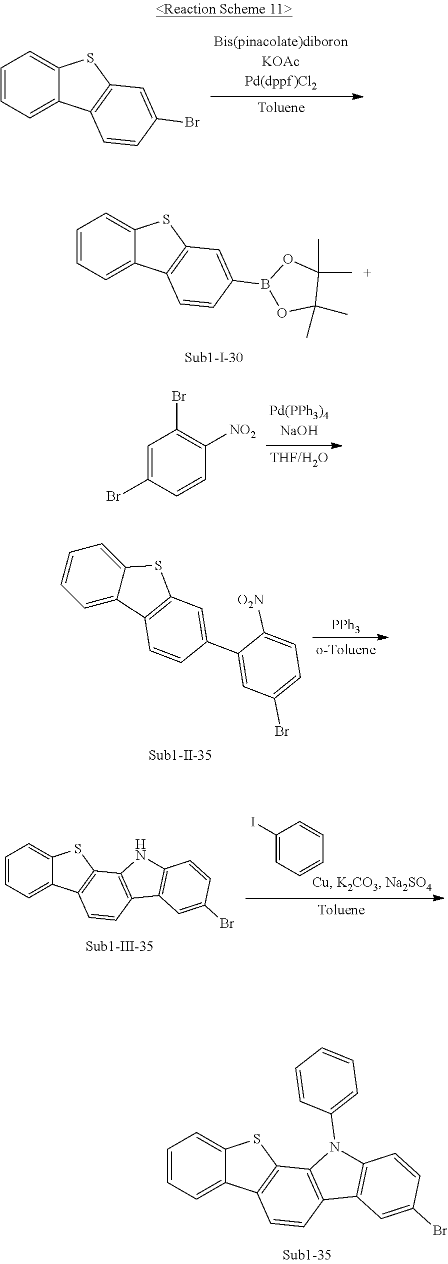

(1) Synthesis of Sub 1-II-35

After Sub 1-I-30 (19.81 g, 63.9 mmol) obtained from the synthesis was dissolved in THF (600 ml) in a round-bottom flask, 1,5-dibromo-2-nitrobenzene (26.91 g, 95.8 mmol), Pd(PPh₃)₄ (3.69 g, 3.2 mmol), K₂CO₃ (26.48 g, 191.6 mmol), and water (200 ml) were added, and subjected to the synthesis method of Sub 1-II-1 to give a product 18.89 g (yield: 77%).

(2) Synthesis of Sub 1-III-35

After Sub 1-II-35 (18.89 g, 49.2 mmol) obtained from the synthesis was dissolved in o-dichlorobenzene (246 ml) in a round-bottom flask, triphenylphosphine (32.24 g, 122.9 mmol) was added, and subjected to the synthesis method of Sub 1-III-1 to give a product 12.81 g (yield: 74%).

(3) Synthesis of Sub 1-35

After Sub 1-III-35 (12.81 g, 36.4 mmol) obtained from the synthesis was dissolved in nitrobenzene (450 ml) in a round-bottom flask, iodobenzene (11.13 g, 54.5 mmol), Na₂SO₄ (5.17 g, 36.4 mmol), K₂CO₃ (5.03 g, 36.4 mmol), and Cu (0.69 g, 10.9 mmol) were added, and subjected to the synthesis method of Sub 1-1 to give a product 11.68 g (yield: 75%).

### 10. Synthesis Example of Sub 1-39

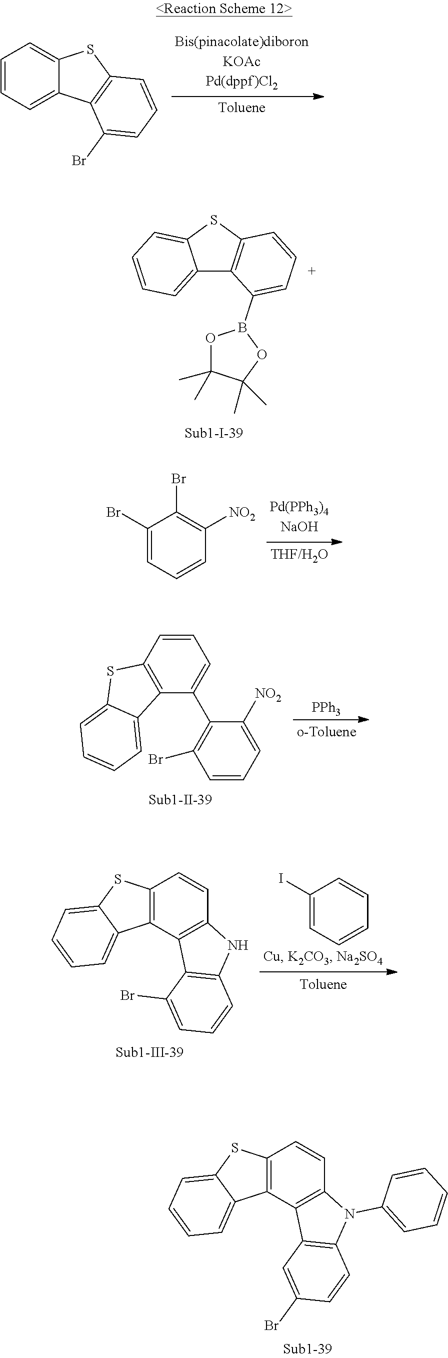

(1) Synthesis of Sub 1-I-39

1-Bromodibenzo[b,d]thiophene (43.00 g, 163.4 mmol), bis(pinacolato)diboron (45.64 g, 179.7 mmol), KOAc (48.11 g, 490.2 mmol), PdCl₂(dppf) (4.00 g, 4.9 mmol) and Toluene (800 mL) were added, and subjected to the synthesis method of Sub 1-I-1 to give a product 39.03 g (yield: 77%).

(2) Synthesis of Sub 1-II-39

After Sub 1-I-39 (39.03 g, 125.8 mmol) obtained from the synthesis was dissolved in THF (1200 ml) in a round-bottom flask 0-∥, 1,2-dibromo-3-nitrobenzene (53.01 g, 188.7 mmol), Pd(PPh₃)₄ (7.27 g, 6.3 mmol), K₂CO₃ (52.17 g, 377.4 mmol), and (300 ml) were added, and subjected to the synthesis method of Sub 1-II-1 to give a product 21.75 g (yield: 45%).

(3) Synthesis of Sub 1-III-39

After Sub 1-II-39 (21.75 g, 56.6 mmol) obtained from the synthesis was dissolved in o-dichlorobenzene (280 ml) in a round-bottom flask, triphenylphosphine (37.12 g, 141.5 mmol) was added, and subjected to the synthesis method of Sub 1-III-1 to give a product 12.56 g (yield: 63%).

(4) Synthesis of Sub 1-39

After Sub 1-III-39 (12.56 g, 35.7 mmol) obtained from the synthesis was dissolved in nitrobenzene (440 ml) in a round-bottom flask, iodobenzene (10.91 g, 53.5 mmol), Na₂SO₄ (5.06 g, 35.7 mmol), K₂CO₃ (4.93 g, 35.7 mmol), and Cu (0.68 g, 10.7 mmol) were added, and subjected to the synthesis method of Sub 1-1 to give a product 10.39 g (yield: 68%).

### 11. Synthesis Example of Sub 1-44

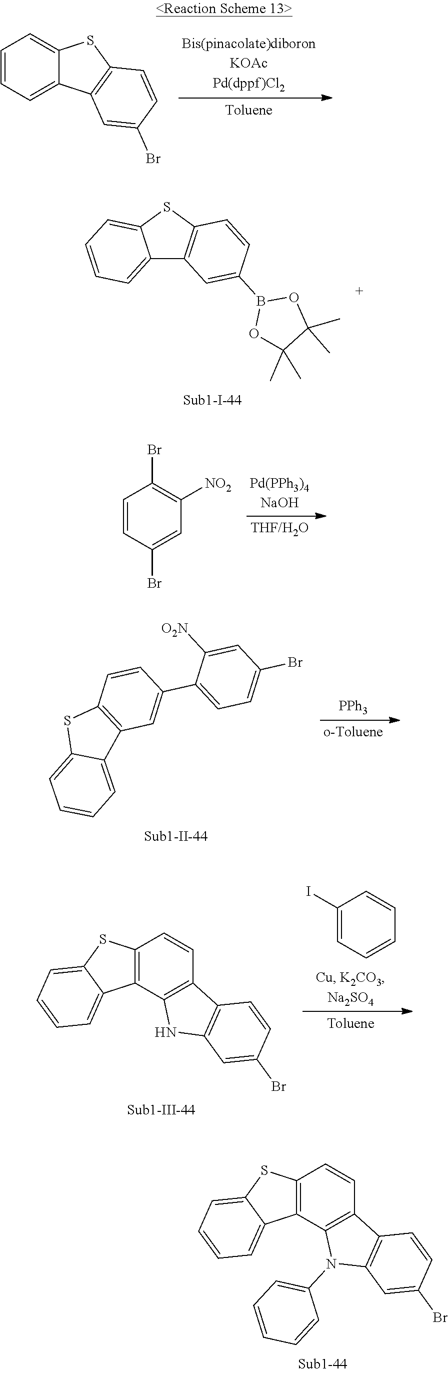

(1) Synthesis of Sub 1-I-44

2-bromodibenzo[b,d]thiophene (50.00 g, 190.0 mmol), bis(pinacolato)diboron (53.08 g, 209.0 mmol), KOAc (55.94 g, 570.0 mmol), PdCl₂(dppf) (4.65 g, 5.7 mmol) and Toluene (950 mL) were added, and subjected to the synthesis method of Sub 1-I-1 to give a product 45.98 g (yield: 78%).

(2) Synthesis of Sub 1-II-44

After Sub 1-I-44 (18.39 g, 59.3 mmol) obtained from the synthesis was dissolved in THF (600 ml) in a round-bottom flask 0-∥, 1,4-dibromo-2-nitrobenzene (24.98 g, 88.9 mmol), Pd(PPh₃)₄ (3.43 g, 3.0 mmol), K₂CO₃ (24.58 g, 177.8 mmol), and water (150 ml) were added, and subjected to the synthesis method of Sub 1-II-1 to give a product 18.45 g (yield: 81%).

(3) Synthesis of Sub 1-III-44

After Sub 1-II-44 (18.45 g, 48.0 mmol) obtained from the synthesis was dissolved in o-dichlorobenzene (240 ml) in a round-bottom flask, triphenylphosphine (31.49 g, 120.0 mmol) was added, and subjected to the synthesis method of Sub 1-III-1 to give a product 11.84 g (yield: 70%).

(4) Synthesis of Sub 1-44

After Sub 1-III-44 (11.84 g, 33.6 mmol) obtained from the synthesis was dissolved in nitrobenzene (420 ml) in a round-bottom flask, iodobenzene (10.29 g, 50.4 mmol), Na₂SO₄ (4.77 g, 33.6 mmol), K₂CO₃ (4.65 g, 33.6 mmol), and Cu (0.64 g, 10.1 mmol) were added, and subjected to the synthesis method of Sub 1-1 to give a product 10.08 g (yield: 70%).

### 12. Synthesis Example of Sub 1-47

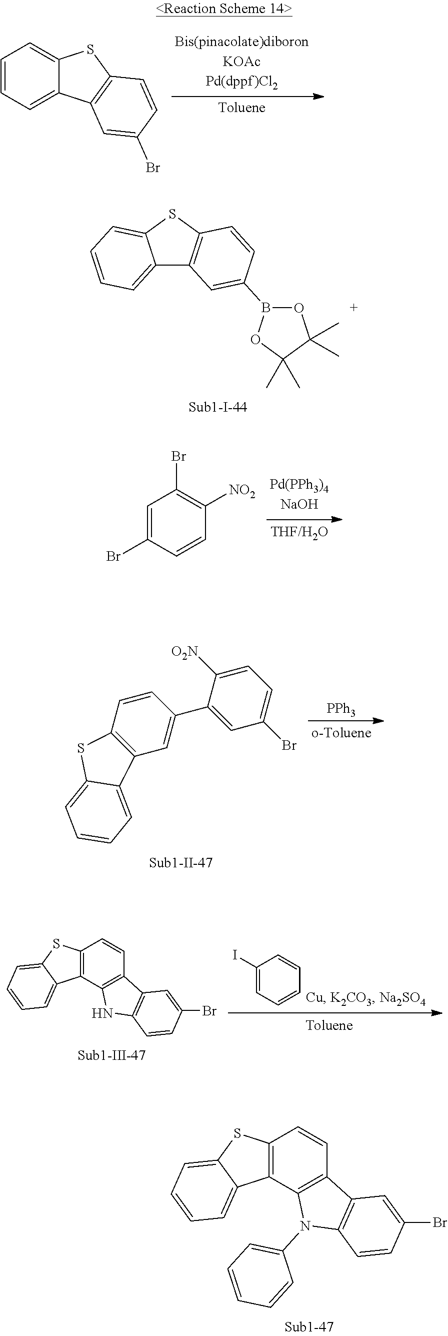

Synthesis of Sub 1-II-47

After Sub 1-I-44 (25.35 g, 81.7 mmol) obtained from the synthesis was dissolved in THF (800 ml) in a round-bottom flask, 1,5-dibromo-2-nitrobenzene (34.43 g, 122.6 mmol), Pd(PPh₃)₄ (4.72 g, 4.1 mmol), K₂CO₃ (33.88 g, 245.1 mmol), and water (200 ml) were added, and subjected to the synthesis method of Sub 1-II-1 to give a product 22.61 g (yield: 72%).

(2) Synthesis of Sub 1-III-47

After Sub 1-II-47 (22.61 g, 58.8 mmol) obtained from the synthesis was dissolved in o-dichlorobenzene (300 ml) in a round-bottom flask, triphenylphosphine (38.58 g, 147.1 mmol) was added, and subjected to the synthesis method of Sub 1-III-1 to give a product 13.89 g (yield: 67%).

(3) Synthesis of Sub 1-47

After Sub 1-III-47 (13.89 g, 39.4 mmol) obtained from the synthesis was dissolved in nitrobenzene (500 ml) in a round-bottom flask, iodobenzene (12.07 g, 59.1 mmol), Na₂SO₄ (5.60 g, 39.4 mmol), K₂CO₃ (5.45 g, 39.4 mmol), and Cu (0.75 g, 11.8 mmol) were added, and subjected to the synthesis method of Sub 1-1 to give a product 12.33 g (yield: 73%).

### 13. Synthesis Example of Sub 1-56

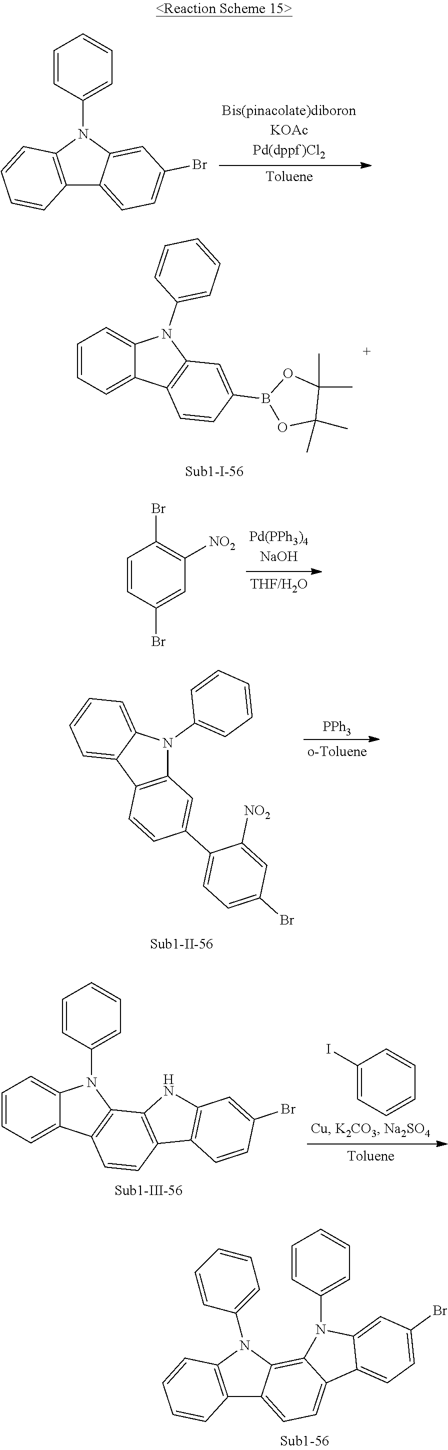

(1) Synthesis of Sub 1-I-56

2-Bromo-9-phenyl-9H-carbazole (40.00 g, 124.1 mmol), bis(pinacolato)diboron (34.68 g, 136.6 mmol), KOAc (36.55 g, 372.4 mmol), PdCl₂(dppf) (3.04 g, 3.7 mmol), and Toluene (620 mL) were added, and subjected to the synthesis method of Sub 1-I-1 to give a product 36.22 g (yield: 79%).

(2) Synthesis of Sub 1-II-56

After Sub 1-I-56 (36.22 g, 98.1 mmol) obtained from the synthesis was dissolved in THF (900 ml) in a round-bottom flask, 1,4-dibromo-2-nitrobenzene (41.33 g, 147.1 mmol), Pd(PPh₃)₄ (5.67 g, 4.9 mmol), K₂CO₃ (40.67 g, 294.3 mmol), and water (300 ml) were added, and subjected to the synthesis method of Sub 1-II-1 to give a product 31.31 g (yield: 72%).

(3) Synthesis of Sub 1-III-56

After Sub 1-II-56 (31.31 g, 70.6 mmol) obtained from the synthesis was dissolved in o-dichlorobenzene (410 ml) in a round-bottom flask, triphenylphosphine (46.31 g, 176.6 mmol) was added, and subjected to the synthesis method of Sub 1-III-1 to give a product 20.33 g (yield: 70%).

(4) Synthesis of Sub 1-56

After Sub 1-III-56 (20.33 g, 49.4 mmol) obtained from the synthesis was dissolved in nitrobenzene (620 ml) in a round-bottom flask, iodobenzene (15.13 g, 74.1 mmol), Na₂SO₄ (7.02 g, 49.4 mmol), K₂CO₃ (6.83 g, 49.4 mmol), and Cu (0.94 g, 14.8 mmol) were added, and subjected to the synthesis method of Sub 1-1 to give a product 18.07 g (yield: 75%).

### 14. Synthesis Example of Sub 1-57

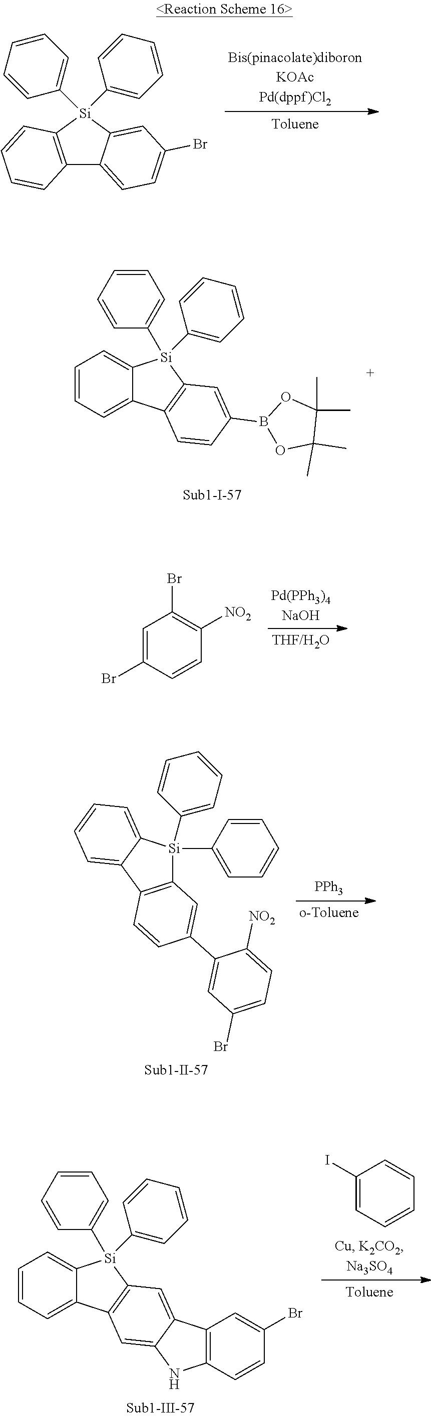

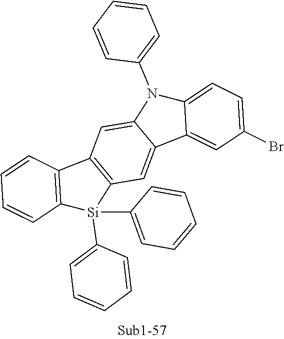 [SMILES: Brc1ccc2c(c1)c1cc3c(cc1n2-c1ccccc1)-c1ccccc1[Si]3(c1ccccc1)c1ccccc1]

(1) Synthesis of Sub 1-I-57

After 3-bromo-5,5-diphenyl-5H-dibenzo[b,d]silole (50.00 g, 121.0 mmol), bis(pinacolato)diboron (33.79 g, 133.0 mmol), KOAc (35.61 g, 362.9 mmol), and PdCl₂(dppf) (2.96 g, 3.6 mmol) were dissolved in Toluene (605 mL) solvent, and refluxed at 120° C. for 12 hours. Upon the completion of the reaction, the reaction product was cooled to normal temperature, extracted with CH₂Cl₂, and washed with water. The organic layer was dried over MgSO₄ and concentrated, and then the formed organic material was recrystallized using CH₂Cl₂ and methanol solvents to obtain a desired product (39.54 g, 71%).

(2) Synthesis of Sub 1-II-57

After Sub 1-I-57 (39.54 g, 76.0 mmol) obtained from the synthesis was dissolved in THF (800 ml) in a round-bottom flask, 1,5-dibromo-2-nitrobenzene (32.03 g, 114.0 mmol), Pd(PPh₃)₄ (4.39 g, 3.8 mmol), K₂CO₃ (31.52 g, 228.0 mmol), and water (150 ml) were added, and subjected to the synthesis method of Sub 1-II-1 to give a product 40.62 g (yield: 75%).

(3) Synthesis of Sub 1-III-57

After Sub 1-II-57 (30.0 g, 56.1 mmol) obtained from the synthesis was dissolved in o-dichlorobenzene (280 ml) in a round-bottom flask, triphenylphosphine (36.81 g, 140.3 mmol) was added, and subjected to the synthesis method of Sub 1-III-1 to give a product 12.13 g (yield: 43%).

(4) Synthesis of Sub 1-57

After Sub 1-III-57 (12.13 g, 24.1 mmol) obtained from the synthesis was dissolved in nitrobenzene (300 ml) in a round-bottom flask, iodobenzene (7.39 g, 36.2 mmol), Na₂SO₄ (3.43 g, 24.1 mmol), K₂CO₃ (3.34 g, 24.1 mmol), and Cu (0.46 g, 7.2 mmol) were added, and subjected to the synthesis method of Sub 1-1 to give a product 9.07 g (yield: 65%).

Meanwhile, the compounds pertaining to Sub 1 may be compounds below, but are not limited thereto. Table 1 below shows FD-MS values of the compounds pertaining to Sub 1.

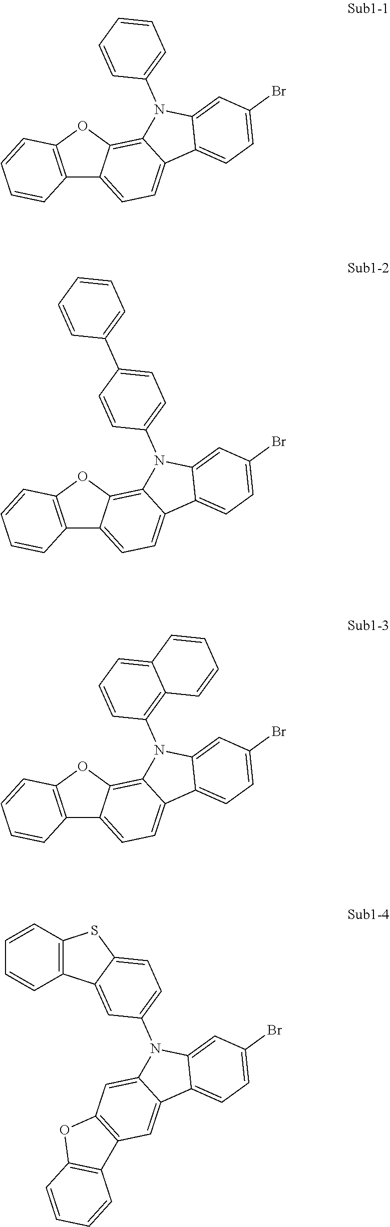 [SMILES: Brc1ccc2c3ccc4c5ccccc5oc4c3n(-c3ccccc3)c2c1 ; Brc1ccc2c3ccc4c5ccccc5oc4c3n(-c3ccc(-c4ccccc4)cc3)c2c1 ; Brc1ccc2c3ccc4c5ccccc5oc4c3n(-c3cccc4ccccc34)c2c1 ; Brc1ccc2c3cc4c(cc3n(-c3ccc5sc6ccccc6c5c3)c2c1)oc1ccccc14]

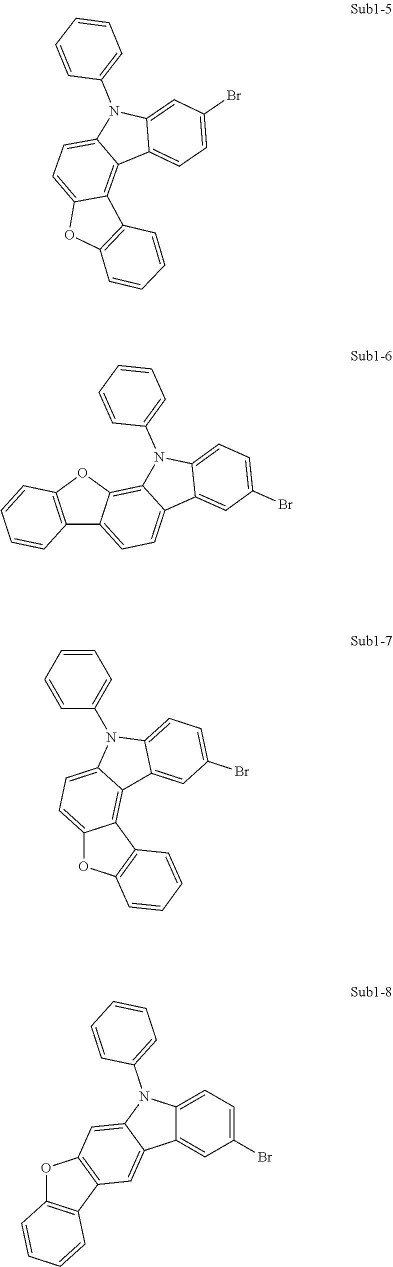 [SMILES: Brc1ccc2c3c4c(ccc3n(-c3ccccc3)c2c1)oc1ccccc14 ; Brc1ccc2c(c1)c1ccc3c4ccccc4oc3c1n2-c1ccccc1 ; Brc1ccc2c(c1)c1c3c(ccc1n2-c1ccccc1)oc1ccccc13 ; Brc1ccc2c(c1)c1cc3c(cc1n2-c1ccccc1)oc1ccccc13]

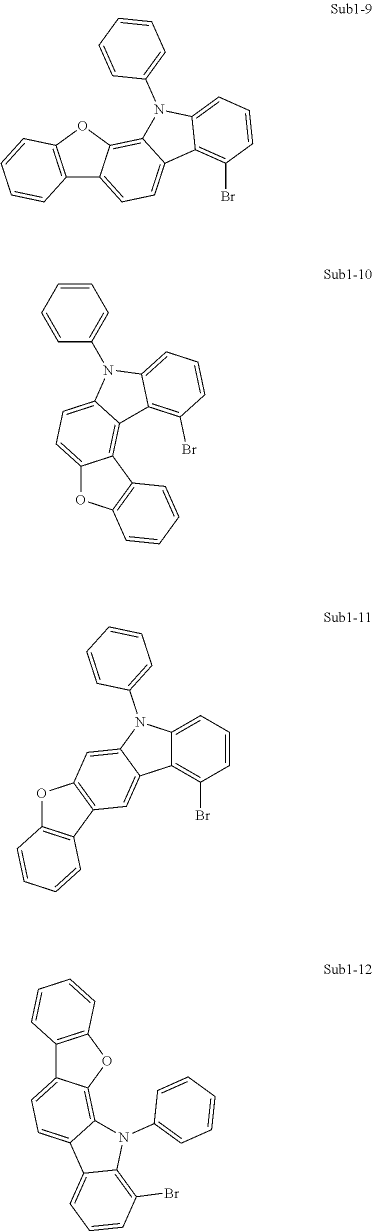 [SMILES: Brc1cccc2c1c1ccc3c4ccccc4oc3c1n2-c1ccccc1 ; Brc1cccc2c1c1c3c(ccc1n2-c1ccccc1)oc1ccccc13 ; Brc1cccc2c1c1cc3c(cc1n2-c1ccccc1)oc1ccccc13 ; Brc1cccc2c3ccc4c5ccccc5oc4c3n(-c3ccccc3)c12]

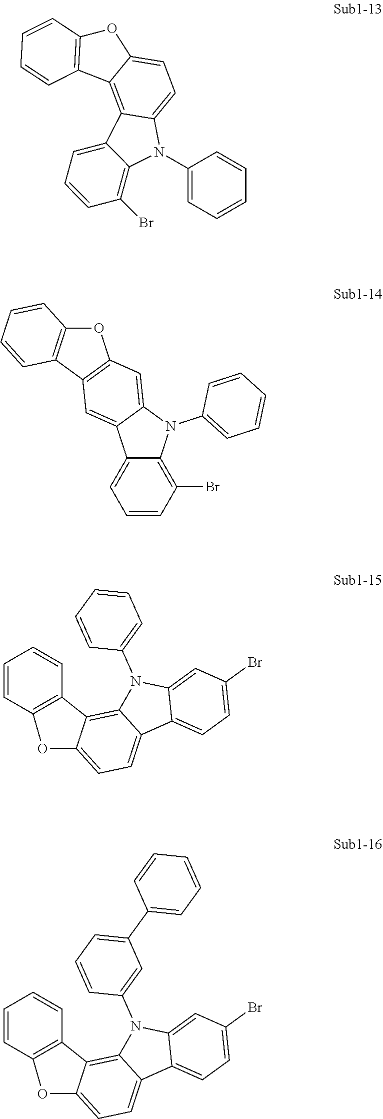 [SMILES: Brc1cccc2c3c4c(ccc3n(-c3ccccc3)c12)oc1ccccc14 ; Brc1cccc2c3cc4c(cc3n(-c3ccccc3)c12)oc1ccccc14 ; Brc1ccc2c3ccc4oc5ccccc5c4c3n(-c3ccccc3)c2c1 ; Brc1ccc2c3ccc4oc5ccccc5c4c3n(-c3cccc(-c4ccccc4)c3)c2c1]

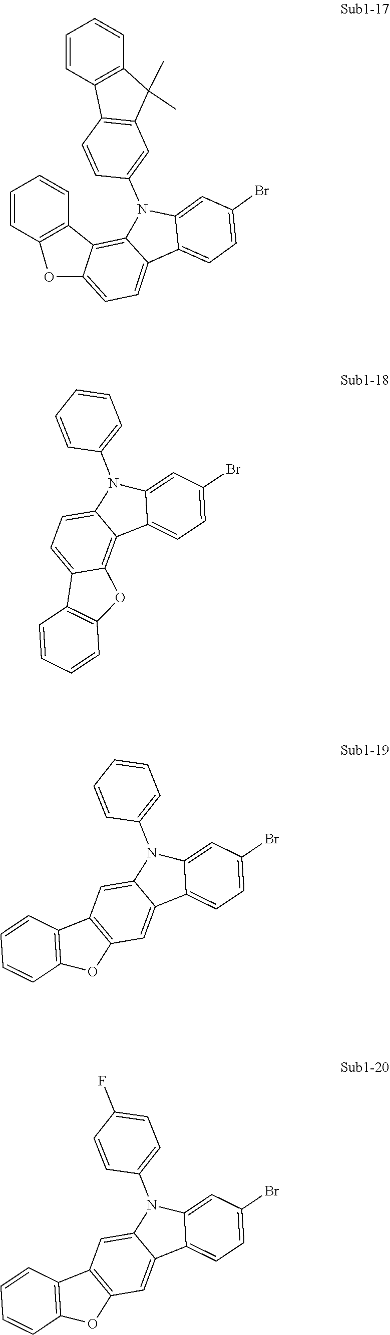 [SMILES: CC1(C)c2ccccc2-c2ccc(-n3c4cc(Br)ccc4c4ccc5oc6ccccc6c5c43)cc21 ; Brc1ccc2c3c4oc5ccccc5c4ccc3n(-c3ccccc3)c2c1 ; Brc1ccc2c3cc4oc5ccccc5c4cc3n(-c3ccccc3)c2c1 ; Fc1ccc(-n2c3cc(Br)ccc3c3cc4oc5ccccc5c4cc32)cc1]

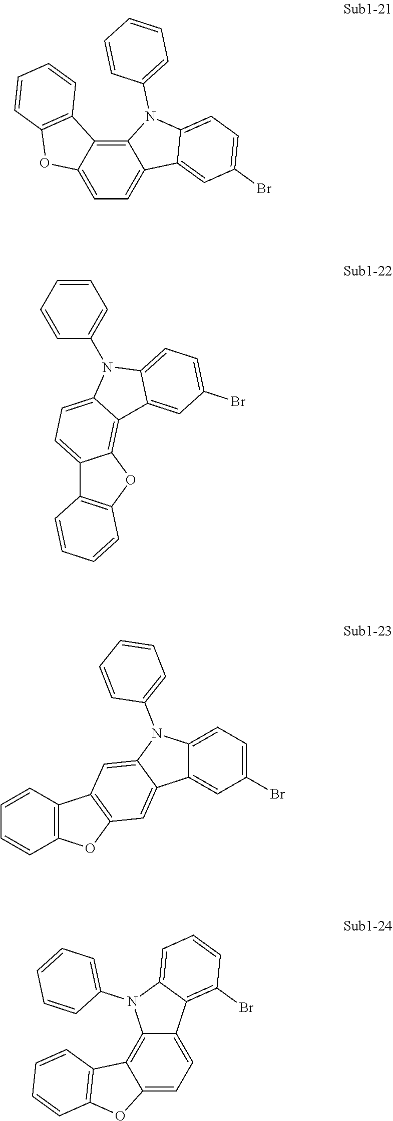 [SMILES: Brc1ccc2c(c1)c1ccc3oc4ccccc4c3c1n2-c1ccccc1 ; Brc1ccc2c(c1)c1c3oc4ccccc4c3ccc1n2-c1ccccc1 ; Brc1ccc2c(c1)c1cc3oc4ccccc4c3cc1n2-c1ccccc1 ; Brc1cccc2c1c1ccc3oc4ccccc4c3c1n2-c1ccccc1]

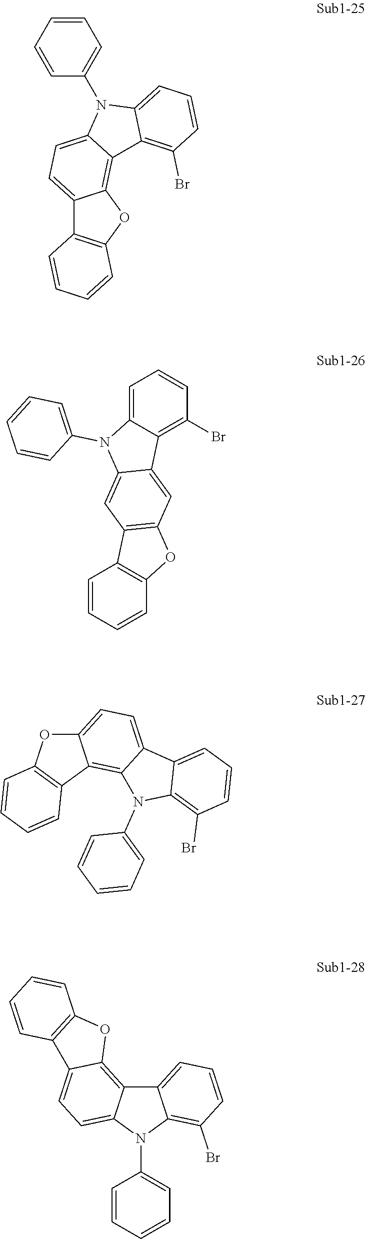 [SMILES: Brc1cccc2c1c1c3oc4ccccc4c3ccc1n2-c1ccccc1 ; Brc1cccc2c1c1cc3oc4ccccc4c3cc1n2-c1ccccc1 ; Brc1cccc2c3ccc4oc5ccccc5c4c3n(-c3ccccc3)c12 ; Brc1cccc2c3c4oc5ccccc5c4ccc3n(-c3ccccc3)c12]

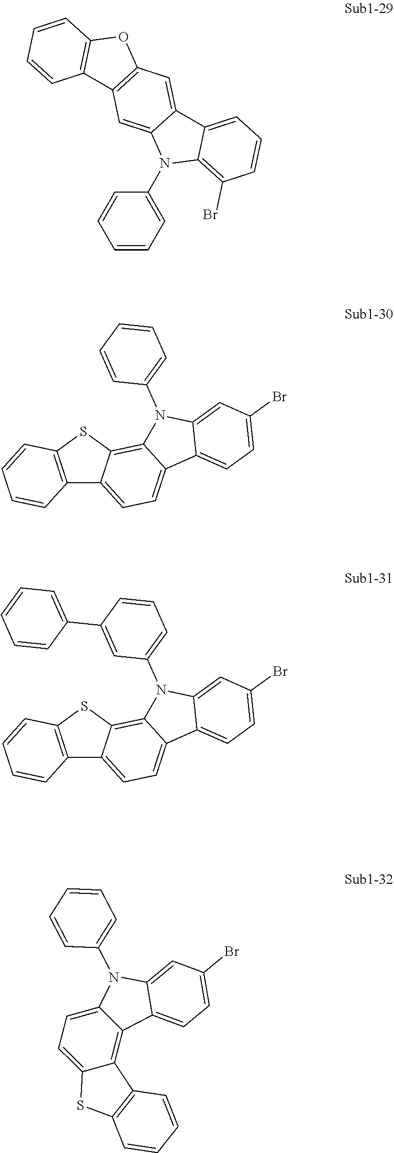 [SMILES: Brc1cccc2c3cc4oc5ccccc5c4cc3n(-c3ccccc3)c12 ; Brc1ccc2c3ccc4c5ccccc5sc4c3n(-c3ccccc3)c2c1 ; Brc1ccc2c3ccc4c5ccccc5sc4c3n(-c3cccc(-c4ccccc4)c3)c2c1 ; Brc1ccc2c3c4c(ccc3n(-c3ccccc3)c2c1)sc1ccccc14]

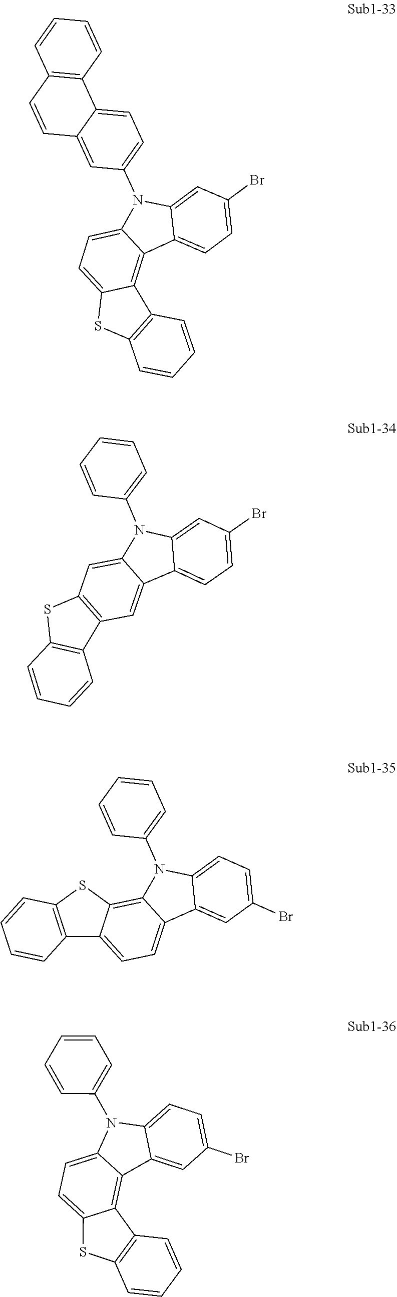 [SMILES: Brc1ccc2c3c4c(ccc3n(-c3ccc5c(ccc6ccccc65)c3)c2c1)sc1ccccc14 ; Brc1ccc2c3cc4c(cc3n(-c3ccccc3)c2c1)sc1ccccc14 ; Brc1ccc2c(c1)c1ccc3c4ccccc4sc3c1n2-c1ccccc1 ; Brc1ccc2c(c1)c1c3c(ccc1n2-c1ccccc1)sc1ccccc13]

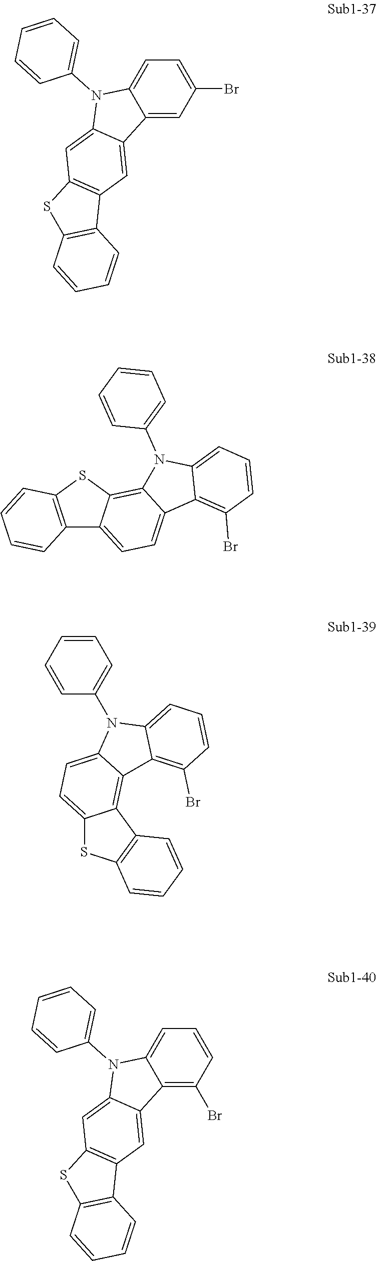 [SMILES: Brc1ccc2c(c1)c1cc3c(cc1n2-c1ccccc1)sc1ccccc13 ; Brc1cccc2c1c1ccc3c4ccccc4sc3c1n2-c1ccccc1 ; Brc1cccc2c1c1c3c(ccc1n2-c1ccccc1)sc1ccccc13 ; Brc1cccc2c1c1cc3c(cc1n2-c1ccccc1)sc1ccccc13]

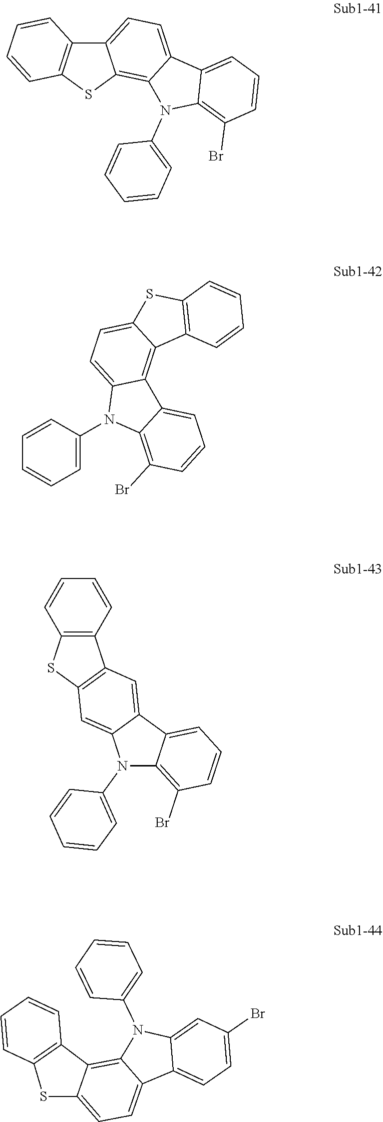 [SMILES: Brc1cccc2c3ccc4c5ccccc5sc4c3n(-c3ccccc3)c12 ; Brc1cccc2c3c4c(ccc3n(-c3ccccc3)c12)sc1ccccc14 ; Brc1cccc2c3cc4c(cc3n(-c3ccccc3)c12)sc1ccccc14 ; Brc1ccc2c3ccc4sc5ccccc5c4c3n(-c3ccccc3)c2c1]

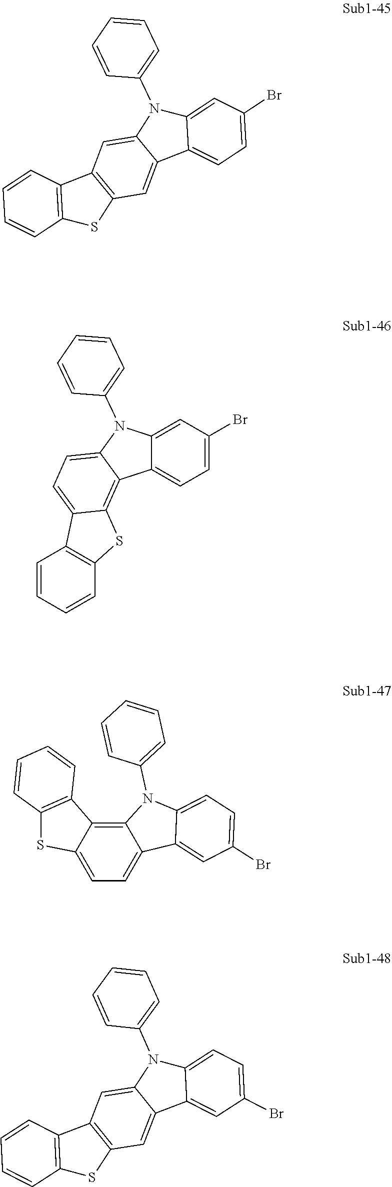 [SMILES: Brc1ccc2c3cc4sc5ccccc5c4cc3n(-c3ccccc3)c2c1 ; Brc1ccc2c3c4sc5ccccc5c4ccc3n(-c3ccccc3)c2c1 ; Brc1ccc2c(c1)c1ccc3sc4ccccc4c3c1n2-c1ccccc1 ; Brc1ccc2c(c1)c1cc3sc4ccccc4c3cc1n2-c1ccccc1]

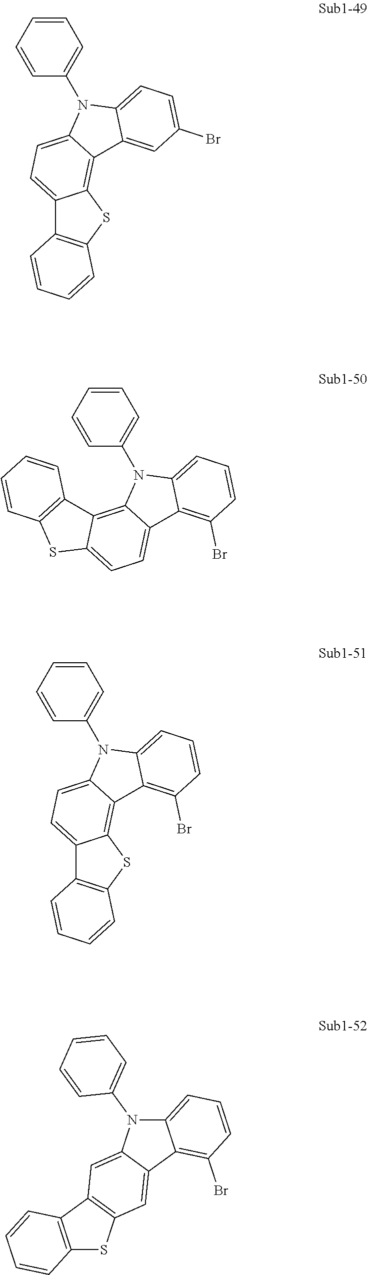 [SMILES: Brc1ccc2c(c1)c1c3sc4ccccc4c3ccc1n2-c1ccccc1 ; Brc1cccc2c1c1ccc3sc4ccccc4c3c1n2-c1ccccc1 ; Brc1cccc2c1c1c3sc4ccccc4c3ccc1n2-c1ccccc1 ; Brc1cccc2c1c1cc3sc4ccccc4c3cc1n2-c1ccccc1]

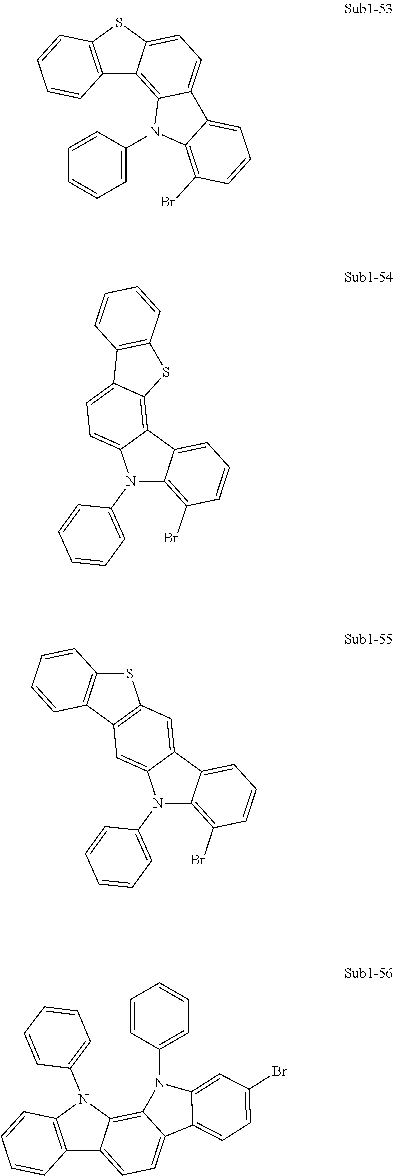 [SMILES: Brc1cccc2c3ccc4sc5ccccc5c4c3n(-c3ccccc3)c12 ; Brc1cccc2c3c4sc5ccccc5c4ccc3n(-c3ccccc3)c12 ; Brc1cccc2c3cc4sc5ccccc5c4cc3n(-c3ccccc3)c12 ; Brc1ccc2c3ccc4c5ccccc5n(-c5ccccc5)c4c3n(-c3ccccc3)c2c1]

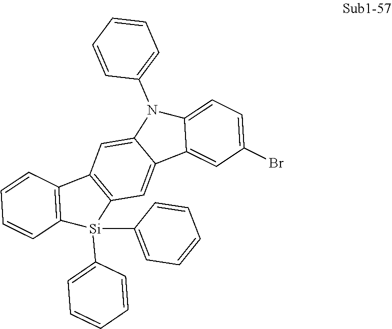 [SMILES: Brc1ccc2c(c1)c1cc3c(cc1n2-c1ccccc1)-c1ccccc1[Si]3(c1ccccc1)c1ccccc1]

TABLE 1

| Compound | FD-MS | Compound | FD-MS |
|---|---|---|---|
| Sub1-1 | m/z = 411.03(C₂₄H₁₄BrNO = 412.29) | Sub1-4 | m/z = 517.01(C30H16BrNOS = 518.43) |
| Sub1-7 | m/z = 411.03(C₂₄H₁₄BrNO = 412.29) | Sub1-9 | m/z = 411.03(C₂₄H₁₄BrNO = 412.29) |
| Sub1-13 | m/z = 411.03(C₂₄H₁₄BrNO = 412.29) | Sub1-18 | m/z = 411.03(C₂₄H₁₄BrNO = 412.29) |
| Sub1-26 | m/z = 411.03(C₂₄H₁₄BrNO = 412.29) | Sub1-30 | m/z = 427.00(C₂₄H₁₄BrNS = 428.35) |
| Sub1-35 | m/z = 427.00(C₂₄H₁₄BrNS = 428.35) | Sub1-39 | m/z = 427.00(C₂₄H₁₄BrNS = 428.35) |
| Sub1-44 | m/z = 427.00(C₂₄H₁₄BrNS = 428.35) | Sub1-47 | m/z = 427.00(C₂₄H₁₄BrNS = 428.35) |
| Sub1-56 | m/z = 486.07(C₃₀H₁₉BrN₂ = 487.40) | Sub1-57 | m/z = 577.09(C₃₆H₂₄BrNSi = 578.58) |

II. Synthesis of Sub 2

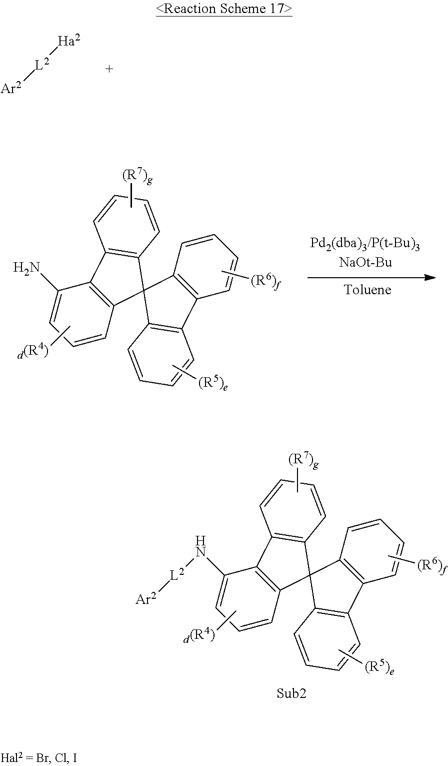

Synthesis examples of specific compounds pertaining to Sub 2 are as follows.

### 1. Synthesis Example of Sub 2-1

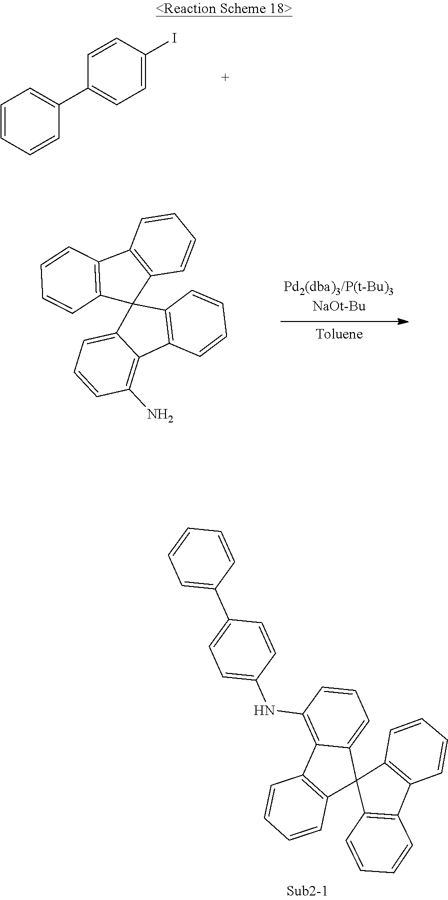 [SMILES: Ic1ccc(-c2ccccc2)cc1 ; Nc1cccc2c1-c1ccccc1C21c2ccccc2-c2ccccc21 ; c1ccc(-c2ccc(Nc3cccc4c3-c3ccccc3C43c4ccccc4-c4ccccc43)cc2)cc1]

After the start material 4-iodo-1,1′-biphenyl (15.00 g, 53.6 mmol) was dissolved in toluene (669 ml) in a round-bottom flask, 9,9′-spirobi[fluoren]-4-amine (26.62 g, 80.3 mmol), Pd₂(dba)₃ (1.47 g, 1.6 mmol), 50% P(t-Bu)₃ (1.6 ml, 3.2 mmol), and NaOt-Bu (15.44 g, 160.7 mmol) were added, followed by stirring at 40° C. Upon completion of the reaction, the reaction product was extracted with CH₂Cl₂ and water. The organic layer was dried over MgSO₄ and concentrated, and then the formed compound was subjected to silica gel column and recrystallization to give a product 20.46 g (yield: 79%).

### 2. Synthesis Example of Sub 2-4

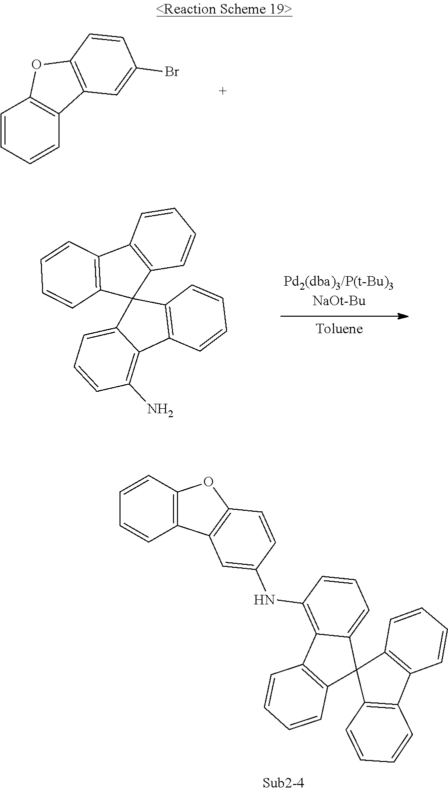 [SMILES: Brc1ccc2oc3ccccc3c2c1 ; Nc1cccc2c1-c1ccccc1C21c2ccccc2-c2ccccc21 ; c1ccc2c(c1)-c1ccccc1C21c2ccccc2-c2c(Nc3ccc4oc5ccccc5c4c3)cccc21]

After the start material 2-bromodibenzo[b,d]furan (10.00 g, 40.5 mmol) was dissolved in toluene (506 ml) in a round-bottom flask, 9,9′-spirobi[fluoren]-4-amine (20.12 g, 60.7 mmol), Pd₂(dba)₃ (1.11 g, 1.2 mmol), 50% P(t-Bu)₃ (1.2 ml, 2.4 mmol), and NaOt-Bu (11.67 g, 121.4 mmol) were added, and subjected to the synthesis method of Sub 2-1 to give a product 15.10 g (yield: 75%).

### 3. Synthesis Example of Sub 2-6

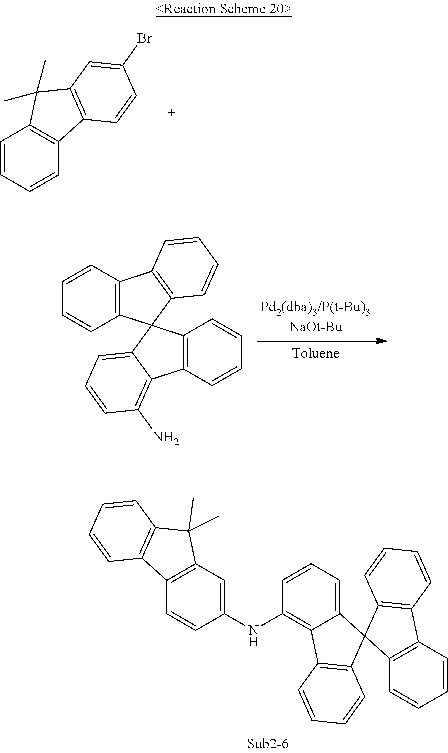 [SMILES: CC1(C)c2ccccc2-c2ccc(Br)cc21 ; Nc1cccc2c1-c1ccccc1C21c2ccccc2-c2ccccc21 ; CC1(C)c2ccccc2-c2ccc(Nc3cccc4c3-c3ccccc3C43c4ccccc4-c4ccccc43)cc21]

After the start material 2-bromo-9,9-dimethyl-9H-fluorene (15.00 g, 54.9 mmol) was dissolved in toluene (686 ml) in a round-bottom flask, 9,9′-spirobi[fluorene]-4-amine (27.30 g, 82.4 mmol), Pd₂(dba)₃ (1.51 g, 1.6 mmol), 50% P(t-Bu)₃ (1.6 ml, 3.3 mmol), and NaOt-Bu (15.83 g, 164.7 mmol) were added, and subjected to the synthesis method of Sub 2-1 to give a product 20.42 g (yield: 71%).

Meanwhile, the compounds pertaining to Sub 2 may be compounds below, but are not limited thereto. Table 2 below shows FD-MS values of the compounds pertaining to Sub 2.

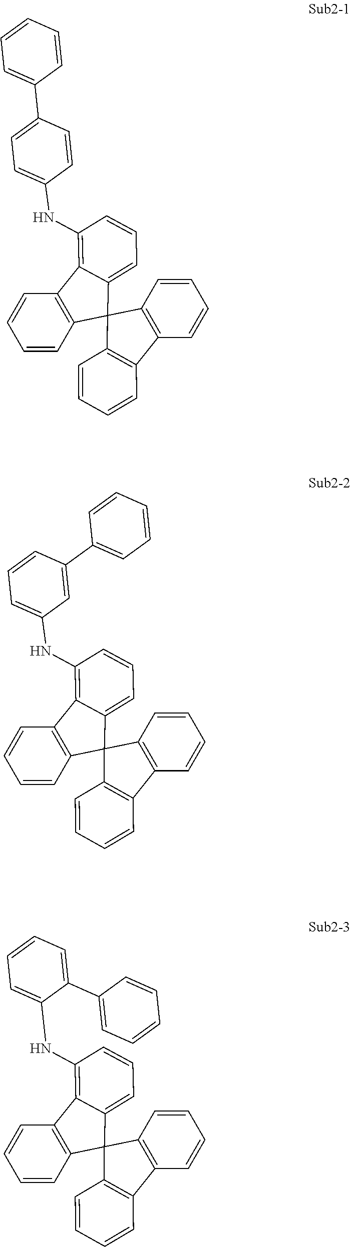 [SMILES: c1ccc(-c2ccc(Nc3cccc4c3-c3ccccc3C43c4ccccc4-c4ccccc43)cc2)cc1 ; c1ccc(-c2cccc(Nc3cccc4c3-c3ccccc3C43c4ccccc4-c4ccccc43)c2)cc1 ; c1ccc(-c2ccccc2Nc2cccc3c2-c2ccccc2C32c3ccccc3-c3ccccc32)cc1]

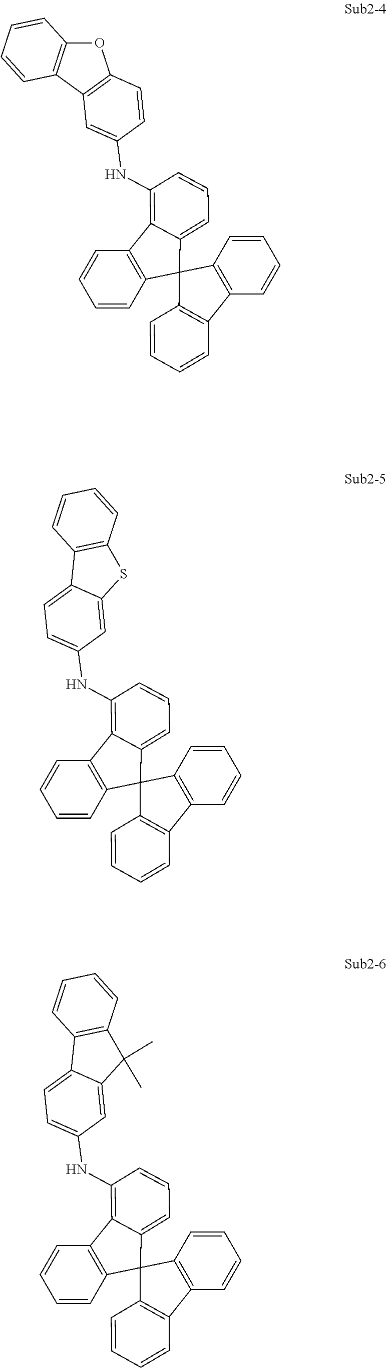 [SMILES: c1ccc2c(c1)-c1ccccc1C21c2ccccc2-c2c(Nc3ccc4oc5ccccc5c4c3)cccc21 ; c1ccc2c(c1)-c1ccccc1C21c2ccccc2-c2c(Nc3ccc4c(c3)sc3ccccc34)cccc21 ; CC1(C)c2ccccc2-c2ccc(Nc3cccc4c3-c3ccccc3C43c4ccccc4-c4ccccc43)cc21]

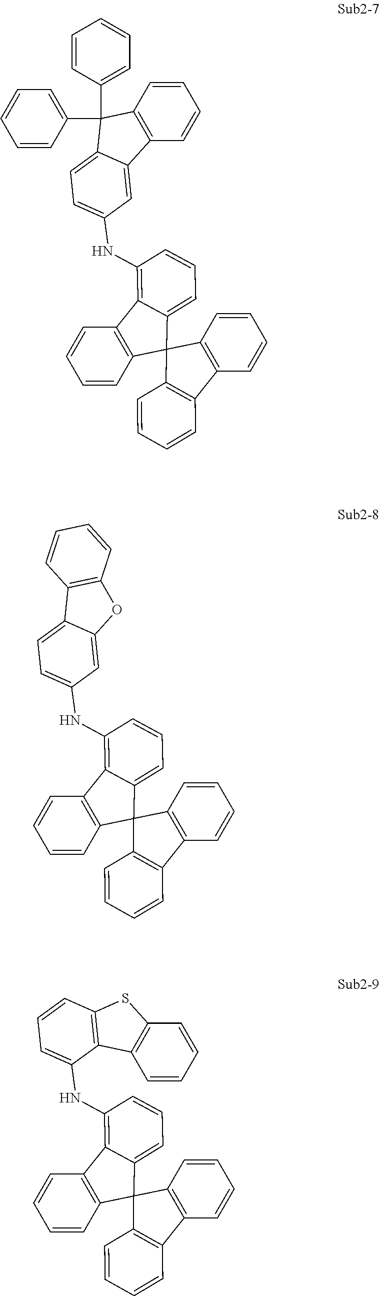 [SMILES: c1ccc(C2(c3ccccc3)c3ccccc3-c3cc(Nc4cccc5c4-c4ccccc4C54c5ccccc5-c5ccccc54)ccc32)cc1 ; c1ccc2c(c1)-c1ccccc1C21c2ccccc2-c2c(Nc3ccc4c(c3)oc3ccccc34)cccc21 ; c1ccc2c(c1)-c1ccccc1C21c2ccccc2-c2c(Nc3cccc4sc5ccccc5c34)cccc21]

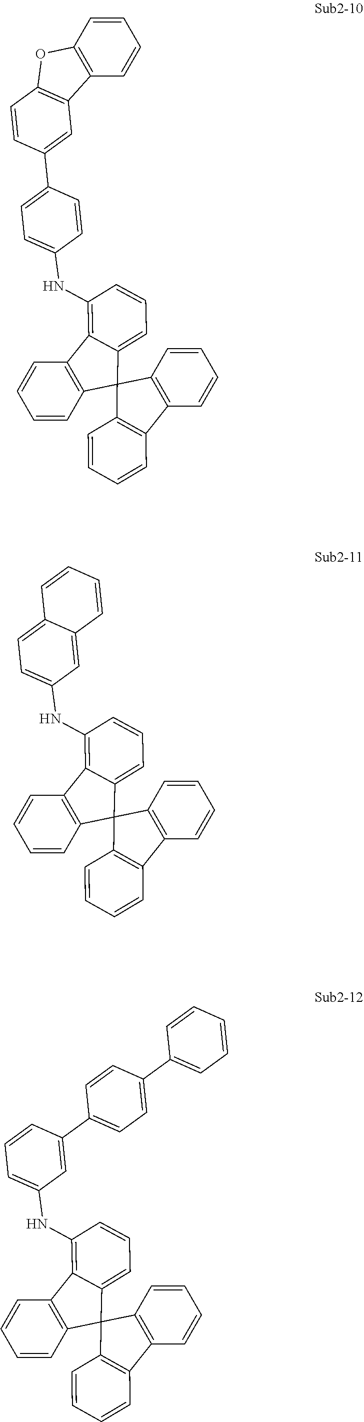 [SMILES: c1ccc2c(c1)-c1ccccc1C21c2ccccc2-c2c(Nc3ccc(-c4ccc5oc6ccccc6c5c4)cc3)cccc21 ; c1ccc2c(c1)-c1ccccc1C21c2ccccc2-c2c(Nc3ccc4ccccc4c3)cccc21 ; c1ccc(-c2ccc(-c3cccc(Nc4cccc5c4-c4ccccc4C54c5ccccc5-c5ccccc54)c3)cc2)cc1]

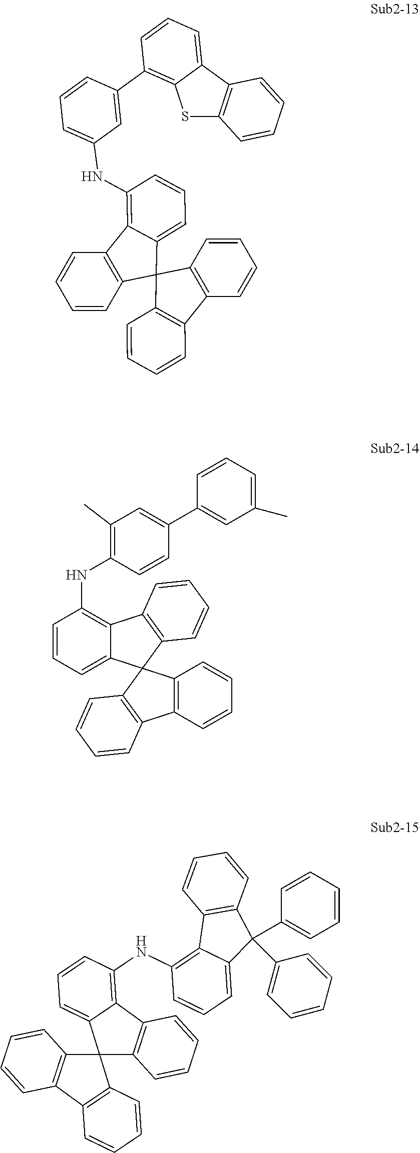 [SMILES: c1cc(Nc2cccc3c2-c2ccccc2C32c3ccccc3-c3ccccc32)cc(-c2cccc3c2sc2ccccc23)c1 ; Cc1cccc(-c2ccc(Nc3cccc4c3-c3ccccc3C43c4ccccc4-c4ccccc43)c(C)c2)c1 ; c1ccc(C2(c3ccccc3)c3ccccc3-c3c(Nc4cccc5c4-c4ccccc4C54c5ccccc5-c5ccccc54)cccc32)cc1]

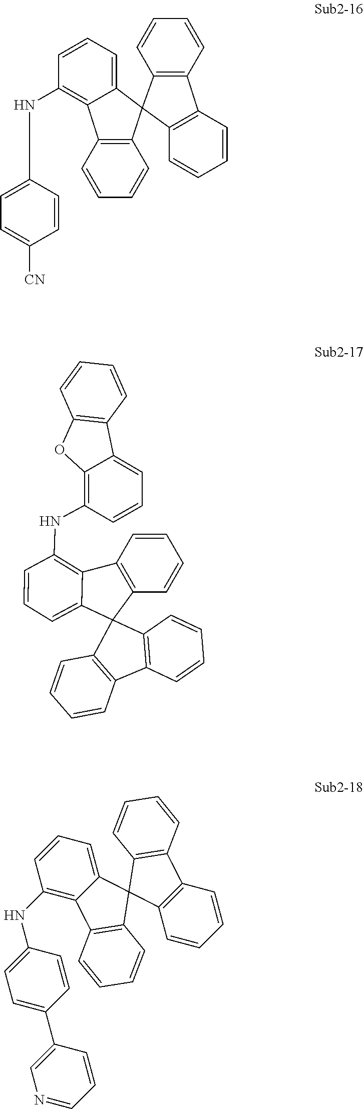 [SMILES: N#Cc1ccc(Nc2cccc3c2-c2ccccc2C32c3ccccc3-c3ccccc32)cc1 ; c1ccc2c(c1)-c1ccccc1C21c2ccccc2-c2c(Nc3cccc4c3oc3ccccc34)cccc21 ; c1cncc(-c2ccc(Nc3cccc4c3-c3ccccc3C43c4ccccc4-c4ccccc43)cc2)c1]

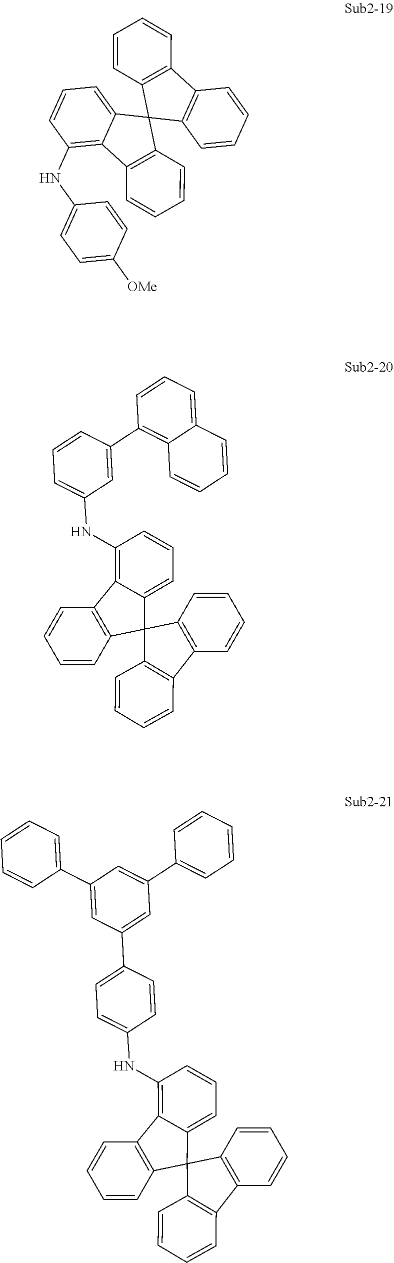 [SMILES: COc1ccc(Nc2cccc3c2-c2ccccc2C32c3ccccc3-c3ccccc32)cc1 ; c1cc(Nc2cccc3c2-c2ccccc2C32c3ccccc3-c3ccccc32)cc(-c2cccc3ccccc23)c1 ; c1ccc(-c2cc(-c3ccccc3)cc(-c3ccc(Nc4cccc5c4-c4ccccc4C54c5ccccc5-c5ccccc54)cc3)c2)cc1]

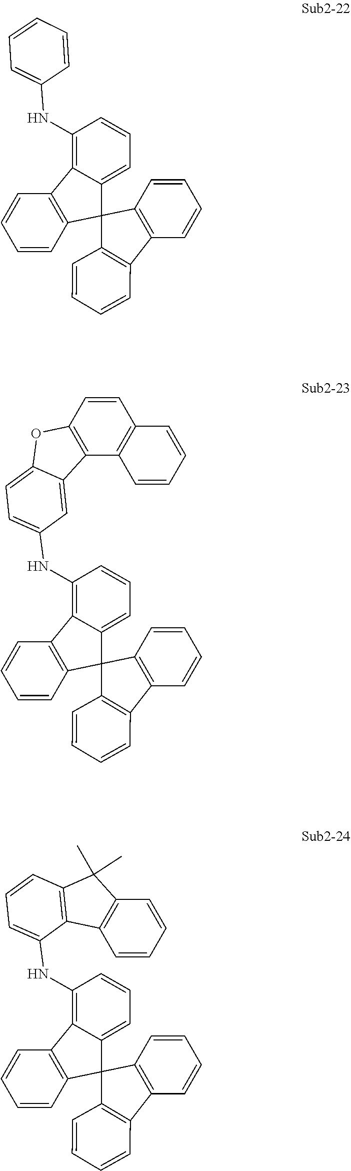 [SMILES: c1ccc(Nc2cccc3c2-c2ccccc2C32c3ccccc3-c3ccccc32)cc1 ; c1ccc2c(c1)-c1ccccc1C21c2ccccc2-c2c(Nc3ccc4oc5ccc6ccccc6c5c4c3)cccc21 ; CC1(C)c2ccccc2-c2c(Nc3cccc4c3-c3ccccc3C43c4ccccc4-c4ccccc43)cccc21]

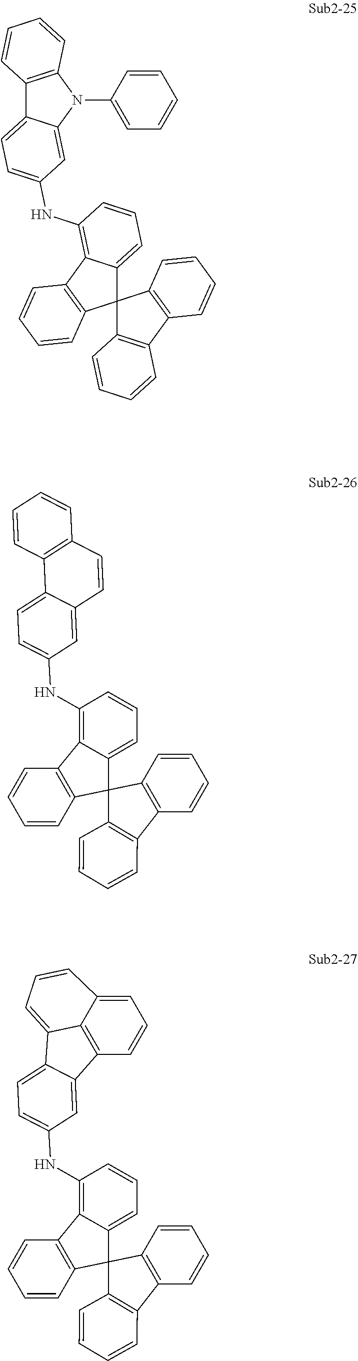 [SMILES: c1ccc(-n2c3ccccc3c3ccc(Nc4cccc5c4-c4ccccc4C54c5ccccc5-c5ccccc54)cc32)cc1 ; c1ccc2c(c1)-c1ccccc1C21c2ccccc2-c2c(Nc3ccc4c(ccc5ccccc54)c3)cccc21 ; c1ccc2c(c1)-c1ccccc1C21c2ccccc2-c2c(Nc3ccc4c(c3)-c3cccc5cccc-4c35)cccc21]

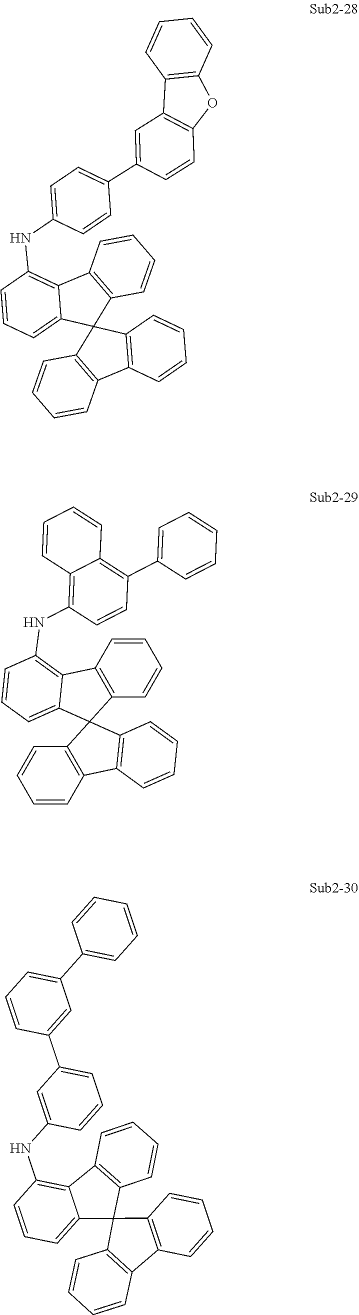 [SMILES: c1ccc2c(c1)-c1ccccc1C21c2ccccc2-c2c(Nc3ccc(-c4ccc5oc6ccccc6c5c4)cc3)cccc21 ; c1ccc(-c2ccc(Nc3cccc4c3-c3ccccc3C43c4ccccc4-c4ccccc43)c3ccccc23)cc1 ; c1ccc(-c2cccc(-c3cccc(Nc4cccc5c4-c4ccccc4C54c5ccccc5-c5ccccc54)c3)c2)cc1]

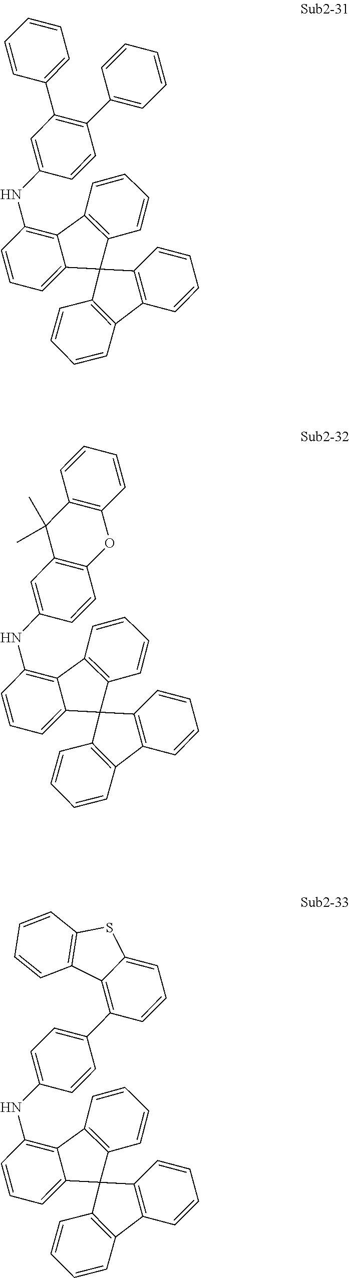 [SMILES: c1ccc(-c2ccc(Nc3cccc4c3-c3ccccc3C43c4ccccc4-c4ccccc43)cc2-c2ccccc2)cc1 ; CC1(C)c2ccccc2Oc2ccc(Nc3cccc4c3-c3ccccc3C43c4ccccc4-c4ccccc43)cc21 ; c1ccc2c(c1)-c1ccccc1C21c2ccccc2-c2c(Nc3ccc(-c4cccc5sc6ccccc6c45)cc3)cccc21]

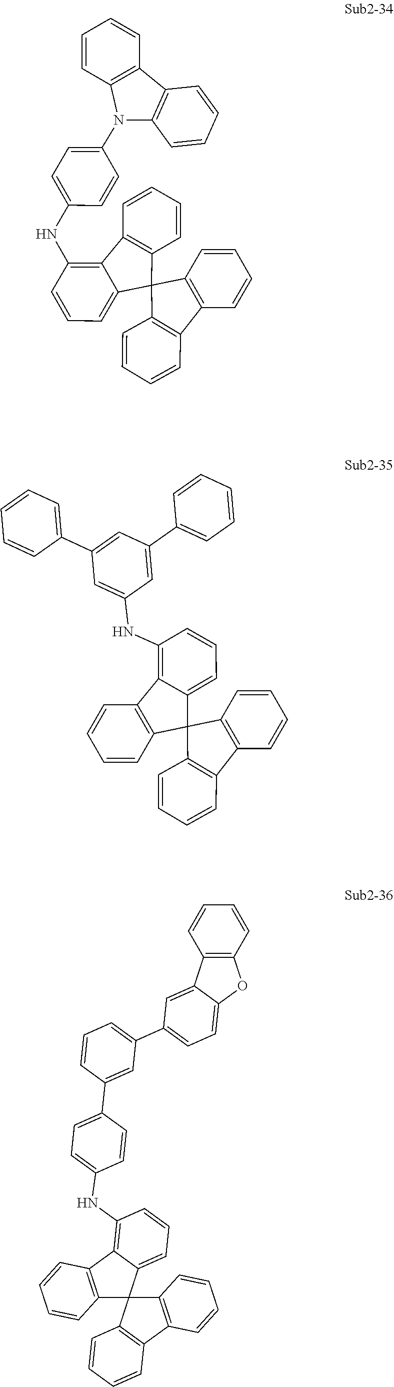 [SMILES: c1ccc2c(c1)-c1ccccc1C21c2ccccc2-c2c(Nc3ccc(-n4c5ccccc5c5ccccc54)cc3)cccc21 ; c1ccc(-c2cc(Nc3cccc4c3-c3ccccc3C43c4ccccc4-c4ccccc43)cc(-c3ccccc3)c2)cc1 ; c1cc(-c2ccc(Nc3cccc4c3-c3ccccc3C43c4ccccc4-c4ccccc43)cc2)cc(-c2ccc3oc4ccccc4c3c2)c1]

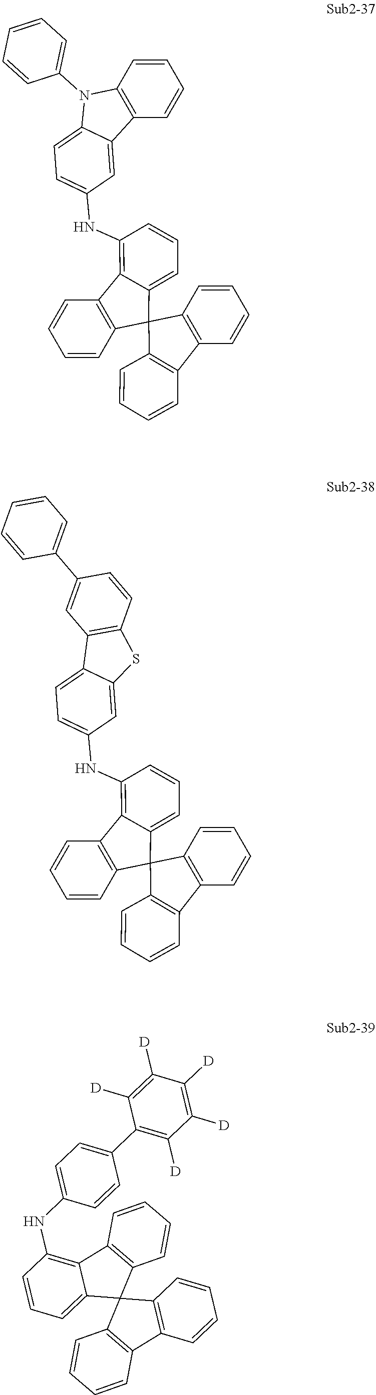 [SMILES: c1ccc(-n2c3ccccc3c3cc(Nc4cccc5c4-c4ccccc4C54c5ccccc5-c5ccccc54)ccc32)cc1 ; c1ccc(-c2ccc3sc4cc(Nc5cccc6c5-c5ccccc5C65c6ccccc6-c6ccccc65)ccc4c3c2)cc1 ; [2H]c1c([2H])c([2H])c(-c2ccc(Nc3cccc4c3-c3ccccc3C43c4ccccc4-c4ccccc43)cc2)c([2H])c1[2H]]

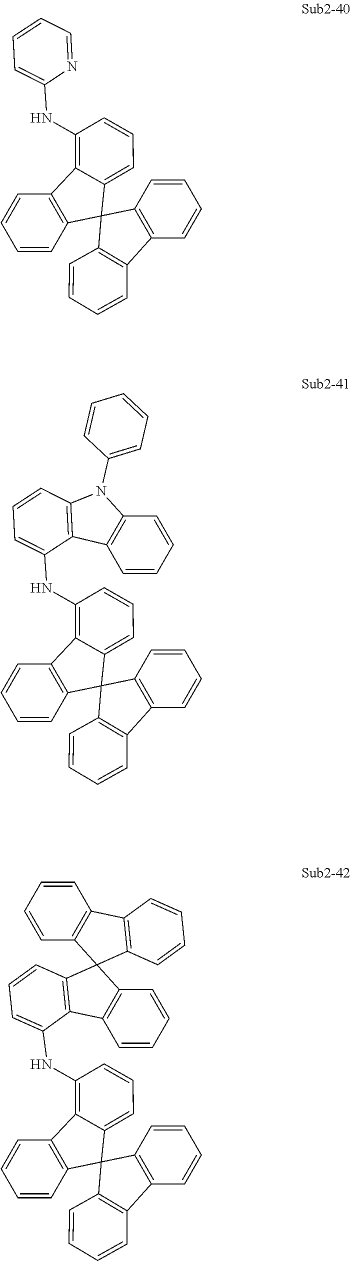 [SMILES: c1ccc(Nc2cccc3c2-c2ccccc2C32c3ccccc3-c3ccccc32)nc1 ; c1ccc(-n2c3ccccc3c3c(Nc4cccc5c4-c4ccccc4C54c5ccccc5-c5ccccc54)cccc32)cc1 ; c1ccc2c(c1)-c1ccccc1C21c2ccccc2-c2c(Nc3cccc4c3-c3ccccc3C43c4ccccc4-c4ccccc43)cccc21]

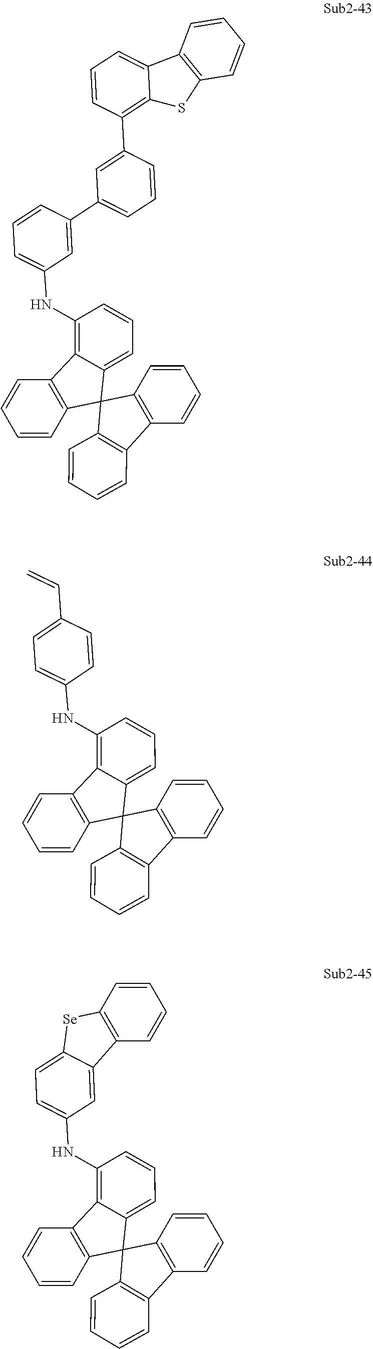 [SMILES: c1cc(Nc2cccc3c2-c2ccccc2C32c3ccccc3-c3ccccc32)cc(-c2cccc(-c3cccc4c3sc3ccccc34)c2)c1 ; C=Cc1ccc(Nc2cccc3c2-c2ccccc2C32c3ccccc3-c3ccccc32)cc1 ; c1ccc2c(c1)-c1ccccc1C21c2ccccc2-c2c(Nc3ccc4[se]c5ccccc5c4c3)cccc21]

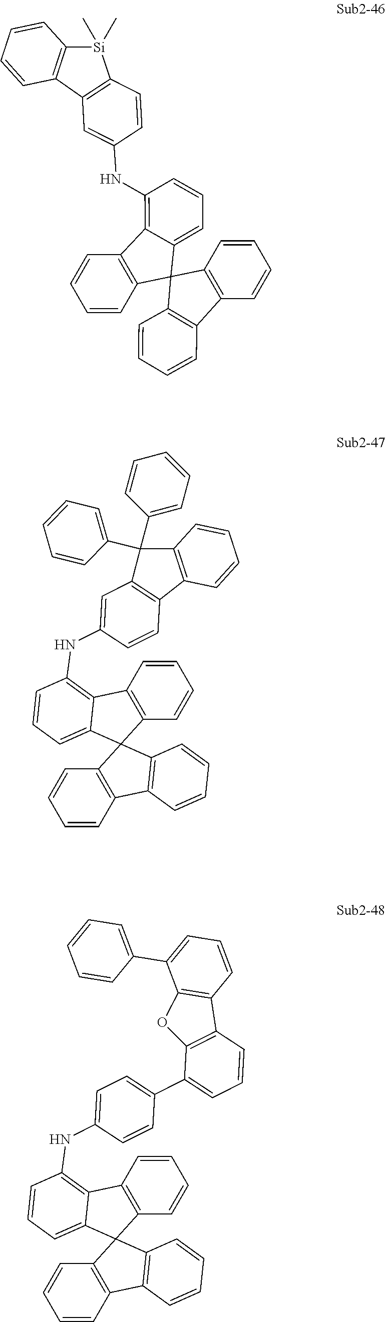 [SMILES: C[Si]1(C)c2ccccc2-c2cc(Nc3cccc4c3-c3ccccc3C43c4ccccc4-c4ccccc43)ccc21 ; c1ccc(C2(c3ccccc3)c3ccccc3-c3ccc(Nc4cccc5c4-c4ccccc4C54c5ccccc5-c5ccccc54)cc32)cc1 ; c1ccc(-c2cccc3c2oc2c(-c4ccc(Nc5cccc6c5-c5ccccc5C65c6ccccc6-c6ccccc65)cc4)cccc23)cc1]

TABLE 2

| Compound | FD-MS | Compound | FD-MS |
|---|---|---|---|
| Sub2-1 | m/z = 483.20(C₃₂H₂₅N = 483.61) | Sub2-4 | m/z = 497.18(C₃₂H₂₃NO = 497.60) |
| Sub2-6 | m/z = 523.23(C₄₀H₂₉N = 523.68) | Sub2-11 | m/z = 647.26(C₃₅H₂₃N = 457.58) |
| Sub2-13 | m/z = 589.19(C₄₃H₂₇NS = 589.76) | Sub2-15 | m/z = 411.03(C₅₀H₃₃N = 647.82) |
| Sub2-19 | m/z = 437.18(C₃₂H₂₃NO = 437.54) | Sub2-25 | m/z = 427.00(C₄₃H₂₈N₂ = 572.71) |
| Sub2-31 | m/z = 559.23(C₄₃H₂₉N = 559.71) | Sub2-40 | m/z = 408.16(C₃₀H₂₆N₂ = 408.50) |

II. Product Synthesis

After Sub 1 (1 eq) was dissolved in Toluene in a round-bottom flask, Sub 2 (1 eq), Pd₂(dba)₃ (0.03 eq), (t-Bu)3P (0.06 eq), and NaOt-Bu (3 eq) were added, followed by stirring at 100° C. Upon completion of the reaction, the reaction product was extracted with CH₂Cl₂ and water. The organic layer was dried over MgSO₄ and concentrated, and then the formed compound was subjected to silica gel column and recrystallization to give a final product.

1. Synthesis of P-1

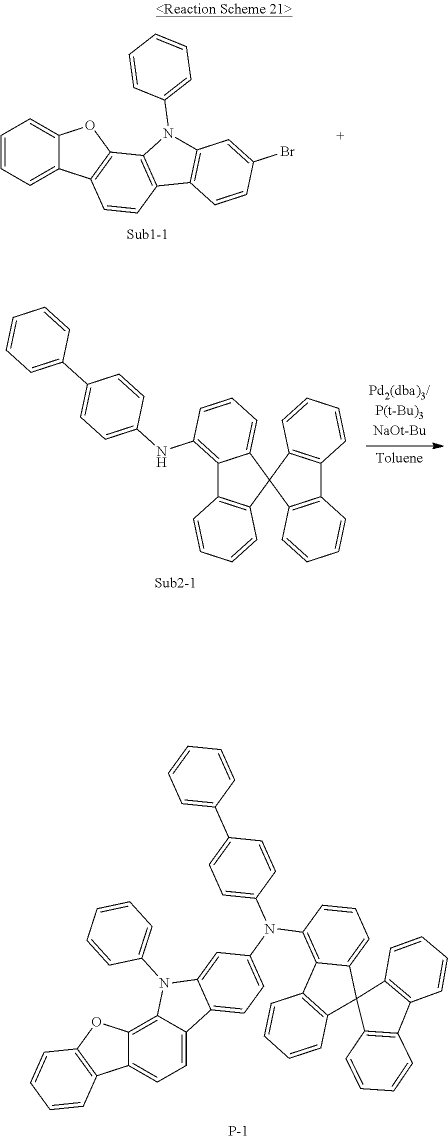

After Sub 1-1 (7.0 g, 17.0 mmol) obtained from the synthesis was dissolved in toluene (170 ml) in a round-bottom flask, Sub 2-1 (8.21 g, 17.0 mmol), Pd₂(dba)₃ (0.47 g, 0.5 mmol), 50% P(t-Bu)₃ (0.5 ml, 1.0 mmol), and NaOt-Bu (4.90 g, 50.9 mmol) were added, followed by stirring at 100° C. Upon completion of the reaction, the reaction product was extracted with CH₂Cl₂ and water. The organic layer was dried over MgSO₄ and concentrated, and then the formed compound was subjected to silica gel column and recrystallization to give a product 12.06 g (yield: 84%).

2. Synthesis of P-12

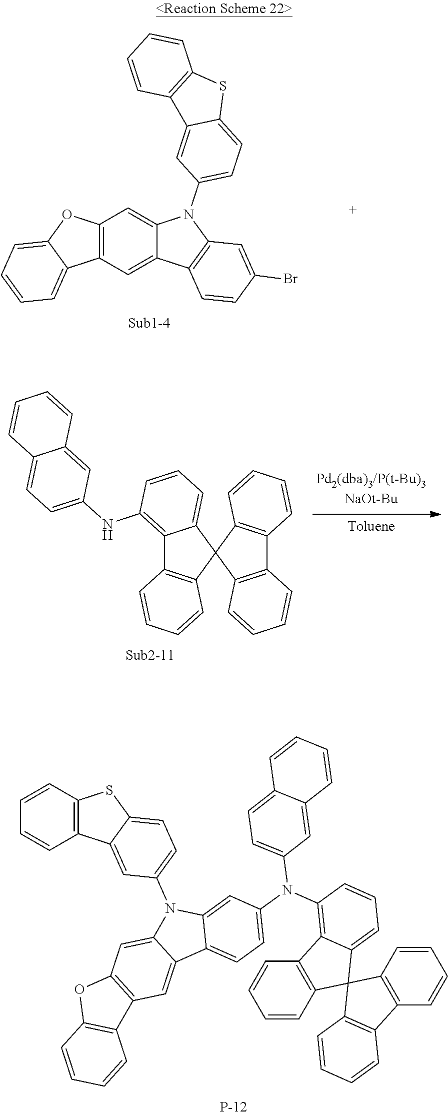

After Sub 1-7 (9.0 g, 17.4 mmol) obtained from the synthesis was dissolved in toluene (174 ml) in a round-bottom flask, Sub 2-11 (7.94 g, 17.4 mmol), Pd₂(dba)₃ (0.48 g, 0.5 mmol), 50% P(t-Bu)₃ (0.5 ml, 1.0 mmol), and NaOt-Bu (5.01 g, 52.1 mmol) were added, and subjected to the synthesis method of P-1 to give a product 12.74 g (yield: 82%).

3. Synthesis of P-21

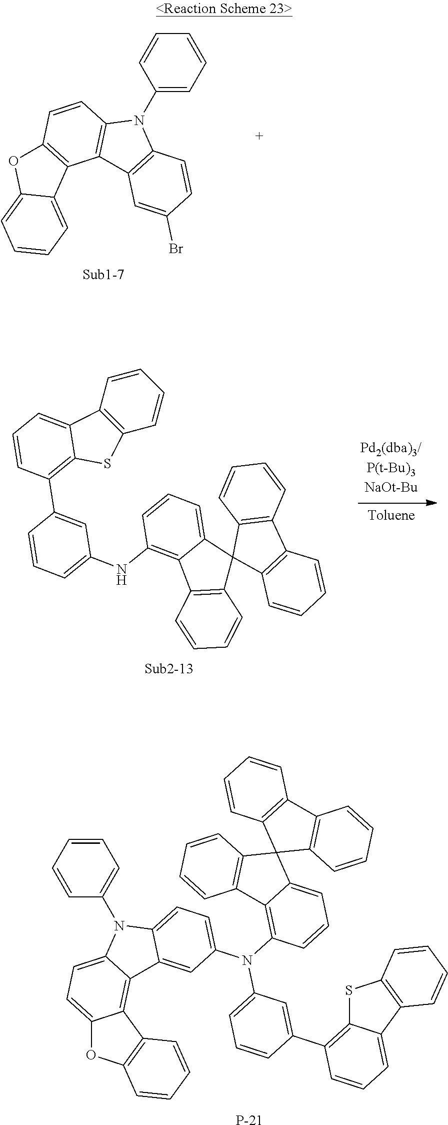

After Sub 1-7 (8.0 g, 19.4 mmol) obtained from the synthesis was dissolved in toluene (194 ml) in a round-bottom flask, Sub 2-13 (11.44 g, 19.4 mmol), Pd₂(dba)₃ (0.53 g, 0.6 mmol), 50% P(t-Bu)₃ (0.6 ml, 1.2 mmol), and NaOt-Bu (4.90 g, 50.9 mmol) were added, and subjected to the synthesis method of P-1 to give a product 12.06 g (yield: 84%).

4. Synthesis of P-24

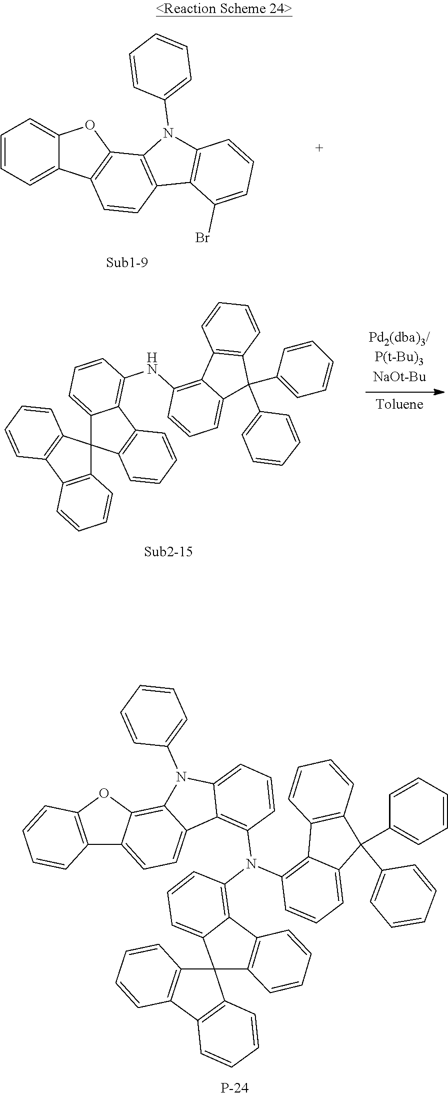

After Sub 1-9 (7.0 g, 17.0 mmol) obtained from the synthesis was dissolved in toluene (170 ml) in a round-bottom flask, Sub 2-15 (11.00 g, 17.0 mmol), Pd₂(dba)₃ (0.47 g, 0.5 mmol), 50% P(t-Bu)₃ (0.5 ml, 1.0 mmol), and NaOt-Bu (4.90 g, 50.9 mmol) were added, and subjected to the synthesis method of P-1 to give a product 13.47 g (yield: 81%).

5. Synthesis of P-28

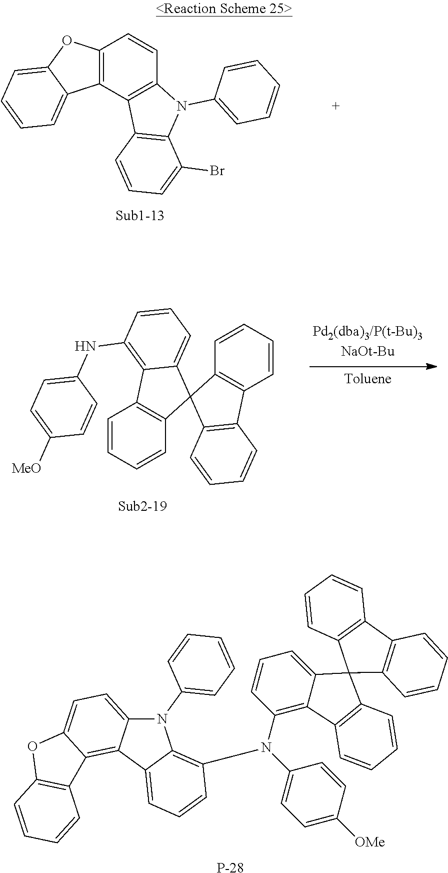

After Sub 1-13 (10.0 g, 24.3 mmol) obtained from the synthesis was dissolved in toluene (243 ml) in a round-bottom flask, Sub 2-19 (10.61 g, 24.3 mmol), Pd₂(dba)₃ (0.67 g, 0.7 mmol), 50% P(t-Bu)₃ (0.7 ml, 1.5 mmol), and NaOt-Bu (6.99 g, 72.8 mmol) were added, and subjected to the synthesis method of P-1 to give a product 10.82 g (yield: 58%).

6. Synthesis of P-41

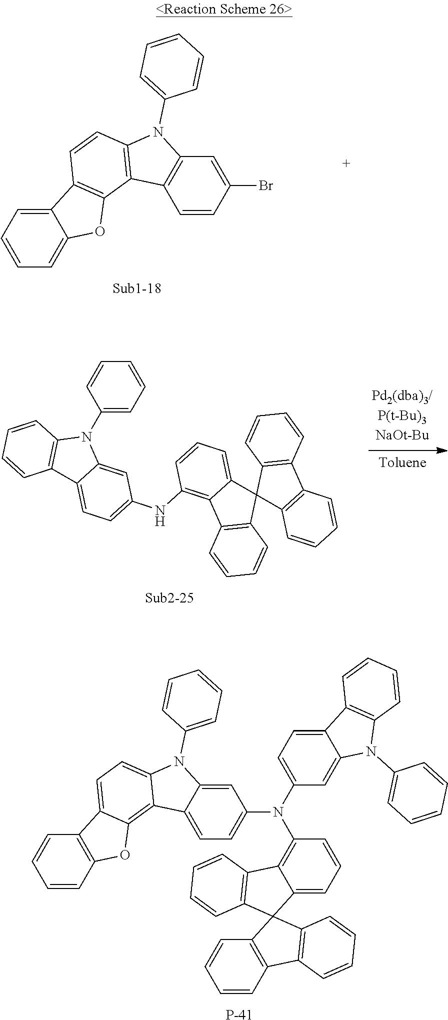

After Sub 1-18 (7.0 g, 17.0 mmol) obtained from the synthesis was dissolved in toluene (170 ml) in a round-bottom flask, Sub 2-25 (9.72 g, 17.0 mmol), Pd₂(dba)₃ (0.47 g, 0.5 mmol), 50% P(t-Bu)₃ (0.5 ml, 1.0 mmol), and NaOt-Bu (4.90 g, 50.9 mmol) were added, and subjected to the synthesis method of P-1 to give a product 10.44 g (yield: 68%).

7. Synthesis of P-55

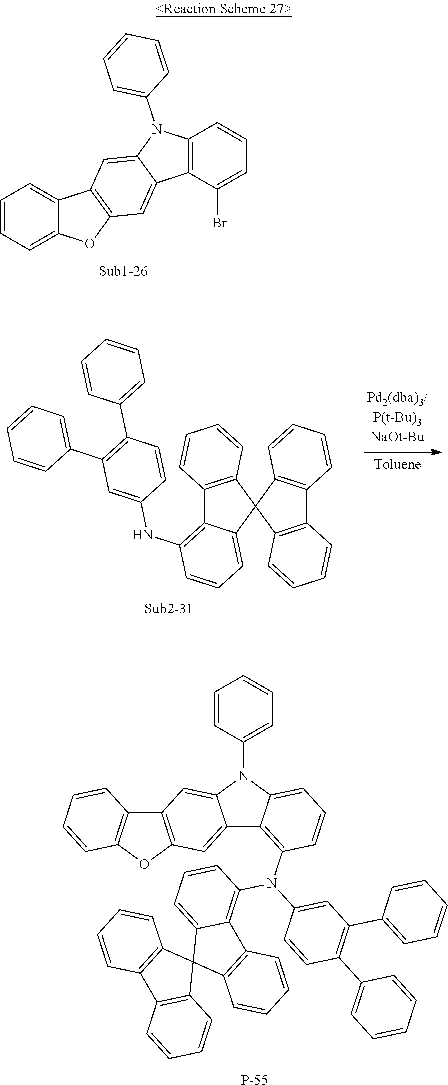

After Sub 1-1 (10.0 g, 24.3 mmol) obtained from the synthesis was dissolved in toluene (243 ml) in a round-bottom flask, Sub 2-31 (13.58 g, 24.3 mmol), Pd₂(dba)₃ (0.67 g, 0.7 mmol), 50% P(t-Bu)₃ (0.7 ml, 1.5 mmol), and NaOt-Bu (6.99 g, 72.8 mmol) were added, and subjected to the synthesis method of P-1 to give a product 14.05 g (yield: 65%).

8. Synthesis of P-61

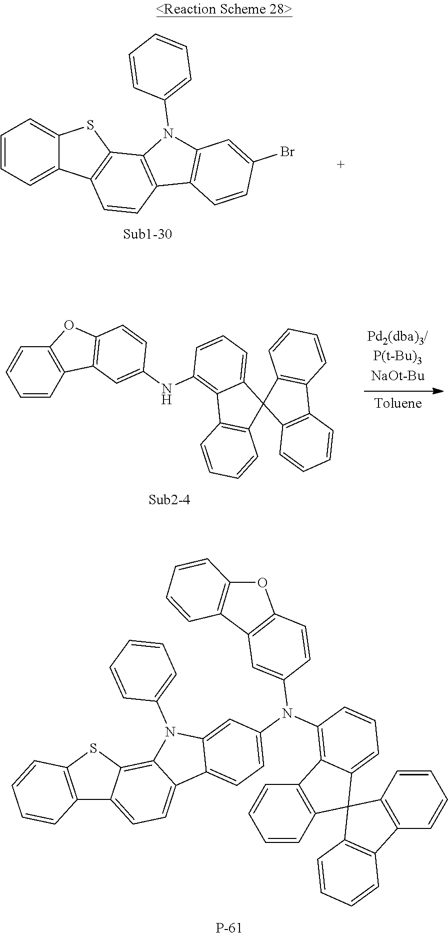

After Sub 1-30 (7.0 g, 16.3 mmol) obtained from the synthesis was dissolved in toluene (163 ml) in a round-bottom flask, Sub 2-4 (8.13 g, 16.3 mmol), Pd₂(dba)₃ (0.45 g, 0.5 mmol), 50% P(t-Bu)₃ (0.5 ml, 1.0 mmol), and NaOt-Bu (4.71 g, 49.0 mmol) were added, and subjected to the synthesis method of P-1 to give a product 10.50 g (yield: 76%).

9. Synthesis of P-72

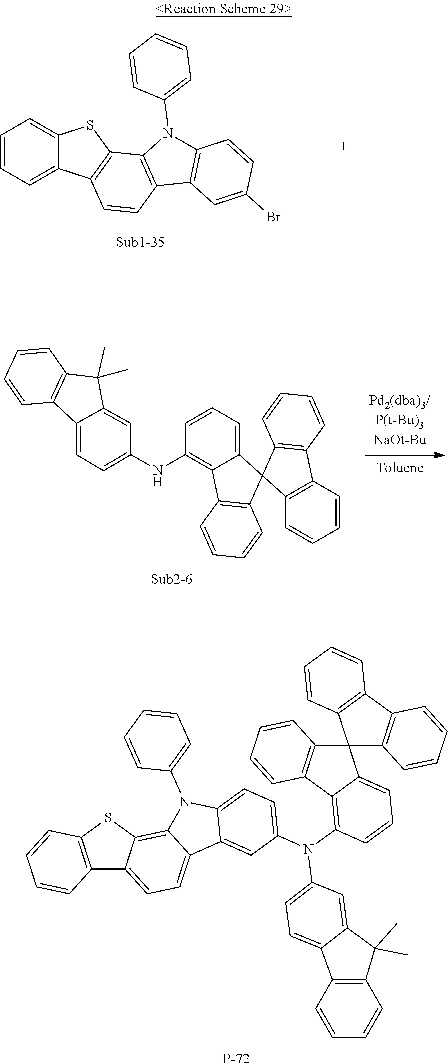

After Sub 1-35 (6.0 g, 14.0 mmol) obtained from the synthesis was dissolved in toluene (140 ml) in a round-bottom flask, Sub 2-6 (7.34 g, 14.0 mmol), Pd₂(dba)₃ (0.38 g, 0.4 mmol), 50% P(t-Bu)₃ (0.4 ml, 0.8 mmol), and NaOt-Bu (4.04 g, 42.0 mmol) were added, and subjected to the synthesis method of P-1 to give a product 9.76 g (yield: 80%).

10. Synthesis of P-80

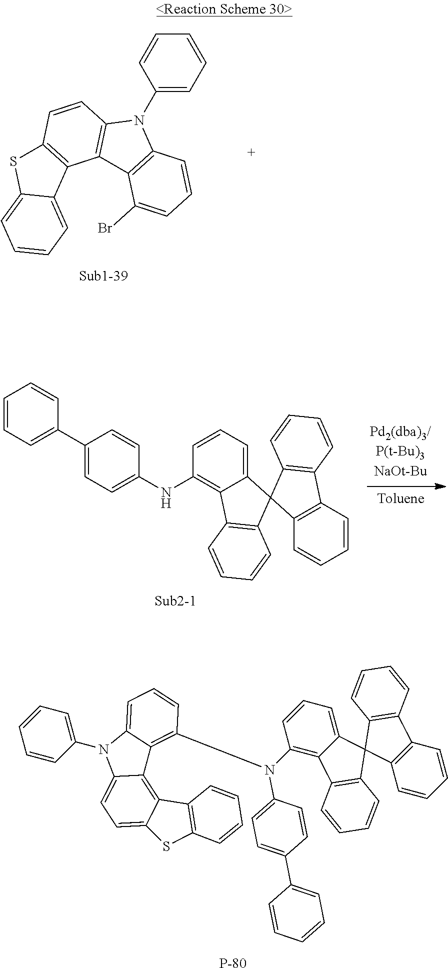

After Sub 1-39 (10.0 g, 23.3 mmol) obtained from the synthesis was dissolved in toluene (233 ml) in a round-bottom flask, Sub 2-1 (11.29 g, 23.3 mmol), Pd₂(dba)₃ (0.64 g, 0.7 mmol), 50% P(t-Bu)₃ (0.7 ml, 1.4 mmol), and NaOt-Bu (6.73 g, 70.0 mmol) were added, and subjected to the synthesis method of P-1 to give a product 12.03 g (yield: 62%).

11. Synthesis of P-87

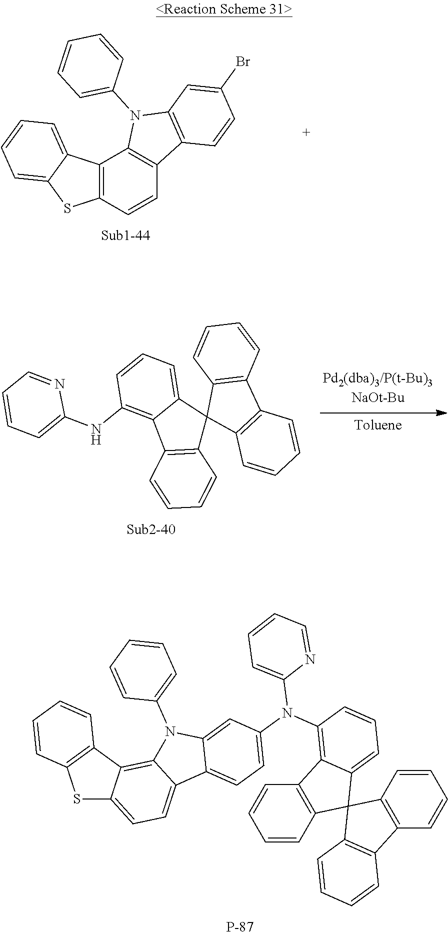

After Sub 1-44 (10.0 g, 23.3 mmol) obtained from the synthesis was dissolved in toluene (233 ml) in a round-bottom flask, Sub 2-40 (9.54 g, 23.3 mmol), Pd₂(dba)₃ (0.64 g, 0.7 mmol), 50% P(t-Bu)₃ (0.7 ml, 1.4 mmol), and NaOt-Bu (6.73 g, 70.0 mmol) were added, and subjected to the synthesis method of P-1 to give a product 12.00 g (yield: 68%).

12. Synthesis of P-98

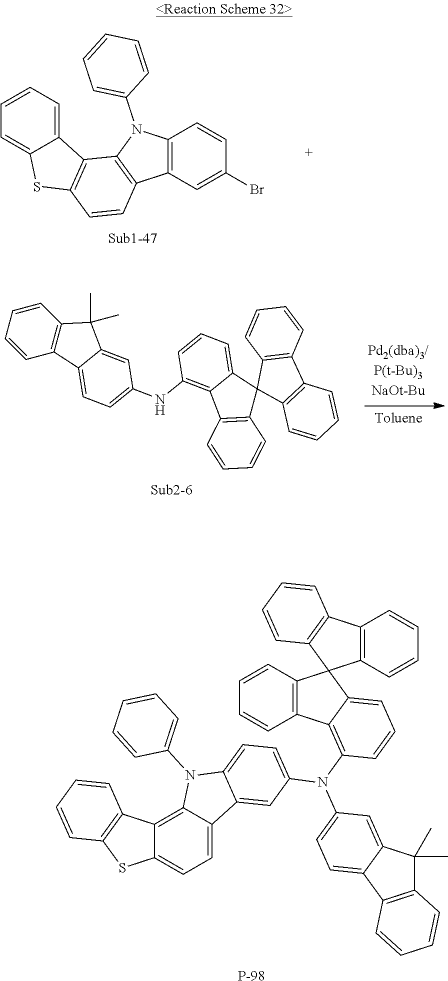

After Sub 1-47 (7.0 g, 16.3 mmol) obtained from the synthesis was dissolved in toluene (163 ml) in a round-bottom flask, Sub 2-6 (8.56 g, 16.3 mmol), Pd₂(dba)₃ (0.45 g, 0.5 mmol), 50% P(t-Bu)₃ (0.5 ml, 1.0 mmol), and NaOt-Bu (4.71 g, 49.0 mmol) were added, and subjected to the synthesis method of P-1 to give a product 11.25 g (yield: 79%).

TABLE 3

| Compound | FD-MS | Compound | FD-MS |
|---|---|---|---|
| P-1 | m/z = 814.30(C₈₁H₃₃N₂O = 814.99) | P-2 | m/z = 814.30(C₆₁H₃₈N₂ O = 814.99) |
| P-3 | m/z = 890.33(C₆₇H₄₂N₂O = 891.09) | P-4 | m/z = 828.28(C₆₁H₃₆N₂O₂ = 828.97) |
| P-5 | m/z = 844.25(C₈₁H₃₆N₂OS = 845.03) | P-6 | m/z = 854.33(C₆₁H₄₂N₂O = 855.05) |
| P-7 | m/z = 978.36(C₇₄H₄₆N₂O = 979.20) | P-8 | m/z = 904.31(C₆₇H₄₀N₂O₂ = 905.07) |
| P-9 | m/z = 894.27(C₈₅H₃₈N₂OS = 895.09) | P-10 | m/z = 904.31(C₆₇H₄₀N₂O₂ = 905.07) |
| P-11 | m/z = 814.30(C₆₁H₃₈N₂O = 814.99) | P-12 | m/z = 894.27(C₆₅H₃₈N₂OS = 895.09) |
| P-13 | m/z = 814.30(C₆₁H₃₈N₂O = 814.99) | P-14 | m/z = 890.33(C₆₇H₄₂N₂O = 891.09) |
| P-15 | m/z = 920.29(C₆₇H₄₀N₂OS = 921.13) | P-16 | m/z = 814.30(C₆₁H₃₈N₂O = 814.99) |
| P-17 | m/z = 814.30(C₆₁H₃₈N₂O = 814.99) | P-18 | m/z = 814.30(C₆₁H₃₈N₂O = 814.99) |
| P-19 | m/z = 828.28(C₆₁H₃₆N₂O₂ = 828.97) | P-20 | m/z = 814.30(C₆₁H₃₈N₂O = 814.99) |
| P-21 | m/z = 920.29(C₆₇H₄₀N₂OS = 921.13) | P-22 | m/z = 814.30(C₆₁H₃₈N₂O = 814.99) |
| P-23 | m/z = 842.33(C₆₃H₄₂N₂O = 843.04) | P-24 | m/z = 978.36(C₇₄H₄₆N₂O = 979.20) |
| P-25 | m/z = 763.26(C₅₆H₃₃N₃O = 763.90) | P-26 | m/z = 828.28(C₆₁H₃₆N₂O₂ = 828.97) |
| P-27 | m/z = 815.29(C₆₀H₃₇N₃O = 815.98) | P-28 | m/z = 768.28(C₅₆H₃₆N₂O₂ = 768.92) |
| P-29 | m/z = 814.30(C₆₁H₃₈N₂O = 814.99) | P-30 | m/z = 814.30(C₆₁H₃₈N₂O₂ = 814.99) |
| P-31 | m/z = 854.33(C₆₄H₄₂N₂O = 855.05) | P-32 | m/z = 844.25(C₆₁H₃₈N₂OS = 845.03) |
| P-33 | m/z = 828.28(C₆₁H₃₆N₂O₂ = 828.97) | P-34 | m/z = 940.35(C₇₁H₄₄N₂O = 914.15) |
| P-35 | m/z = 966.36(C₇₃H₄₆N₂O = 967.18) | P-36 | m/z = 854.33(C₆₄H₄₂N₂O = 855.05) |
| P-37 | m/z = 814.30(C₆₁H₃₀N₂O = 814.99) | P-38 | m/z = 814.30(C₆₁H₃₈N₂O = 814.99) |
| P-39 | m/z = 878.29(C₆₅H₃₈N₂O₂ = 879.03) | P-40 | m/z = 854.33(C₆₄H₄₂N₂O = 855.05) |
| P-41 | m/z = 903.32(C₆₇H₄₁N₂O = 904.09) | P-42 | m/z = 814.30(C₆₁H₃₈N₂O = 814.99) |
| P-43 | m/z = 838.30(C₆₃H₃₈N₂O = 839.01) | P-44 | m/z = 880.29(C₆₅H₃₇FN₂O = 881.02) |
| P-45 | m/z = 814.30(C₆₁H₃₈N₂O = 814.99) | P-46 | m/z = 854.33(C₆₄H₄₂N₂O = 855.05) |
| P-47 | m/z = 828.28(C₆₁H₃₈N₂O₂ = 828.97) | P-48 | m/z = 814.30(C₆₁H₃₈N₂O = 814.99) |
| P-49 | m/z = 854.33(C₆₄H₄₂N₂O = 855.05) | P-50 | m/z = 814.30(C₆₁H₃₈N₂O = 814.99) |
| P-51 | m/z = 814.30(C₆₁H₃₈N₂O = 814.99) | P-52 | m/z = 904.31(C₆₇H₄₀N₂O₂ = 905.07) |
| P-53 | m/z = 864.31(C₆₅H₄₈N₂O = 865.05) | P-54 | m/z = 890.33(C₆₇H₄₂N₂O = 891.09) |
| P-55 | m/z = 890.33(C₆₇H₄₂N₂O = 891.09) | P-56 | m/z = 870.32(C₆₄H₄₂N₂O₂ = 871.05) |
| P-57 | m/z = 920.29(C₆₇H₄₀N₂OS = 921.13) | P-58 | m/z = 903.32(C₆₇H₄₁N₃O = 904.09) |
| P-59 | m/z = 830.28(C₆₁H₃₈N₂S = 831.05) | P-60 | m/z = 870.31(C₆₄H₄₂N₂S = 871.11) |
| P-61 | m/z = 844.25(C₆₁H₃₆N₂OS = 845.03) | P-62 | m/z = 936.26(C₆₁H₄₀N₂S₂ = 937.19) |
| P-63 | m/z = 906.31(C₆₇H₄₂N₂S = 907.15) | P-64 | m/z = 996.32(C₇₂H₄₄N₂OS = 997.23) |
| P-65 | m/z = 919.30(C₆₇H₄₁N₂S = 920.15) | P-66 | m/z = 936.26(C₆₇H₄₀N₂S₂ = 937.19) |
| P-67 | m/z = 830.28(C₆₁H₃₈N₂S = 831.05) | P-68 | m/z = 854.28(C₆₈H₃₈N₂S = 855.07) |
| P-69 | m/z = 830.28(C₆₁H₃₈N₂S = 831.05) | P-70 | m/z = 810.31(C₁₄H₄₂N₂S = 811.06) |
| P-71 | m/z = 830.28(C₆₁H₃₈N₂S = 831.05) | P-72 | m/z = 870.31(C₆₄H₄₂N₂S = 871.11) |
| P-73 | m/z = 844.25(C₆₁H₃₈N₂OS = 845.03) | P-74 | m/z = 830.28(C₆₁H₃₈N₂S = 831.05) |
| P-75 | m/z = 835.31(C₆₁H₃₃D₃N₂S = 836.08) | P-76 | m/z = 860.23(C₆₁H₃₆N₂S₂ = 861.09) |
| P-77 | m/z = 830.28(C₆₁H₃₈N₂S = 831.05) | P-78 | m/z = 870.31(C₆₄H₄₂N₂S = 871.11) |
| P-79 | m/z = 830.28(C₆₁H₃₈N₂S = 831.05) | P-80 | m/z = 830.28(C₆₁H₃₆N₂S = 831.05) |
| P-81 | m/z = 830.28(C₆₁H₃₈N₂S = 831.05) | P-82 | m/z = 830.28(C₆₁H₃₈N₂S = 831.05) |
| P-83 | m/z = 830.28(C₆₁H₃₈N₂S = 831.05) | P-84 | m/z = 830.28(C₆₁H₃₈N₂S = 831.05) |
| P-85 | m/z = 830.28(C₆₁H₃₈N₂S = 831.05) | P-86 | m/z = 870.31(C₆₄H₄₂N₂S = 871.11) |
| P-87 | m/z = 755.24(C₅₄H₃₃N₂S = 755.94) | P-88 | m/z = 919.30(C₆₁H₄₁N₃S = 920.15) |
| P-89 | m/z = 992.32(C₇₄H₄₄N₂S = 993.24) | P-90 | m/z = 1012.29(C₇₃H₄₄N₂S₂ = 1031.29) |
| P-91 | m/z = 830.28(C₆₁H₃₈N₂S = 831.05) | P-92 | m/z = 780.26(C₆₇H₃₆N₂S = 780.99) |
| P-93 | m/z = 830.28(C₆₁H₃₈N₂S = 831.05) | P-94 | m/z = 870.31(C₆₄H₄₂N₂S = 871.11) |
| P-95 | m/z = 908.18(C₆₁H₃₈N₂SSe = 907.99) | P-96 | m/z = 886.28(C₆₁H₄₂N₂SSi = 887.19) |
| P-97 | m/z = 830.28(C₆₁H₃₈N₂S = 831.05) | P-98 | m/z = 870.31(C₆₄H₄₂N₂S = 871.11) |
| P-99 | m/z = 993.34(C₇₄H₄₆N₂S = 995.26) | P-100 | m/z = 996.32(C₇₃H₄₄N₂OS = 997.23) |
| P-101 | m/z = 830.28(C₆₁H₃₈N₂S = 831.05) | P-102 | m/z = 880.29(C₆₆H₄₀N₂S = 881.11) |
| P-103 | m/z = 830.28(C₆₁H₃₈N₂S = 831.05) | P-104 | m/z = 870.31(C₆₄H₄₂N₂S = 871.11) |
| P-105 | m/z = 830.28(C₆₁H₃₈N₂S = 831.05) | P-106 | m/z = 830.28(C₆₁H₃₈N₂S = 831.05) |
| P-107 | m/z = 830.28(C₆₁H₃₈N₂S = 831.05) | P-108 | m/z = 830.28(C₆₁H₃₈N₂S = 831.05) |
| P-109 | m/z = 830.28(C₆₁H₃₈N₂S = 831.05) | P-110 | m/z = 830.28(C₆₁H₃₈N₂S = 831.05) |

Although the exemplary synthesis examples of the present disclosure represented by Formula 1 have been described above, the synthesis examples are on the basis of Buchwald-Hartwig cross coupling, Suzuki cross-coupling, intramolecular acid-induced cyclization (J. mater. Chem. 1999, 9, 2095.), Pd(II)-catalyzed oxidative cyclization (Org. Lett. 2011, 13, 5504), Grignard reaction, Cyclic Dehydration, and PPh₃-mediated reductive cyclization (J. Org. Chem. 2005, 70, 5014.). A person skilled in the art could easily understand that the above reactions proceed even though, besides the substituents specified in the specific synthesis examples, other substituents defined in Formula 1 are bound.

Manufacturing and Evaluation of Organic Electric Elements

### [Example 1] Green Organic Light Emitting Diode

### (Light Emitting Auxiliary Layer)

An organic light emitting diode was manufactured by an ordinary method using the compound of the present disclosure as a material for a hole transport layer. First, a film of N¹-(naphthalen-2-yl)-N⁴,N⁴-bis(4-(naphthalen-2-yl(phenyl)amino)phenyl)-N¹-phenylbenzene-1,4-diamine (hereinafter, abbreviated as “2-TNATA”) as a hole injection layer was formed with a thickness of 60 nm through vacuum deposition on an ITO layer (anode) formed on a glass substrate. Then, on this film, 4,4-bis[N-(1-naphthyl)-N-phenylamino]biphenyl (hereinafter, abbreviated as “-NPD”) as a hole transport compound was vacuum-deposited with a thickness of 60 nm to form a hole transport layer. Subsequently, Compound P-37 as a material for a light emitting auxiliary was vacuum-deposited with a thickness of 20 nm to form a light emitting auxiliary layer. After the light emitting auxiliary layer was formed, a light emitting layer with a thickness of 30 nm was deposited on the light emitting auxiliary layer by doping an upper portion of the light emitting auxiliary layer with 4,4′-N,N′-dicarbazole-biphenyl (CBP) as a host and tris(2-phenylpyridine)-iridium (Ir(ppy)₃) as a dopant at a weight ratio of 95:5. Then, (1,1′-bisphenyl)-4-olato)bis(2-methyl-8-quinolinolato)aluminum (hereinafter abbreviated as “BAlq”) was vacuum-deposited with a thickness of 10 nm for a hole blocking layer, and tris(8-quinolinol)aluminum (hereinafter abbreviated as “Alq₃”) was formed with a thickness of 40 nm for an electron injection layer. Thereafter, LiF, which is a halogenated alkali metal, was deposited with a thickness of 0.2 nm for an electron injection layer, and subsequently Al was deposited with a thickness of 150 nm and used as a cathode. In this way, the organic light emitting diode was manufactured.

A forward bias DC voltage was applied to each of the organic light emitting diodes manufactured in examples and comparative examples to measure electro-luminescent (EL) characteristics thereof by PR-650 (Photoresearch). As a result, the T95 lifetime was measured by a lifetime measurement equipment (fabricated by Mcscience) at reference brightness of 5000 cd/m². The element manufacturing and evaluation results are shown in the table below.

### [Example 2] to [Example 45] Green Organic Light Emitting Diodes

Organic light emitting diodes were manufactured by the same method as in Example 1 except that as a material for the light emitting auxiliary layer, the compounds of the present disclosure shown in table 4 below were used instead of Compound P-37 according to example 1 of the present disclosure.

### Comparative Example 1

An organic light emitting diode was manufactured by the same method as in Example 1 except that the light emitting auxiliary layer is not used.

### [Comparative Example 2] to [Comparative Example 3]

Organic light emitting diodes were manufactured by the same method as in Example 1 except that as a material for the light emitting auxiliary layer, any one of Comparative compounds 1 to 2 shown in Table 4 below was used instead of Compound P-37 according to example 1 of the present disclosure.

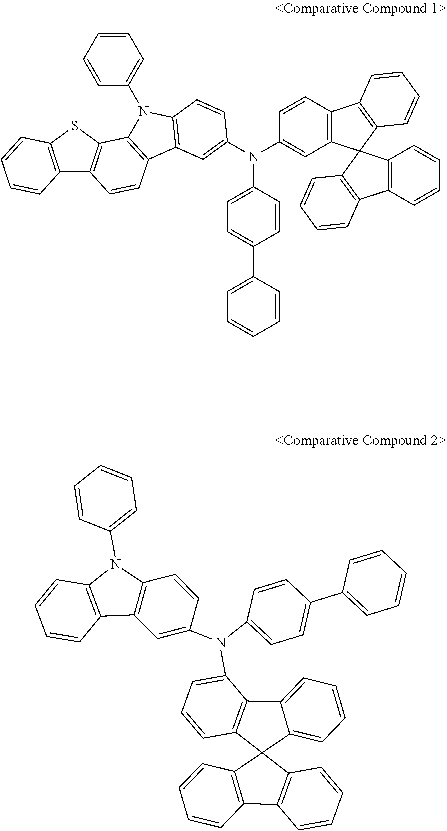 [SMILES: c1ccc(-c2ccc(N(c3ccc4c(c3)C3(c5ccccc5-c5ccccc53)c3ccccc3-4)c3ccc4c(c3)c3ccc5c6ccccc6sc5c3n4-c3ccccc3)cc2)cc1 ; c1ccc(-c2ccc(N(c3ccc4c(c3)c3ccccc3n4-c3ccccc3)c3cccc4c3-c3ccccc3C43c4ccccc4-c4ccccc43)cc2)cc1]

TABLE 4

| | | Driving | Current | Brightness | Efficiency | | CIE | |
|---|---|---|---|---|---|---|---|---|
| | Compound | voltage | (mA/cm²) | (cd/m²) | (cd/A) | T(95) | X | Y |
| Comparative | No light emitting | 6 | 21.6 | 5000 | 23.2 | 57.4 | 0.33 | 0.62 |
| Example (1) | auxiliary layer | | | | | | | |
| Comparative | Comparative | 5.8 | 12.8 | 5000 | 39.1 | 113.2 | 0.32 | 0.62 |
| Example (2) | Compound 1 | | | | | | | |
| Comparative | Comparative | 5.7 | 11.7 | 5000 | 42.7 | 101.6 | 0.32 | 0.61 |
| Example (3) | Compound 2 | | | | | | | |
| Example (1) | Compound P-37 | 4.9 | 7.3 | 5000 | 68.8 | 150.0 | 0.33 | 0.61 |
| Example (2) | Compound P-39 | 4.9 | 7.2 | 5000 | 69.7 | 154.5 | 0.32 | 0.61 |
| Example (3) | Compound P-40 | 4.9 | 7.3 | 5000 | 68.7 | 150.7 | 0.32 | 0.62 |
| Example (4) | Compound P-41 | 4.9 | 7.3 | 5000 | 68.3 | 150.9 | 0.32 | 0.62 |
| Example (5) | Compound P-33 | 5.1 | 7.8 | 5000 | 64.1 | 144.9 | 0.32 | 0.61 |
| Example (6) | Compound P-34 | 5.1 | 7.9 | 5000 | 63.3 | 143.8 | 0.33 | 0.62 |
| Example (7) | Compound P-36 | 5.1 | 7.9 | 5000 | 63.3 | 143.2 | 0.33 | 0.61 |
| Example (8) | Compound P-9 | 5.2 | 8.4 | 5000 | 59.2 | 136.9 | 0.33 | 0.61 |
| Example (9) | Compound P-10 | 5.2 | 8.7 | 5000 | 57.4 | 137.2 | 0.32 | 0.61 |
| Example (10) | Compound P-12 | 5.3 | 8.6 | 5000 | 57.9 | 132.7 | 0.33 | 0.62 |
| Example (11) | Compound P-43 | 5.4 | 9.0 | 5000 | 55.5 | 130.7 | 0.32 | 0.61 |
| Example (12) | Compound P-13 | 5.4 | 9.0 | 5000 | 55.4 | 131.4 | 0.32 | 0.61 |
| Example (13) | Compound P-49 | 5.0 | 7.5 | 5000 | 66.9 | 146.4 | 0.33 | 0.62 |
| Example (14) | Compound P-48 | 5.0 | 7.6 | 5000 | 66.1 | 146.5 | 0.32 | 0.61 |
| Example (15) | Compound P-45 | 5.2 | 8.0 | 5000 | 62.5 | 139.5 | 0.32 | 0.62 |
| Example (16) | Compound P-47 | 5.2 | 8.2 | 5000 | 61.1 | 139.2 | 0.33 | 0.62 |
| Example (17) | Compound P-18 | 5.3 | 8.5 | 5000 | 58.5 | 133.6 | 0.33 | 0.62 |
| Example (18) | Compound P-23 | 5.3 | 8.7 | 5000 | 57.5 | 133.1 | 0.33 | 0.61 |
| Example (19) | Compound P-52 | 5.3 | 8.9 | 5000 | 56.4 | 134.5 | 0.33 | 0.61 |
| Example (20) | Compound P-21 | 5.3 | 8.7 | 5000 | 57.3 | 134.9 | 0.33 | 0.61 |
| Example (21) | Compound P-24 | 5.5 | 9.2 | 5000 | 54.2 | 124.9 | 0.33 | 0.61 |
| Example (22) | Compound P-27 | 5.5 | 9.2 | 5000 | 54.3 | 125.8 | 0.33 | 0.61 |
| Example (23) | Compound P-57 | 5.5 | 9.5 | 5000 | 52.4 | 123.2 | 0.33 | 0.61 |
| Example (24) | Compound P-93 | 5.1 | 8.1 | 5000 | 62.1 | 142.8 | 0.32 | 0.62 |
| Example (25) | Compound P-94 | 5.1 | 8.0 | 5000 | 62.3 | 142.3 | 0.33 | 0.62 |
| Example (26) | Compound P-96 | 5.1 | 8.0 | 5000 | 62.7 | 140.6 | 0.33 | 0.62 |
| Example (27) | Compound P-89 | 5.2 | 8.7 | 5000 | 57.8 | 138.6 | 0.32 | 0.62 |
| Example (28) | Compound P-90 | 5.2 | 8.4 | 5000 | 59.5 | 138.6 | 0.32 | 0.61 |
| Example (29) | Compound P-61 | 5.4 | 8.9 | 5000 | 55.9 | 129.9 | 0.32 | 0.61 |
| Example (30) | Compound P-69 | 5.4 | 9.1 | 5000 | 54.7 | 129.3 | 0.32 | 0.62 |
| Example (31) | Compound P-91 | 5.4 | 9.0 | 5000 | 55.4 | 129.8 | 0.32 | 0.61 |
| Example (32) | Compound P-15 | 5.4 | 9.1 | 5000 | 54.7 | 130.1 | 0.32 | 0.61 |
| Example (33) | Compound P-103 | 5.2 | 8.8 | 5000 | 57.0 | 137.5 | 0.33 | 0.62 |
| Example (34) | Compound P-104 | 5.2 | 8.8 | 5000 | 57.1 | 136.2 | 0.33 | 0.61 |
| Example (35) | Compound P-97 | 5.3 | 8.8 | 5000 | 56.9 | 133.0 | 0.33 | 0.62 |
| Example (36) | Compound P-98 | 5.3 | 8.8 | 5000 | 57.0 | 133.4 | 0.32 | 0.62 |
| Example (37) | Compound P-100 | 5.3 | 8.8 | 5000 | 57.0 | 132.9 | 0.32 | 0.62 |
| Example (38) | Compound P-72 | 5.4 | 9.3 | 5000 | 53.5 | 123.7 | 0.33 | 0.61 |
| Example (39) | Compound P-77 | 5.4 | 9.4 | 5000 | 53.3 | 125.7 | 0.32 | 0.61 |
| Example (40) | Compound P-102 | 5.4 | 9.3 | 5000 | 53.6 | 123.6 | 0.33 | 0.62 |
| Example (41) | Compound P-75 | 5.4 | 9.5 | 5000 | 52.9 | 125.8 | 0.33 | 0.62 |
| Example (42) | Compound P-106 | 5.5 | 9.1 | 5000 | 54.9 | 128.6 | 0.32 | 0.61 |
| Example (43) | Compound P-110 | 5.5 | 9.6 | 5000 | 52.3 | 124.4 | 0.33 | 0.62 |
| Example (44) | Compound P-111 | 5.6 | 10.0 | 5000 | 50.0 | 123.9 | 0.33 | 0.61 |
| Example (45) | Compound P-112 | 5.6 | 10.1 | 5000 | 49.7 | 121.1 | 0.32 | 0.62 |

As can be seen from the results of Table 4, when the green organic light emitting diodes were manufactured using the materials for an organic light emitting diode of the present disclosure as a material for the light emitting auxiliary layer, the driving voltage of the organic light emitting diodes can be lowered and the light emission efficiency and lifetime can be remarkably improved, as compared with when the light emitting auxiliary layer was not used or Comparative Examples 2 and 3 using Comparative Compounds 1 and 2.

In other words, Comparative Example 1 using no light emitting auxiliary layer showed the worst results; out of the comparative examples, Comparative Example 2 using Comparative Compound 1 in which the 5-membered ring and 2-spirofluorene are substituted showed better results. Also, compounds in which carbazole, which is a general hetero-ring, and 4-spirofluorene are substituted showed much better results, and the present inventive compounds in which 4-spirofluorene and the 5-membered ring are substituted showed the best results.

Meanwhile, two types of trends can be seen from the data of the elements.

The first is a difference according to the substituent position (4-position vs. 2-position) of spirofluorene, which can be seen from the comparison between Comparative Example 2 and Examples 1 to 45 using the inventive compounds.

Compared with Comparative Compound 1 with a substitution of 2-spirofluorene, Comparative Compound 2 with a substitution of 4-spirofluorene showed better results, and the reason is considered to be that the compound with a substitution of spirofluorene at the 4-position had a deeper HOMO level than the compound with a substitution of spirofluorene at the 2-position. As the HOMO level is deeper, more holes in the light emitting layer move fast and easily, leading to an increase in charge balance of holes and electrons in the light emitting layer, so that light emission well occurs inside the light emitting layer but not in the interface of the hole transport layer, and as a result, the deterioration is also reduced in the ITL and HTL interfaces, thereby maximizing a driving voltage, efficiency, and lifetime of the element. Therefore, the advantage of 4-spirofluorene can be confirmed from the results.

The second is a difference in view of carbazole and the 5-membered ring, which can be seen from the comparison between Comparative Compound 2 and the present inventive compounds. Compared with Comparative Compound 2 in which carbazole is substituted to 4-spirofluorene, the present inventive compounds in which the 5-membered ring is substituted to 4-spriofluorenee resulted in excellent efficiency. The reason is considered to be that there are more spaces capable of trapping holes when the 5-membered ring rather than carbazole is substituted to the tertiary amine including 4-spriofluorene, and as a result, the charge balance in the light emitting layer is more excellent, leading to an increase in efficiency.

Therefore, it can be confirmed that the present inventive compounds in which the 5-membered ring is substituted to 4-spirofluorene showed significantly excellent performance compared with existing similar compounds.

As described in the above results, it can be confirmed that the properties of compounds are varied according to the kinds and positions of substituents, which function as important factors in the improvement in element performance, resulting in different results. That is, it is suggested that properties of compounds and results of elements are significantly varied as 4-spirofluorene and a 5-membered ring are substituted to a tertiary amine.

### Example 46

The indium tin oxide (ITO) layer was patterned to have a light emitting area of 3 mm×3 mm on the substrate, and then washed. After the substrate was mounted on a spin coater, PEDOT:PSS was spin-coated on the ITO layer with a thickness of 50 nm. Thereafter, the solvent was removed by drying on a hot plate at 150° C. for 10 minutes. Then, the present inventive compound P-97 as a hole transport material dissolved in xylene was spin-coated with a thickness of 30 nm. After drying on a hot plate at 100° C. for 10 minutes, the hole transport material was crosslinked by heating at 200° C. for 30 minutes. The hole transport layer was doped with AND as a host material and DPAVBi as a dopant material at 96:4 for a light emitting layer, and then a solution dissolved in xylene was spin-coated with a thickness of 30 nm, and dried on a hot plate at 100° C. for 10 minutes, followed by mounting on a vacuum chamber, and subjected to a base pressure of 1×10⁻⁶ torr.

Subsequently, (1,1′-bisphenyl)-4-olato)bis(2-methyl-8-quinolinolato)aluminum was vacuum-deposited with a thickness of 10 nm for a hole blocking layer, and tris(8-quinolinol)aluminum was formed with a thickness of 40 nm for an electron injection layer. Thereafter, LiF, which is a halogenated alkali metal, was deposited with a thickness of 0.5 nm for an electron injection layer, and subsequently Al was deposited with a thickness of 150 nm and used as a cathode. In this way, the organic light emitting diode was manufactured.

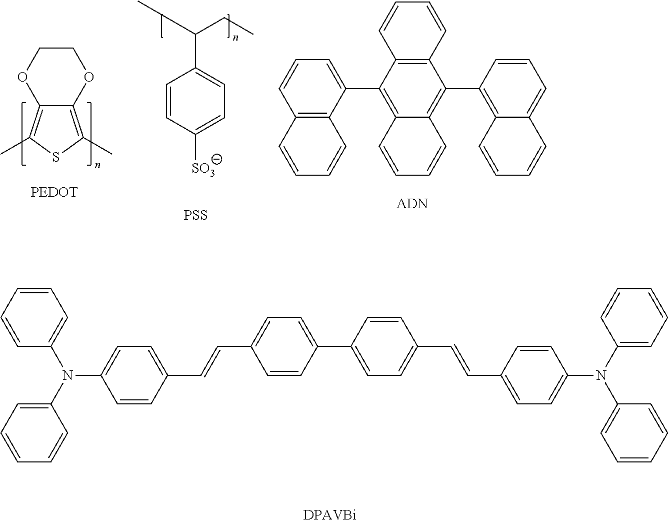

### Example 47

An organic light emitting diode was manufactured by the same method as in Example 46 except that as a material for the electron transport layer, compound P-98 of the present disclosure shown in table 4 below was used instead of Compound P-97 of the present disclosure.

### Comparative Example 4

An organic light emitting diode was manufactured by the same method as in Example 46 except that as a material for the electron transport layer, Comparative Compound 3 was used instead of Compound P-97 of the present disclosure.

### Comparative Compound 3

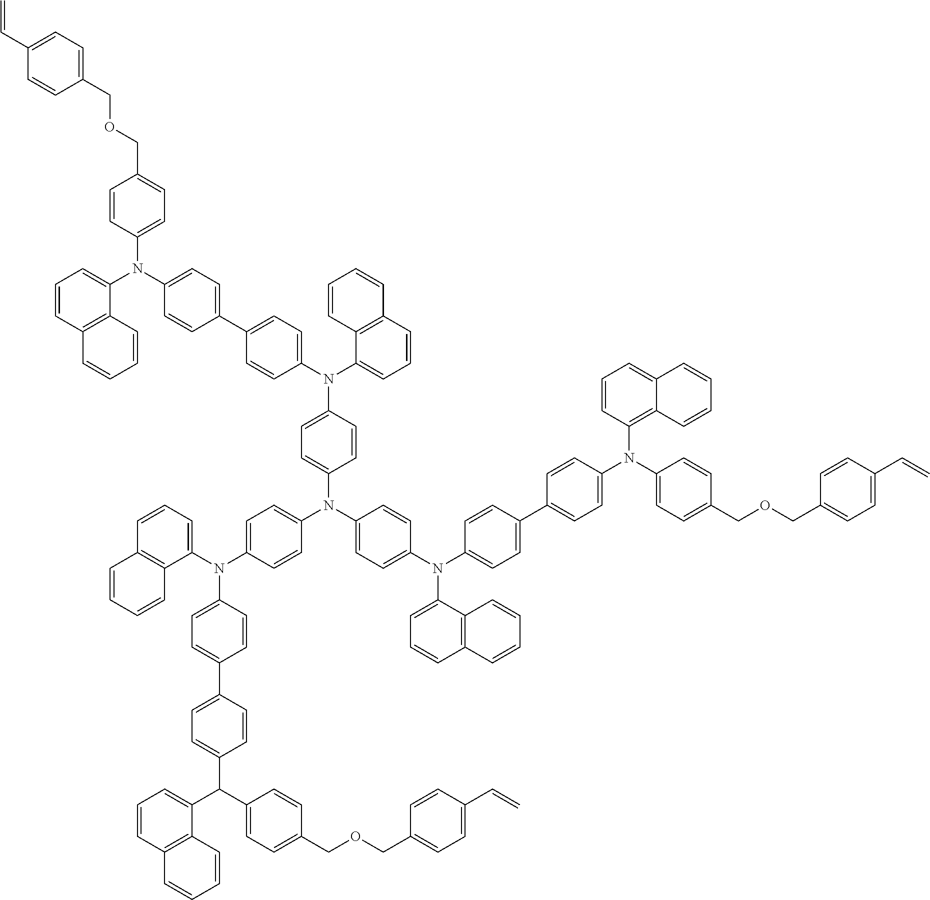 [SMILES: C=Cc1ccc(COCc2ccc(C(c3ccc(-c4ccc(N(c5ccc(N(c6ccc(N(c7ccc(-c8ccc(N(c9ccc(COCc%10ccc(C=C)cc%10)cc9)c9cccc%10ccccc9%10)cc8)cc7)c7cccc8ccccc78)cc6)c6ccc(N(c7ccc(-c8ccc(N(c9ccc(COCc%10ccc(C=C)cc%10)cc9)c9cccc%10ccccc9%10)cc8)cc7)c7cccc8ccccc78)cc6)cc5)c5cccc6ccccc56)cc4)cc3)c3cccc4ccccc34)cc2)cc1]

A forward bias DC voltage was applied to each of the organic light emitting diodes manufactured in Examples 46 and 47 and Comparative example 4 to measure electro-luminescent (EL) characteristics thereof by PR-650 (Photoresearch). As a result, the T95 lifetime was measured by a lifetime measurement equipment (fabricated by Mcscience) at reference brightness of 5000 cd/m². The element manufacturing and evaluation results are shown in the table below.

TABLE 5

| | | | Current | Bright- | Effi- | Life- |
|---|---|---|---|---|---|---|
| | Compound | Voltage | Density | ness | ciency | time |
| Comparative | Comparative | 5.5 | 12.4 | 500 | 4.0 | 78.0 |
| Example 4 | Compound 3 | | | | | |
| Example 46 | Compound | 4.9 | 8.2 | 500 | 6.1 | 96.6 |
| | (P-97) | | | | | |
| Example 47 | Compound | 4.8 | 7.6 | 500 | 6.6 | 117.8 |
| | (P-98) | | | | | |

As can be seen from the results of Table 5 above, compared with the organic light emitting diode using Comparative Compound 3 as a material for the hole transport layer, the organic light emitting diodes using each of the present inventive compounds as a material for the hole transport layer showed significantly improved driving voltage, light emission efficiency, and lifetime.

In other words, compared with the element using, as a material for the hole transport layer, Comparative Compound 3 having a structure in which a crosslinking substance is linked to the end of the NPB derivative, the elements using, as a material for the hole transport layer, the compounds of the present disclosure having a 5-membered ring and 4-spirofluorne showed a low driving voltage, a high efficiency, and a long lifetime.

The reason why the elements using the compounds of the present disclosure as a material for the light emitting layer showed a low driving voltage and a high efficiency as described above is considered to be that the HOMO or LUMO energy levels of the compounds of the present disclosure have appropriate values between the hole transport layer and the light emitting layer, results in a charge balance of holes and electrons, and thus light emission occurs inside the light emitting layer but not in the interface of the hole transport layer, thereby attaining higher efficiency and maximizing lifetime.

Although exemplary embodiments of the present disclosure have been described for illustrative purposes, a person skilled in the art will appreciate that various modifications, additions, and substitutions are possible, without departing from the scope and spirit of the invention as disclosed in the accompanying claims. Therefore, the embodiment disclosed in the present disclosure is intended to illustrate the scope of the technical idea of the present disclosure, and the scope of the present disclosure is not limited by the embodiment. The scope of the present disclosure shall be construed on the basis of the accompanying claims, and it shall be construed that all of the technical ideas included within the scope equivalent to the claims belong to the present disclosure.

### CROSS-REFERENCE TO RELATED APPLICATION

This patent application claims priorities under 35 U.S.C. § 119(a) on Korean Patent Application No. 10-2017-0033368 filed on 16 Mar. 2017 and No. 10-2018-0001465 filed on 5 Jan. 2018, the disclosure of which is incorporated herein by reference. In addition, this patent application claims priorities in countries other than U.S., with the same reason based on the Korean Patent Application, the entire contents of which are incorporated herein by reference.

1. A compound represented by Formula 1: 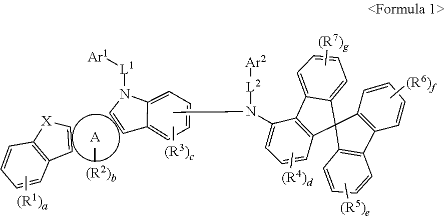
in Formula 1,
1) Ar¹ and Ar² each are independently the same as or different from each other, and selected from the group consisting of a C₆-C₆₀ aryl group, a fluorenyl group, a C₂-C₆₀ heterocyclic group containing at least one heteroatom of O, N, S, Si, and P, —N(R^{a})(R^{b}), a fused ring group of a C₆-C₆₀ aromatic ring and a C₃-C₆₀ aliphatic ring, a C₁-C₅₀ alkyl group, a C₂-C₂₀ alkenyl group, a C₂-C₂₀ alkynyl group, a C₁-C₃₀ alkoxy group, and a C₆-C₃₀ aryloxy group (with the proviso that Ar¹ may not be a heteroaryl containing N);
2) X is any one of N-L³-Ar³, O, S, Se, Ge, and SiR^{c}R^{d};
3) R¹ to R⁷ each are independently the same as or different from each other, and selected from the group consisting of deuterium, tritium, halogen, a cyano group, a nitro group, a C₆-C₆₀ aryl group, a fluorenyl group, a C₂-C₆₀ heterocyclic group containing at least one heteroatom selected from the group consisting of O, N, S, Si, and P, a fused ring group of a C₃-C₆₀ aliphatic ring and a C₆-C₆₀ aromatic ring, a C₁-C₅₀ alkyl group, a C₂-C₂₀ alkenyl group, a C₂-C₂₀ alkynyl group, a C₁-C₃₀ alkoxy group, and a C₆-C₃₀ aryloxy group, wherein R¹ to R⁷ may bind to each other to form a ring (in the presence of a plurality of R¹'s to R⁷'s, at least one pair of independently neighboring R¹'s, R²'s, R³'s, R⁴'s, R⁵'s, R⁶'s, and R⁷'s may bind to each other to form a ring, provided that R¹'s to R⁷'s forming no ring are the same as defined above);
4) a, e, f, and g are an integer of 0 to 4, b is an integer of 0 to 2, and d is an integer of 0 to 3;
5) ring A is a C₆ aryl group;
6) L¹ to L³ each are selected from the group consisting of a direct bond, a C₆-C₆₀ arylene group, a C₂-C₆₀ heterocyclic group containing at least one heteroatom selected from the group consisting of O, N, S, Si, and P, a fluorenylene group, a divalent fused ring of a C₃-C₆₀ aliphatic ring and a C₆-C₆₀ aromatic ring, and a C₁-C₆₀ aliphatic hydrocarbon group;
7) Ar³ is selected from the group consisting of a C₆-C₆₀ aryl group, a fluorenyl group, a C₂-C₆₀ heterocyclic group containing at least one heteroatom selected from the group consisting of O, N, S, Si, and P, —N(R^{a})(R^{b}), a fused ring group of a C₆-C₆₀ aromatic ring and a C₃-C₆₀ aliphatic ring, a C₁-C₅₀ alkyl group, a C₂-C₂₀ alkenyl group, a C₂-C₂₀ alkynyl group, a C₁-C₃₀ alkoxy group, and a C₆-C₃₀ aryloxy group; and
8) R^{a}, R^{b}, R^{c}, and R^{d} each are independently selected from the group consisting of deuterium, tritium, halogen, a cyano group, a nitro group, a C₆-C₆₀ aryl group, a fluorenyl group, a C₂-C₆₀ heterocyclic group containing at least one heteroatom selected from the group consisting of O, N, S, Si, and P, a fused ring group of a C₃-C₆₀ aliphatic ring and a C₆-C₆₀ aromatic ring, a C₁-C₅₀ alkyl group, a C₂-C₂₀ alkenyl group, a C₂-C₂₀ alkynyl group, a C₁-C₃₀ alkoxy group, and a C₆-C₃₀ aryloxy group (wherein R^{c} and R^{d} may form a spiro compound by forming a ring),
wherein the aryl group, fluorenylene group, fluorenyl group, heterocyclic group, alkyl group, fused ring group, alkenyl group, alkoxy group, and aryloxy group each may be further substituted with at least one substituent selected from the group consisting of deuterium, halogen, a silane group substituted or unsubstituted with a C₁-C₂₀ alkyl group or a C₆-C₂₀ aryl group, a siloxane group, a boron group, a germanium group, a cyano group, a nitro group, —N(R^{e})(R^{f}) (here, R^{e} and R^{f} are the same as the above-described definition of R^{a} and R^{d}, respectively), a C₁-C₂₀ alkylthio group, a C₁-C₂₀ alkoxy group, a C₁-C₂₀ alkyl group, a C₂-C₂₀ alkenyl group, a C₂-C₂₀ alkynyl group, a C₆-C₂₀ aryl group, a C₆-C₂₀ aryl group substituted with deuterium, a fluorenyl group, a C₂-C₂₀ heterocyclic group containing at least one heteroatom selected from the group consisting of O, N, S, Si, and P, a C₃-C₂₀ cycloalkyl group, a C₇-C₂₀ arylalkyl group, and a C₈-C₂₀ arylalkenyl group, and when those substituents are adjacent, the substituents may bind to each other to form a ring.

2. The compound of claim 1, wherein the compound represented by Formula 1 above is represented by one of Formulas 2 to 7 below: 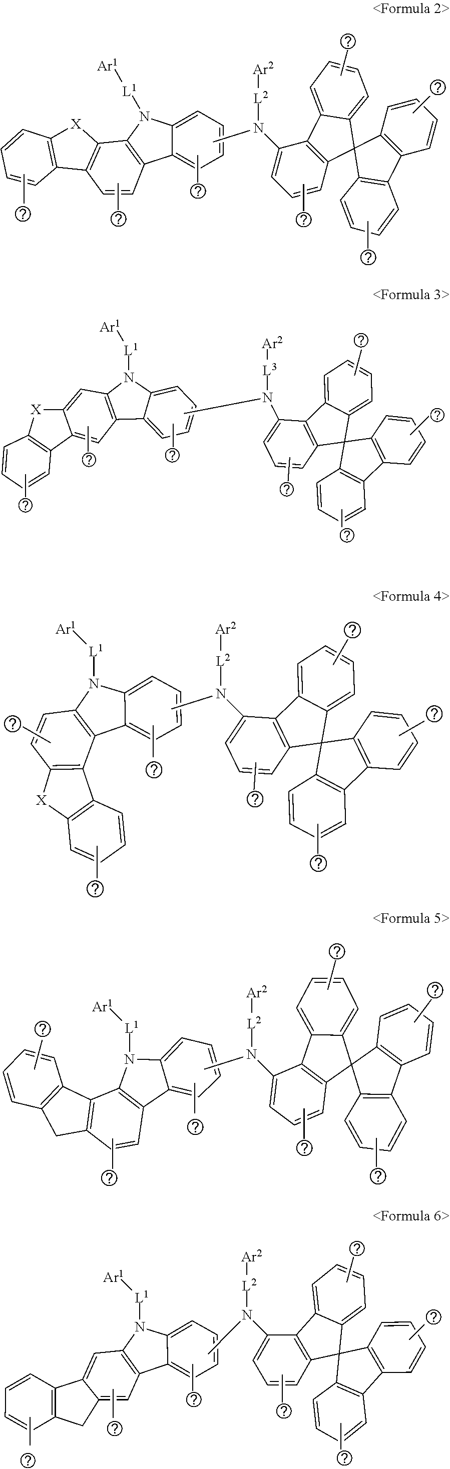 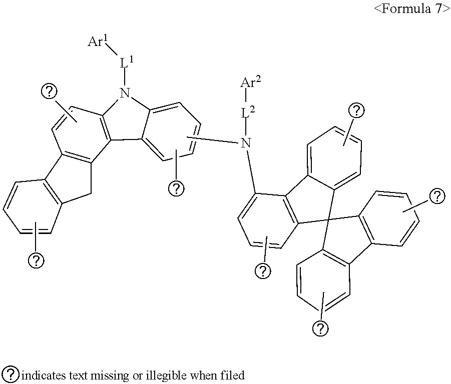
in Formulas 2 to 7,
X, L¹, L², Ar¹, Ar², R¹ to R⁷, and a to f are the same as X, L¹, L², Ar¹, Ar², R¹ to R⁷, and a to f defined in Formula 1 above, respectively.

3. The compound of claim 1, wherein Formula 1 above is represented by one of Formulas P-1 to P-112 below:

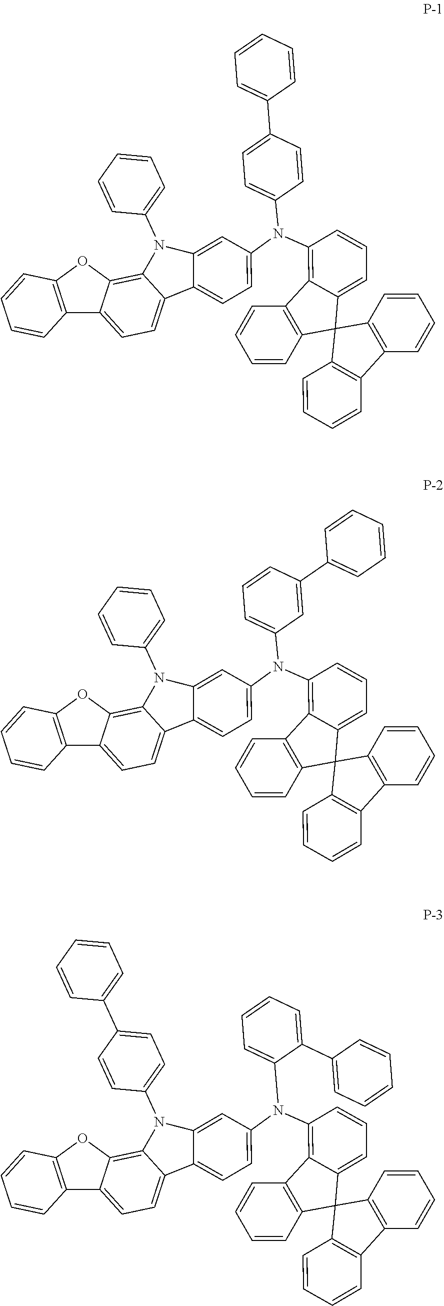 [SMILES: c1ccc(-c2ccc(N(c3ccc4c5ccc6c7ccccc7oc6c5n(-c5ccccc5)c4c3)c3cccc4c3-c3ccccc3C43c4ccccc4-c4ccccc43)cc2)cc1 ; c1ccc(-c2cccc(N(c3ccc4c5ccc6c7ccccc7oc6c5n(-c5ccccc5)c4c3)c3cccc4c3-c3ccccc3C43c4ccccc4-c4ccccc43)c2)cc1 ; c1ccc(-c2ccc(-n3c4cc(N(c5ccccc5-c5ccccc5)c5cccc6c5-c5ccccc5C65c6ccccc6-c6ccccc65)ccc4c4ccc5c6ccccc6oc5c43)cc2)cc1]

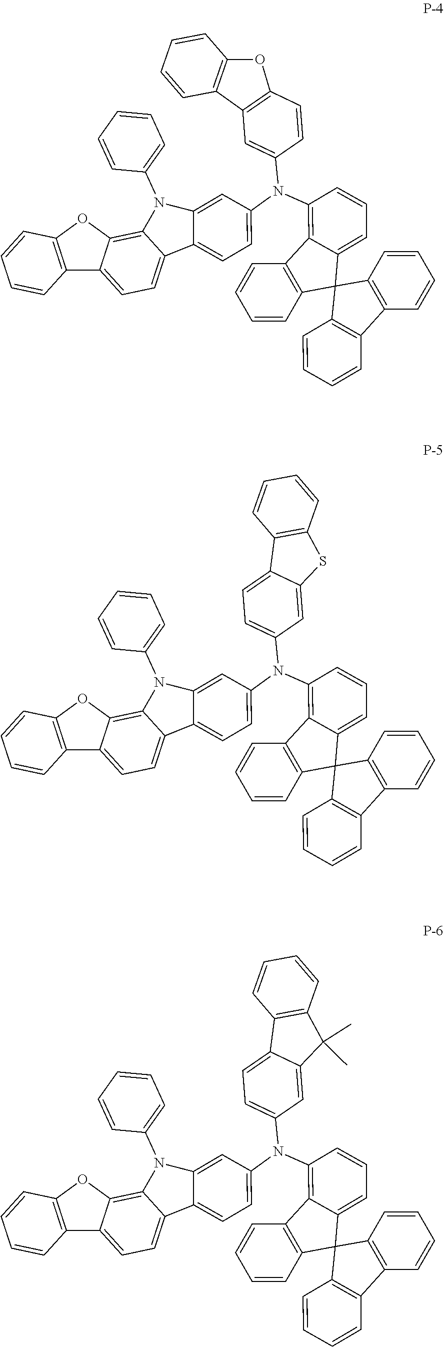 [SMILES: c1ccc(-n2c3cc(N(c4ccc5oc6ccccc6c5c4)c4cccc5c4-c4ccccc4C54c5ccccc5-c5ccccc54)ccc3c3ccc4c5ccccc5oc4c32)cc1 ; c1ccc(-n2c3cc(N(c4ccc5c(c4)sc4ccccc45)c4cccc5c4-c4ccccc4C54c5ccccc5-c5ccccc54)ccc3c3ccc4c5ccccc5oc4c32)cc1 ; CC1(C)c2ccccc2-c2ccc(N(c3ccc4c5ccc6c7ccccc7oc6c5n(-c5ccccc5)c4c3)c3cccc4c3-c3ccccc3C43c4ccccc4-c4ccccc43)cc21]

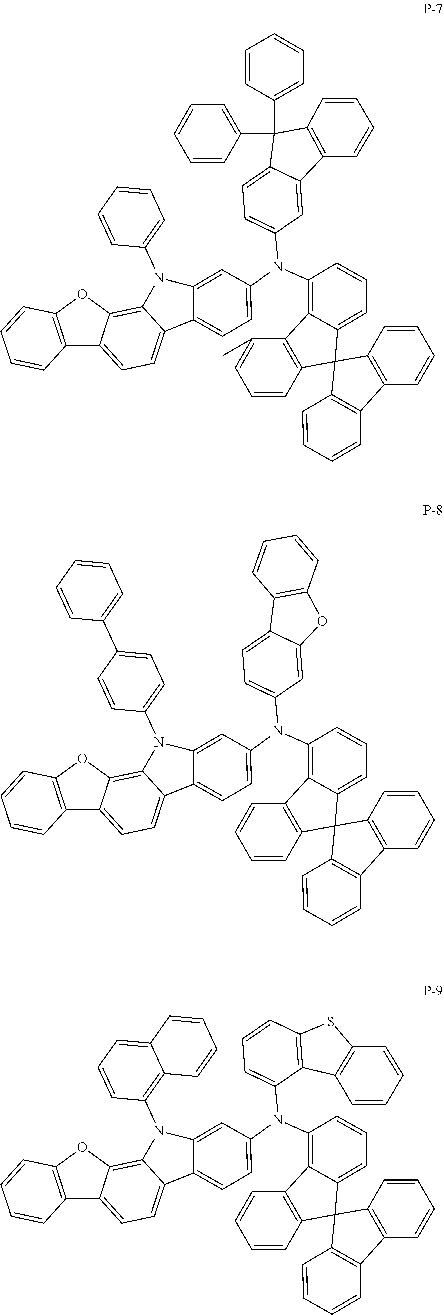 [SMILES: Cc1cccc2c1-c1c(N(c3ccc4c(c3)-c3ccccc3C4(c3ccccc3)c3ccccc3)c3ccc4c5ccc6c7ccccc7oc6c5n(-c5ccccc5)c4c3)cccc1C21c2ccccc2-c2ccccc21 ; c1ccc(-c2ccc(-n3c4cc(N(c5ccc6c(c5)oc5ccccc56)c5cccc6c5-c5ccccc5C65c6ccccc6-c6ccccc65)ccc4c4ccc5c6ccccc6oc5c43)cc2)cc1 ; c1ccc2c(c1)-c1ccccc1C21c2ccccc2-c2c(N(c3ccc4c5ccc6c7ccccc7oc6c5n(-c5cccc6ccccc56)c4c3)c3cccc4sc5ccccc5c34)cccc21]

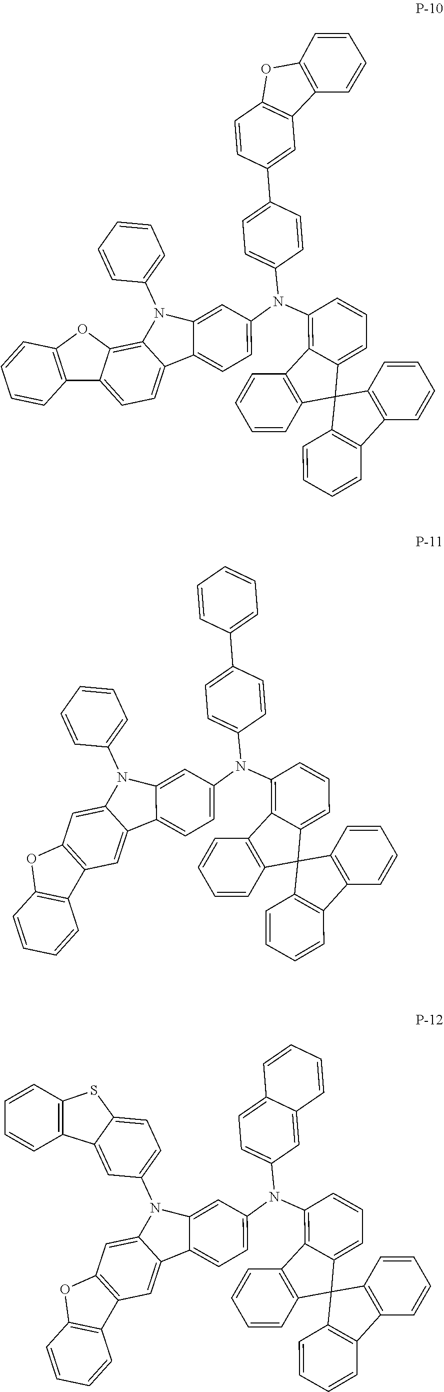 [SMILES: c1ccc(-n2c3cc(N(c4ccc(-c5ccc6oc7ccccc7c6c5)cc4)c4cccc5c4-c4ccccc4C54c5ccccc5-c5ccccc54)ccc3c3ccc4c5ccccc5oc4c32)cc1 ; c1ccc(-c2ccc(N(c3ccc4c5cc6c(cc5n(-c5ccccc5)c4c3)oc3ccccc36)c3cccc4c3-c3ccccc3C43c4ccccc4-c4ccccc43)cc2)cc1 ; c1ccc2c(c1)-c1ccccc1C21c2ccccc2-c2c(N(c3ccc4ccccc4c3)c3ccc4c5cc6c(cc5n(-c5ccc7sc8ccccc8c7c5)c4c3)oc3ccccc36)cccc21]

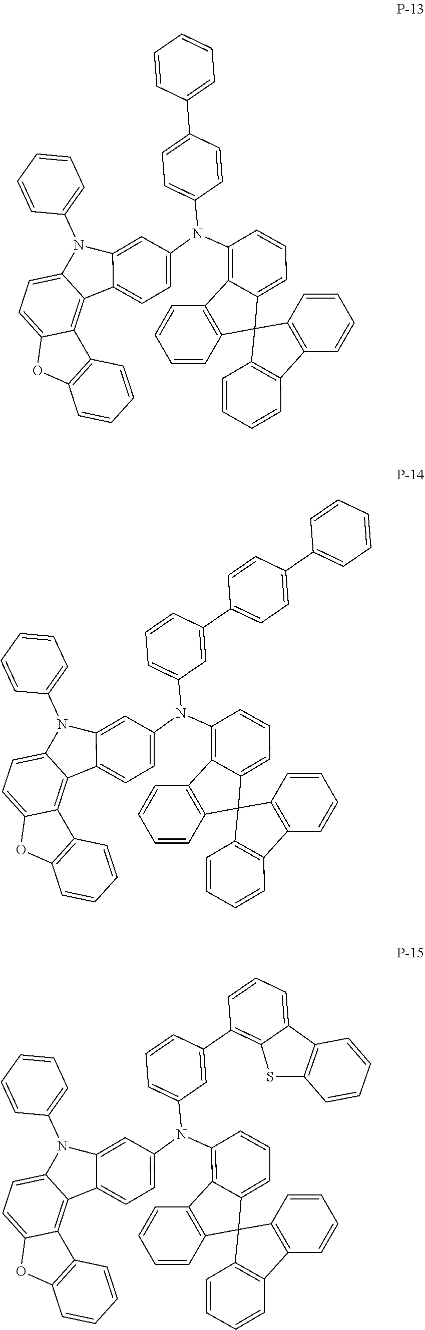 [SMILES: c1ccc(-c2ccc(N(c3ccc4c5c6c(ccc5n(-c5ccccc5)c4c3)oc3ccccc36)c3cccc4c3-c3ccccc3C43c4ccccc4-c4ccccc43)cc2)cc1 ; c1ccc(-c2ccc(-c3cccc(N(c4ccc5c6c7c(ccc6n(-c6ccccc6)c5c4)oc4ccccc47)c4cccc5c4-c4ccccc4C54c5ccccc5-c5ccccc54)c3)cc2)cc1 ; c1ccc(-n2c3cc(N(c4cccc(-c5cccc6c5sc5ccccc56)c4)c4cccc5c4-c4ccccc4C54c5ccccc5-c5ccccc54)ccc3c3c4c(ccc32)oc2ccccc24)cc1]

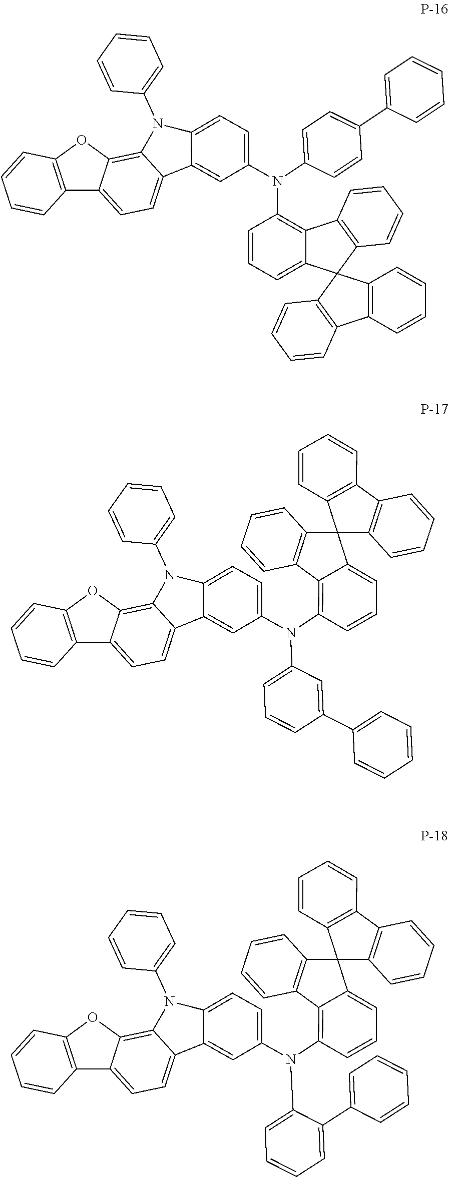 [SMILES: c1ccc(-c2ccc(N(c3ccc4c(c3)c3ccc5c6ccccc6oc5c3n4-c3ccccc3)c3cccc4c3-c3ccccc3C43c4ccccc4-c4ccccc43)cc2)cc1 ; c1ccc(-c2cccc(N(c3ccc4c(c3)c3ccc5c6ccccc6oc5c3n4-c3ccccc3)c3cccc4c3-c3ccccc3C43c4ccccc4-c4ccccc43)c2)cc1 ; c1ccc(-c2ccccc2N(c2ccc3c(c2)c2ccc4c5ccccc5oc4c2n3-c2ccccc2)c2cccc3c2-c2ccccc2C32c3ccccc3-c3ccccc32)cc1]

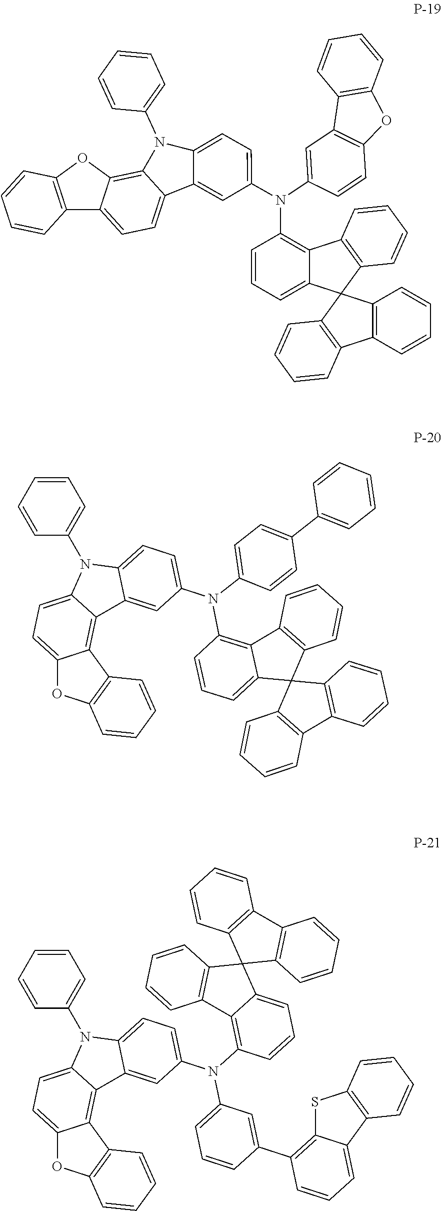 [SMILES: c1ccc(-n2c3ccc(N(c4ccc5oc6ccccc6c5c4)c4cccc5c4-c4ccccc4C54c5ccccc5-c5ccccc54)cc3c3ccc4c5ccccc5oc4c32)cc1 ; c1ccc(-c2ccc(N(c3ccc4c(c3)c3c5c(ccc3n4-c3ccccc3)oc3ccccc35)c3cccc4c3-c3ccccc3C43c4ccccc4-c4ccccc43)cc2)cc1 ; c1ccc(-n2c3ccc(N(c4cccc(-c5cccc6c5sc5ccccc56)c4)c4cccc5c4-c4ccccc4C54c5ccccc5-c5ccccc54)cc3c3c4c(ccc32)oc2ccccc24)cc1]

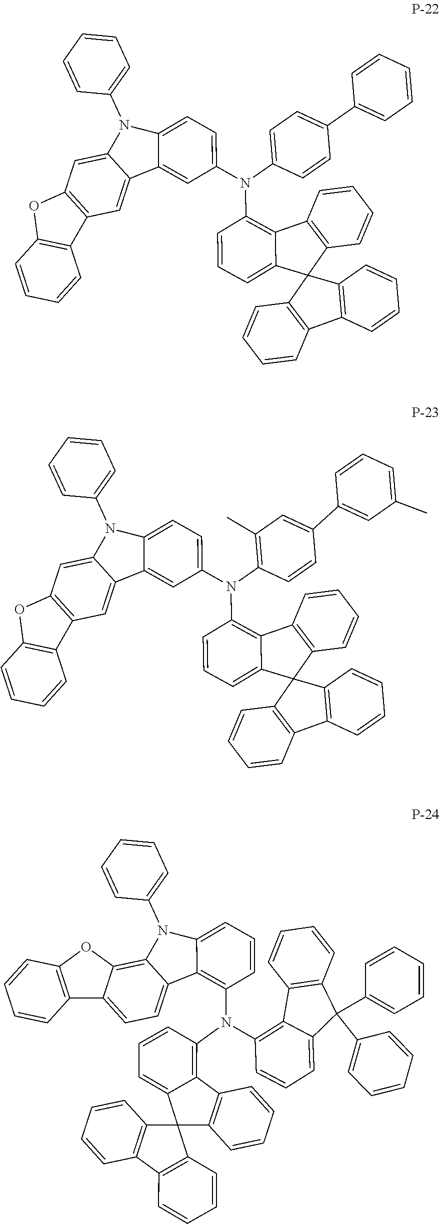 [SMILES: c1ccc(-c2ccc(N(c3ccc4c(c3)c3cc5c(cc3n4-c3ccccc3)oc3ccccc35)c3cccc4c3-c3ccccc3C43c4ccccc4-c4ccccc43)cc2)cc1 ; Cc1cccc(-c2ccc(N(c3ccc4c(c3)c3cc5c(cc3n4-c3ccccc3)oc3ccccc35)c3cccc4c3-c3ccccc3C43c4ccccc4-c4ccccc43)c(C)c2)c1 ; c1ccc(-n2c3cccc(N(c4cccc5c4-c4ccccc4C5(c4ccccc4)c4ccccc4)c4cccc5c4-c4ccccc4C54c5ccccc5-c5ccccc54)c3c3ccc4c5ccccc5oc4c32)cc1]

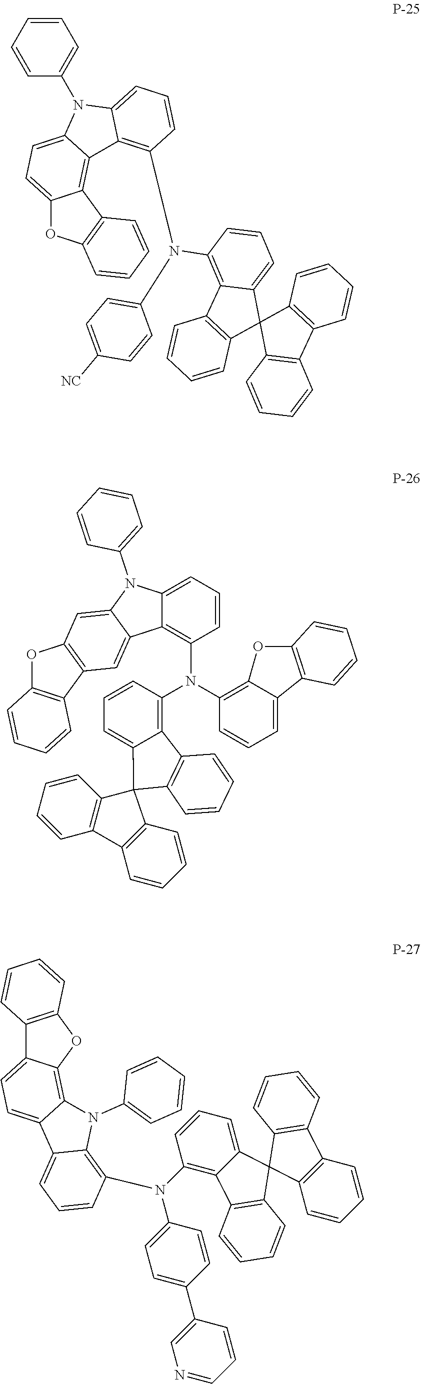 [SMILES: N#Cc1ccc(N(c2cccc3c2-c2ccccc2C32c3ccccc3-c3ccccc32)c2cccc3c2c2c4c(ccc2n3-c2ccccc2)oc2ccccc24)cc1 ; c1ccc(-n2c3cc4oc5ccccc5c4cc3c3c(N(c4cccc5c4-c4ccccc4C54c5ccccc5-c5ccccc54)c4cccc5c4oc4ccccc45)cccc32)cc1 ; c1ccc(-n2c3c(N(c4ccc(-c5cccnc5)cc4)c4cccc5c4-c4ccccc4C54c5ccccc5-c5ccccc54)cccc3c3ccc4c5ccccc5oc4c32)cc1]

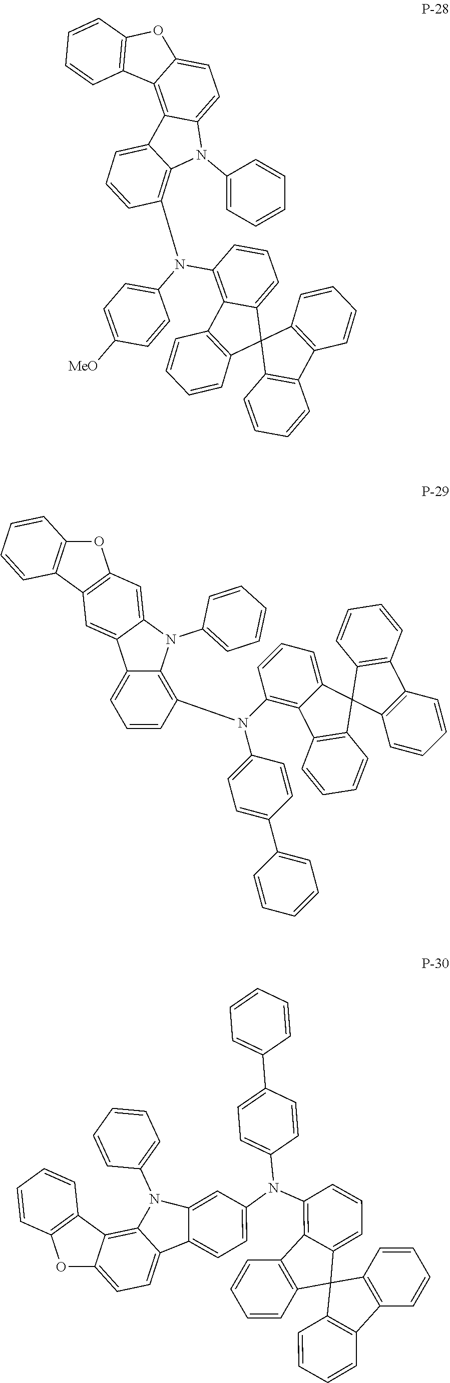 [SMILES: COc1ccc(N(c2cccc3c2-c2ccccc2C32c3ccccc3-c3ccccc32)c2cccc3c4c5c(ccc4n(-c4ccccc4)c23)oc2ccccc25)cc1 ; c1ccc(-c2ccc(N(c3cccc4c3-c3ccccc3C43c4ccccc4-c4ccccc43)c3cccc4c5cc6c(cc5n(-c5ccccc5)c34)oc3ccccc36)cc2)cc1 ; c1ccc(-c2ccc(N(c3ccc4c5ccc6oc7ccccc7c6c5n(-c5ccccc5)c4c3)c3cccc4c3-c3ccccc3C43c4ccccc4-c4ccccc43)cc2)cc1]

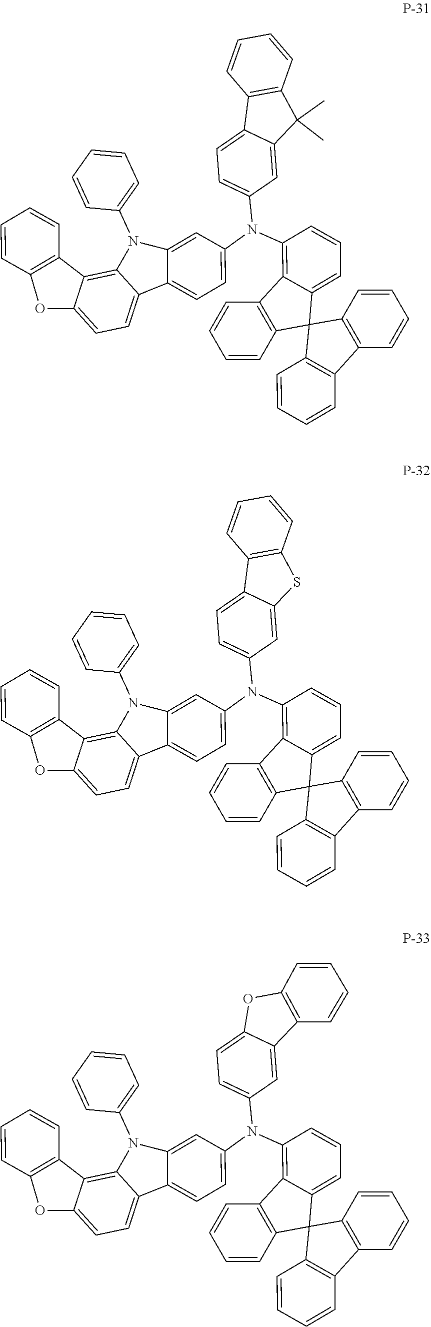 [SMILES: CC1(C)c2ccccc2-c2ccc(N(c3ccc4c5ccc6oc7ccccc7c6c5n(-c5ccccc5)c4c3)c3cccc4c3-c3ccccc3C43c4ccccc4-c4ccccc43)cc21 ; c1ccc(-n2c3cc(N(c4ccc5c(c4)sc4ccccc45)c4cccc5c4-c4ccccc4C54c5ccccc5-c5ccccc54)ccc3c3ccc4oc5ccccc5c4c32)cc1 ; c1ccc(-n2c3cc(N(c4ccc5oc6ccccc6c5c4)c4cccc5c4-c4ccccc4C54c5ccccc5-c5ccccc54)ccc3c3ccc4oc5ccccc5c4c32)cc1]

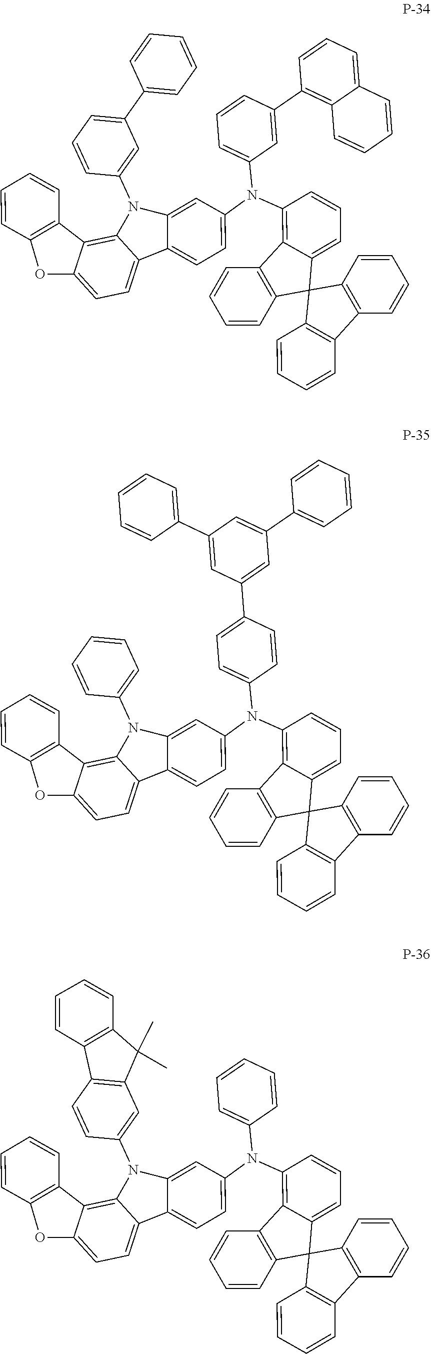 [SMILES: c1ccc(-c2cccc(-n3c4cc(N(c5cccc(-c6cccc7ccccc67)c5)c5cccc6c5-c5ccccc5C65c6ccccc6-c6ccccc65)ccc4c4ccc5oc6ccccc6c5c43)c2)cc1 ; c1ccc(-c2cc(-c3ccccc3)cc(-c3ccc(N(c4ccc5c6ccc7oc8ccccc8c7c6n(-c6ccccc6)c5c4)c4cccc5c4-c4ccccc4C54c5ccccc5-c5ccccc54)cc3)c2)cc1 ; CC1(C)c2ccccc2-c2ccc(-n3c4cc(N(c5ccccc5)c5cccc6c5-c5ccccc5C65c6ccccc6-c6ccccc65)ccc4c4ccc5oc6ccccc6c5c43)cc21]

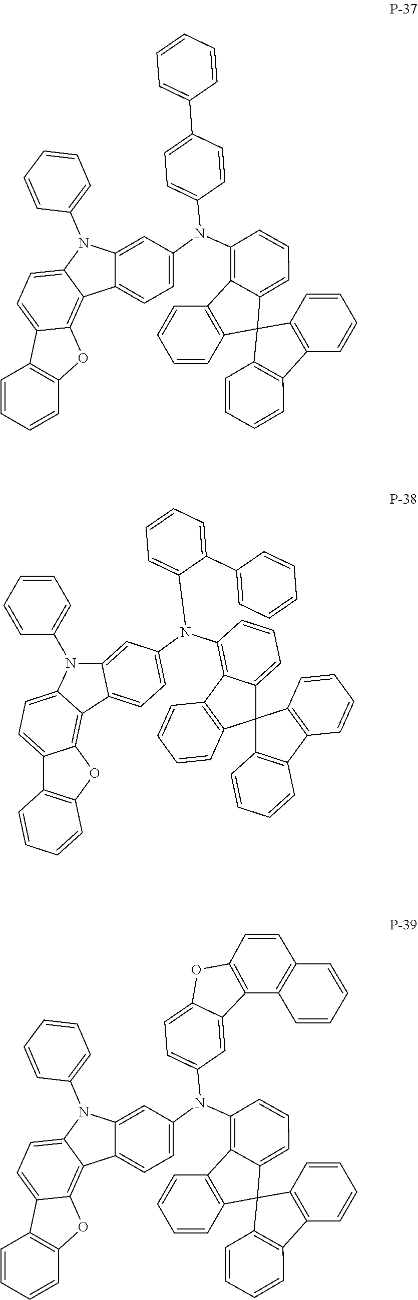 [SMILES: c1ccc(-c2ccc(N(c3ccc4c5c6oc7ccccc7c6ccc5n(-c5ccccc5)c4c3)c3cccc4c3-c3ccccc3C43c4ccccc4-c4ccccc43)cc2)cc1 ; c1ccc(-c2ccccc2N(c2ccc3c4c5oc6ccccc6c5ccc4n(-c4ccccc4)c3c2)c2cccc3c2-c2ccccc2C32c3ccccc3-c3ccccc32)cc1 ; c1ccc(-n2c3cc(N(c4ccc5oc6ccc7ccccc7c6c5c4)c4cccc5c4-c4ccccc4C54c5ccccc5-c5ccccc54)ccc3c3c4oc5ccccc5c4ccc32)cc1]

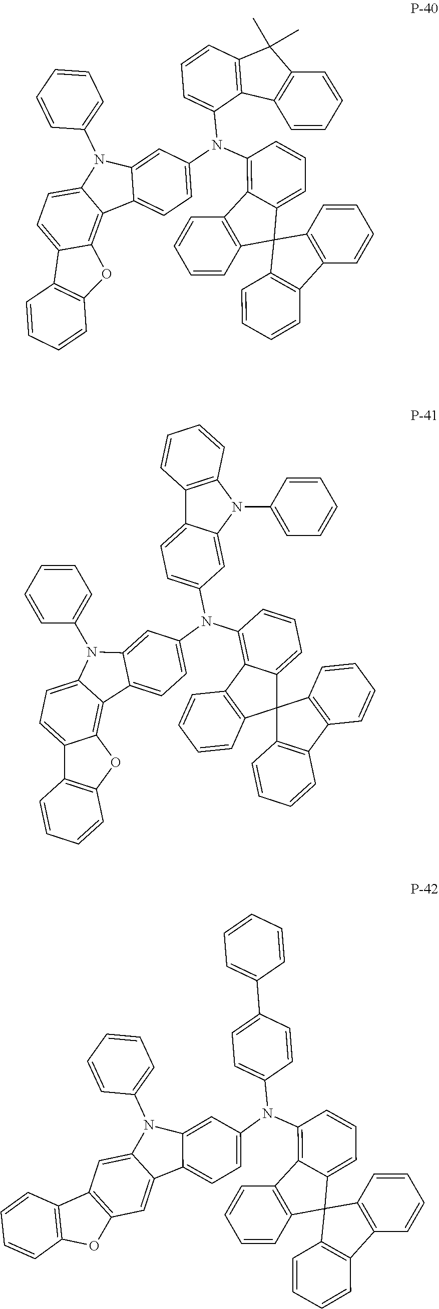 [SMILES: CC1(C)c2ccccc2-c2c(N(c3ccc4c5c6oc7ccccc7c6ccc5n(-c5ccccc5)c4c3)c3cccc4c3-c3ccccc3C43c4ccccc4-c4ccccc43)cccc21 ; c1ccc(-n2c3ccccc3c3ccc(N(c4ccc5c6c7oc8ccccc8c7ccc6n(-c6ccccc6)c5c4)c4cccc5c4-c4ccccc4C54c5ccccc5-c5ccccc54)cc32)cc1 ; c1ccc(-c2ccc(N(c3ccc4c5cc6oc7ccccc7c6cc5n(-c5ccccc5)c4c3)c3cccc4c3-c3ccccc3C43c4ccccc4-c4ccccc43)cc2)cc1]

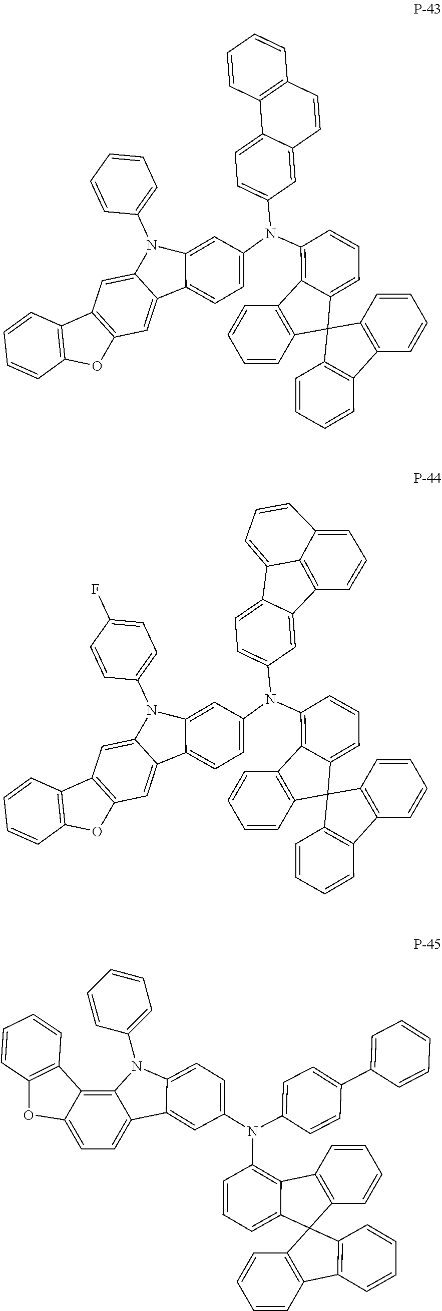 [SMILES: c1ccc(-n2c3cc(N(c4ccc5c(ccc6ccccc65)c4)c4cccc5c4-c4ccccc4C54c5ccccc5-c5ccccc54)ccc3c3cc4oc5ccccc5c4cc32)cc1 ; Fc1ccc(-n2c3cc(N(c4ccc5c(c4)-c4cccc6cccc-5c46)c4cccc5c4-c4ccccc4C54c5ccccc5-c5ccccc54)ccc3c3cc4oc5ccccc5c4cc32)cc1 ; c1ccc(-c2ccc(N(c3ccc4c(c3)c3ccc5oc6ccccc6c5c3n4-c3ccccc3)c3cccc4c3-c3ccccc3C43c4ccccc4-c4ccccc43)cc2)cc1]

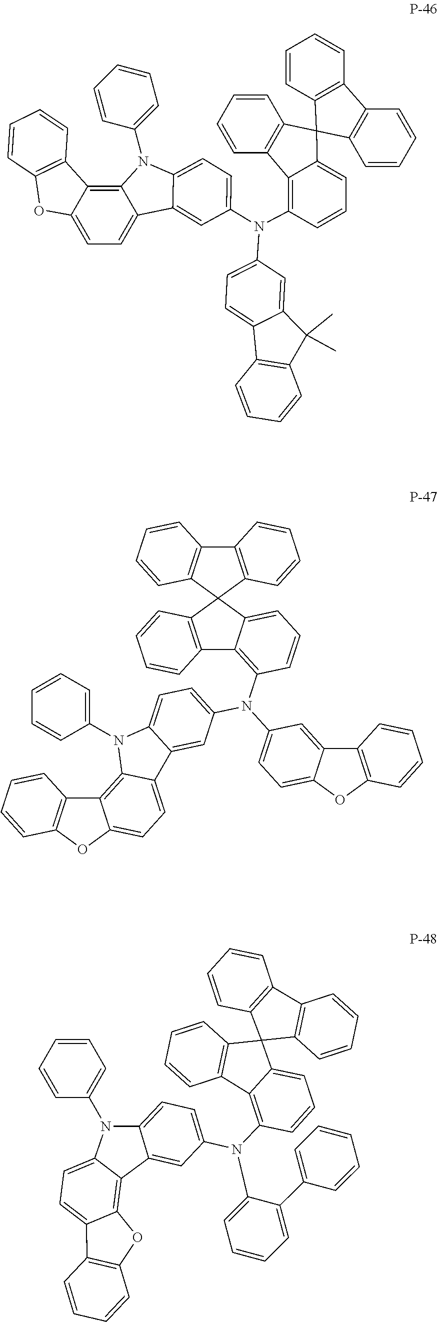 [SMILES: CC1(C)c2ccccc2-c2ccc(N(c3ccc4c(c3)c3ccc5oc6ccccc6c5c3n4-c3ccccc3)c3cccc4c3-c3ccccc3C43c4ccccc4-c4ccccc43)cc21 ; c1ccc(-n2c3ccc(N(c4ccc5oc6ccccc6c5c4)c4cccc5c4-c4ccccc4C54c5ccccc5-c5ccccc54)cc3c3ccc4oc5ccccc5c4c32)cc1 ; c1ccc(-c2ccccc2N(c2ccc3c(c2)c2c4oc5ccccc5c4ccc2n3-c2ccccc2)c2cccc3c2-c2ccccc2C32c3ccccc3-c3ccccc32)cc1]

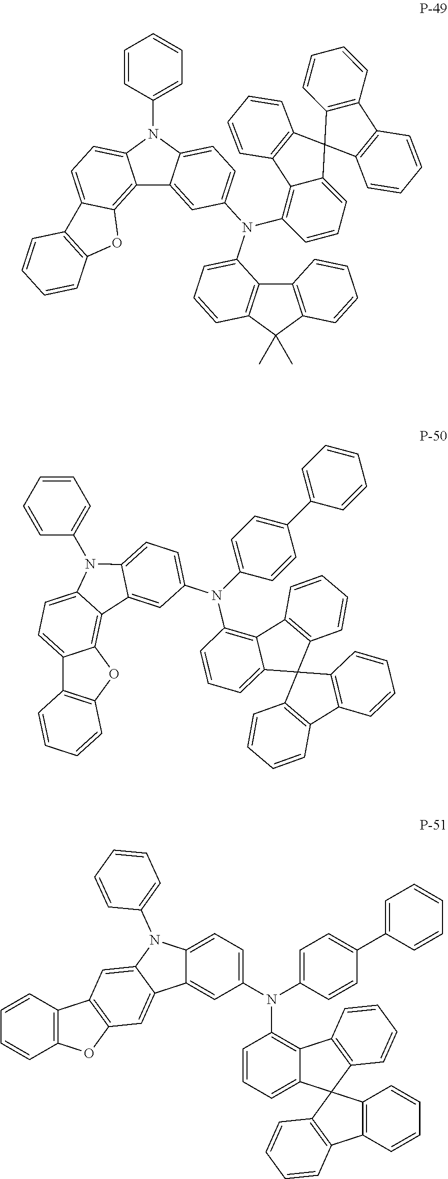 [SMILES: CC1(C)c2ccccc2-c2c(N(c3ccc4c(c3)c3c5oc6ccccc6c5ccc3n4-c3ccccc3)c3cccc4c3-c3ccccc3C43c4ccccc4-c4ccccc43)cccc21 ; c1ccc(-c2ccc(N(c3ccc4c(c3)c3c5oc6ccccc6c5ccc3n4-c3ccccc3)c3cccc4c3-c3ccccc3C43c4ccccc4-c4ccccc43)cc2)cc1 ; c1ccc(-c2ccc(N(c3ccc4c(c3)c3cc5oc6ccccc6c5cc3n4-c3ccccc3)c3cccc4c3-c3ccccc3C43c4ccccc4-c4ccccc43)cc2)cc1]

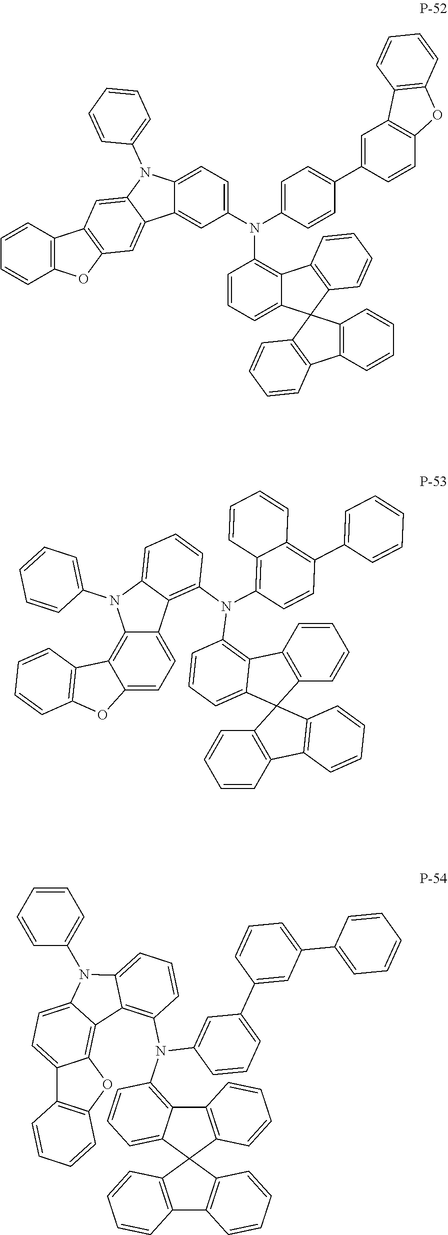 [SMILES: c1ccc(-n2c3ccc(N(c4ccc(-c5ccc6oc7ccccc7c6c5)cc4)c4cccc5c4-c4ccccc4C54c5ccccc5-c5ccccc54)cc3c3cc4oc5ccccc5c4cc32)cc1 ; c1ccc(-c2ccc(N(c3cccc4c3-c3ccccc3C43c4ccccc4-c4ccccc43)c3cccc4c3c3ccc5oc6ccccc6c5c3n4-c3ccccc3)c3ccccc23)cc1 ; c1ccc(-c2cccc(-c3cccc(N(c4cccc5c4-c4ccccc4C54c5ccccc5-c5ccccc54)c4cccc5c4c4c6oc7ccccc7c6ccc4n5-c4ccccc4)c3)c2)cc1]

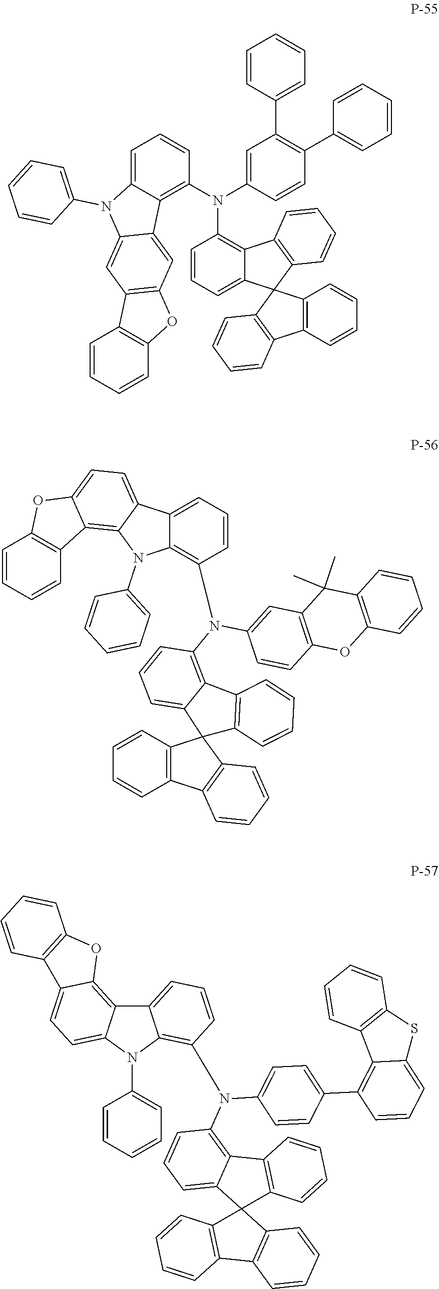 [SMILES: c1ccc(-c2ccc(N(c3cccc4c3-c3ccccc3C43c4ccccc4-c4ccccc43)c3cccc4c3c3cc5oc6ccccc6c5cc3n4-c3ccccc3)cc2-c2ccccc2)cc1 ; CC1(C)c2ccccc2Oc2ccc(N(c3cccc4c3-c3ccccc3C43c4ccccc4-c4ccccc43)c3cccc4c5ccc6oc7ccccc7c6c5n(-c5ccccc5)c34)cc21 ; c1ccc(-n2c3ccc4c5ccccc5oc4c3c3cccc(N(c4ccc(-c5cccc6sc7ccccc7c56)cc4)c4cccc5c4-c4ccccc4C54c5ccccc5-c5ccccc54)c32)cc1]

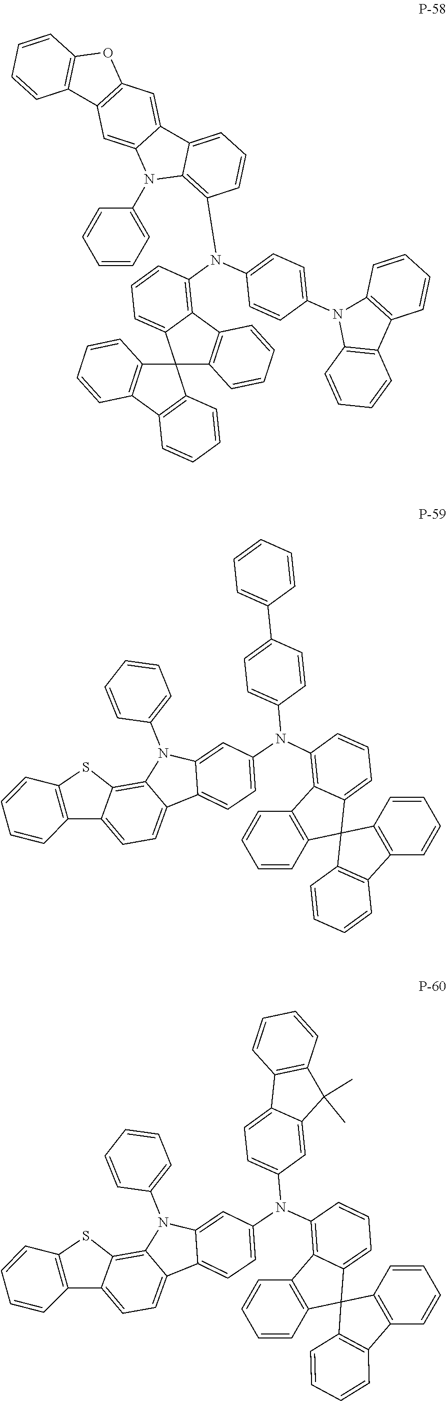 [SMILES: c1ccc(-n2c3cc4c(cc3c3cccc(N(c5ccc(-n6c7ccccc7c7ccccc76)cc5)c5cccc6c5-c5ccccc5C65c6ccccc6-c6ccccc65)c32)oc2ccccc24)cc1 ; c1ccc(-c2ccc(N(c3ccc4c5ccc6c7ccccc7sc6c5n(-c5ccccc5)c4c3)c3cccc4c3-c3ccccc3C43c4ccccc4-c4ccccc43)cc2)cc1 ; CC1(C)c2ccccc2-c2ccc(N(c3ccc4c5ccc6c7ccccc7sc6c5n(-c5ccccc5)c4c3)c3cccc4c3-c3ccccc3C43c4ccccc4-c4ccccc43)cc21]

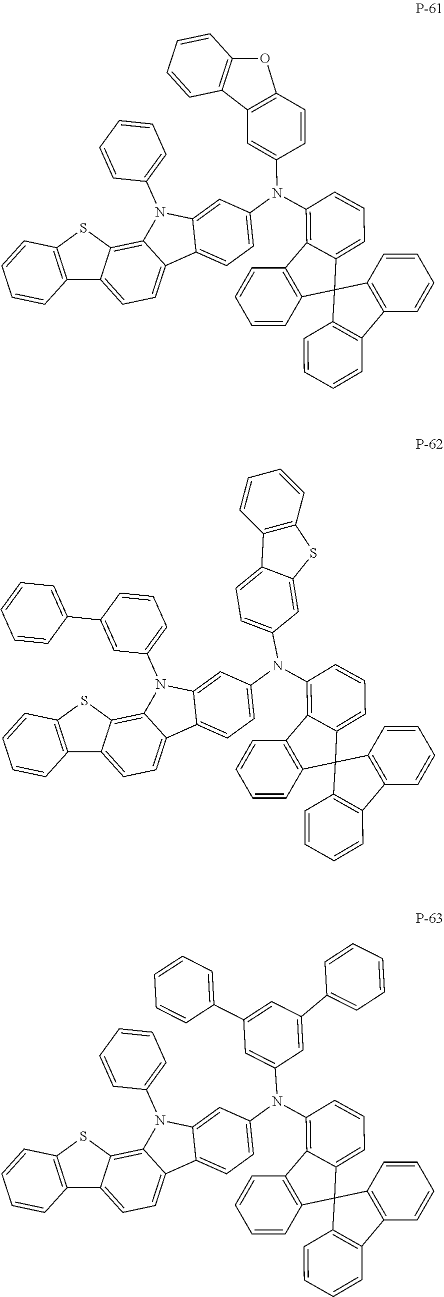 [SMILES: c1ccc(-n2c3cc(N(c4ccc5oc6ccccc6c5c4)c4cccc5c4-c4ccccc4C54c5ccccc5-c5ccccc54)ccc3c3ccc4c5ccccc5sc4c32)cc1 ; c1ccc(-c2cccc(-n3c4cc(N(c5ccc6c(c5)sc5ccccc56)c5cccc6c5-c5ccccc5C65c6ccccc6-c6ccccc65)ccc4c4ccc5c6ccccc6sc5c43)c2)cc1 ; c1ccc(-c2cc(-c3ccccc3)cc(N(c3ccc4c5ccc6c7ccccc7sc6c5n(-c5ccccc5)c4c3)c3cccc4c3-c3ccccc3C43c4ccccc4-c4ccccc43)c2)cc1]

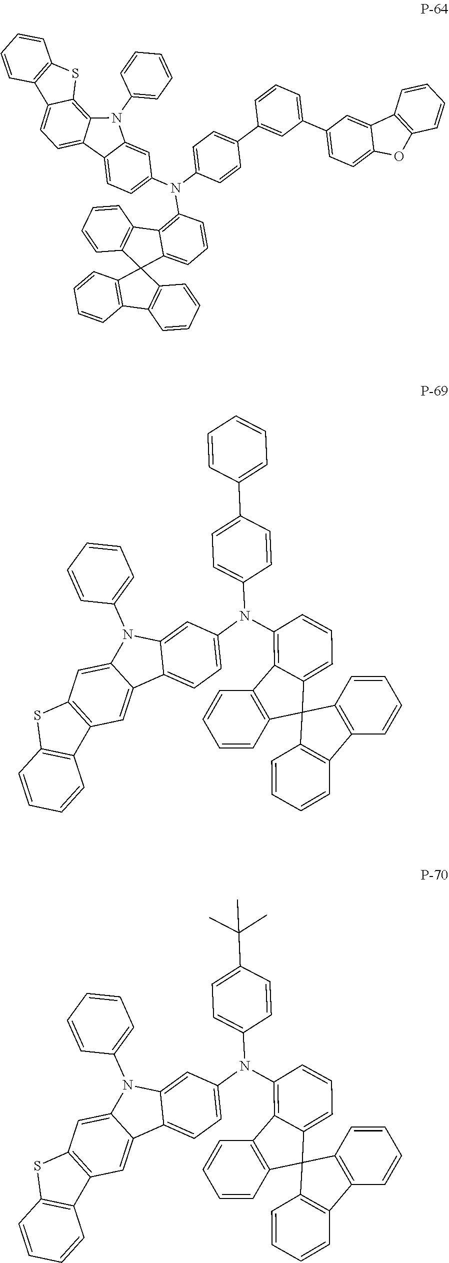 [SMILES: c1ccc(-n2c3cc(N(c4ccc(-c5cccc(-c6ccc7oc8ccccc8c7c6)c5)cc4)c4cccc5c4-c4ccccc4C54c5ccccc5-c5ccccc54)ccc3c3ccc4c5ccccc5sc4c32)cc1 ; c1ccc(-c2ccc(N(c3ccc4c5cc6c(cc5n(-c5ccccc5)c4c3)sc3ccccc36)c3cccc4c3-c3ccccc3C43c4ccccc4-c4ccccc43)cc2)cc1 ; CC(C)(C)c1ccc(N(c2ccc3c4cc5c(cc4n(-c4ccccc4)c3c2)sc2ccccc25)c2cccc3c2-c2ccccc2C32c3ccccc3-c3ccccc32)cc1]

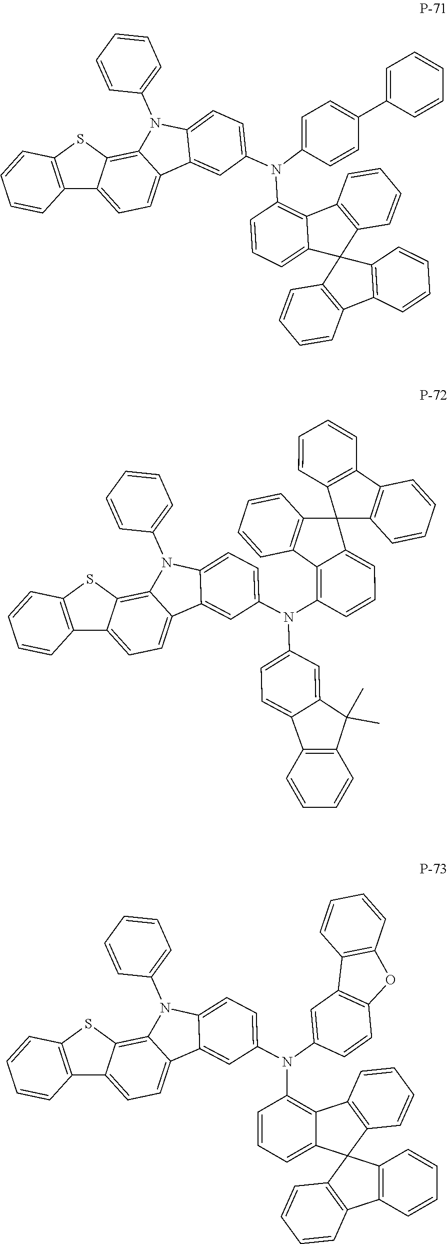 [SMILES: c1ccc(-c2ccc(N(c3ccc4c(c3)c3ccc5c6ccccc6sc5c3n4-c3ccccc3)c3cccc4c3-c3ccccc3C43c4ccccc4-c4ccccc43)cc2)cc1 ; CC1(C)c2ccccc2-c2ccc(N(c3ccc4c(c3)c3ccc5c6ccccc6sc5c3n4-c3ccccc3)c3cccc4c3-c3ccccc3C43c4ccccc4-c4ccccc43)cc21 ; c1ccc(-n2c3ccc(N(c4ccc5oc6ccccc6c5c4)c4cccc5c4-c4ccccc4C54c5ccccc5-c5ccccc54)cc3c3ccc4c5ccccc5sc4c32)cc1]

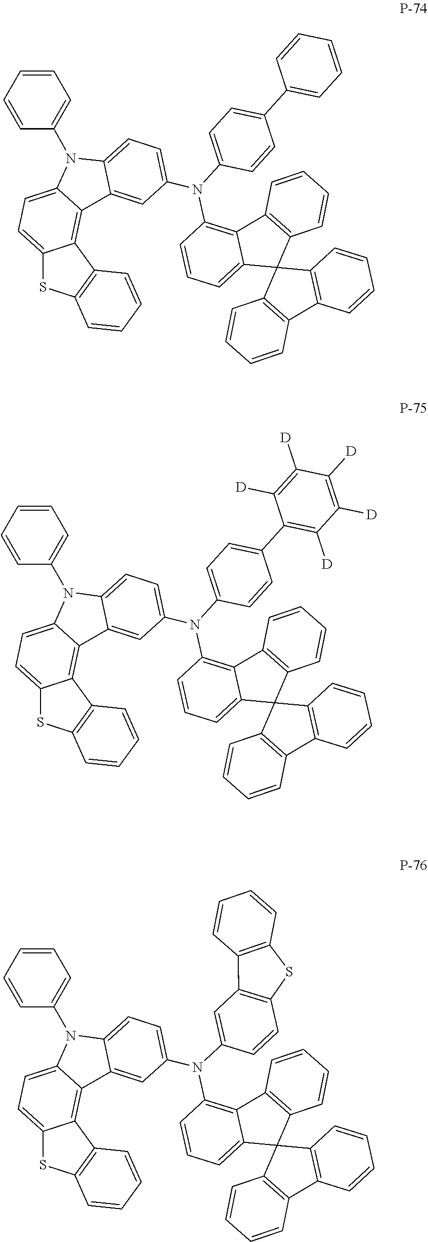 [SMILES: c1ccc(-c2ccc(N(c3ccc4c(c3)c3c5c(ccc3n4-c3ccccc3)sc3ccccc35)c3cccc4c3-c3ccccc3C43c4ccccc4-c4ccccc43)cc2)cc1 ; [2H]c1c([2H])c([2H])c(-c2ccc(N(c3ccc4c(c3)c3c5c(ccc3n4-c3ccccc3)sc3ccccc35)c3cccc4c3-c3ccccc3C43c4ccccc4-c4ccccc43)cc2)c([2H])c1[2H] ; c1ccc(-n2c3ccc(N(c4ccc5sc6ccccc6c5c4)c4cccc5c4-c4ccccc4C54c5ccccc5-c5ccccc54)cc3c3c4c(ccc32)sc2ccccc24)cc1]

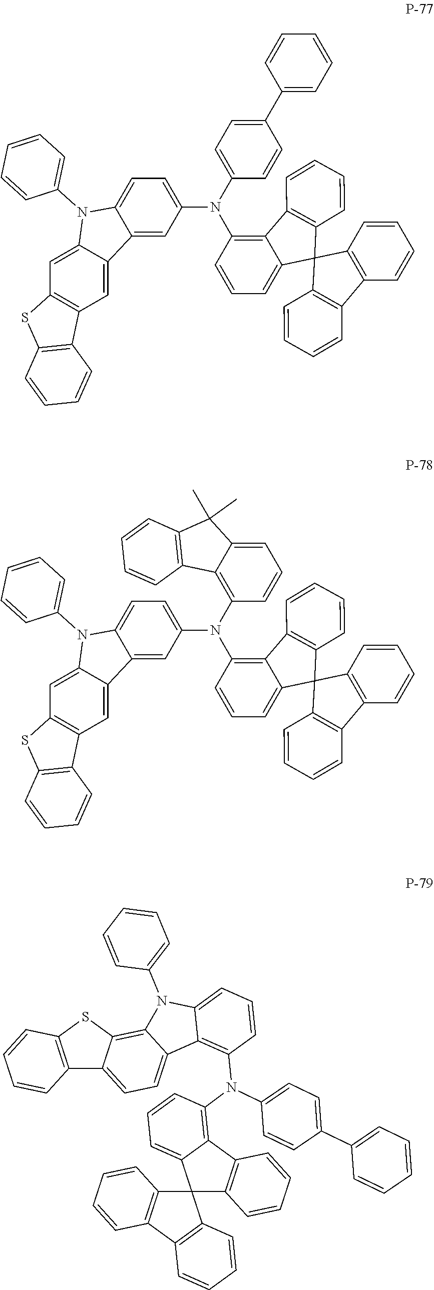 [SMILES: c1ccc(-c2ccc(N(c3ccc4c(c3)c3cc5c(cc3n4-c3ccccc3)sc3ccccc35)c3cccc4c3-c3ccccc3C43c4ccccc4-c4ccccc43)cc2)cc1 ; CC1(C)c2ccccc2-c2c(N(c3ccc4c(c3)c3cc5c(cc3n4-c3ccccc3)sc3ccccc35)c3cccc4c3-c3ccccc3C43c4ccccc4-c4ccccc43)cccc21 ; c1ccc(-c2ccc(N(c3cccc4c3-c3ccccc3C43c4ccccc4-c4ccccc43)c3cccc4c3c3ccc5c6ccccc6sc5c3n4-c3ccccc3)cc2)cc1]

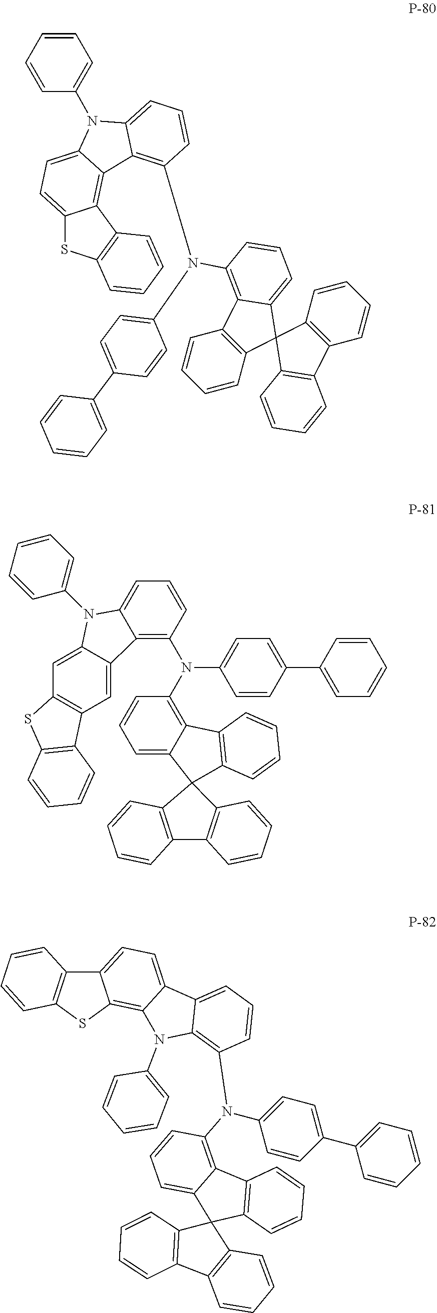 [SMILES: c1ccc(-c2ccc(N(c3cccc4c3-c3ccccc3C43c4ccccc4-c4ccccc43)c3cccc4c3c3c5c(ccc3n4-c3ccccc3)sc3ccccc35)cc2)cc1 ; c1ccc(-c2ccc(N(c3cccc4c3-c3ccccc3C43c4ccccc4-c4ccccc43)c3cccc4c3c3cc5c(cc3n4-c3ccccc3)sc3ccccc35)cc2)cc1 ; c1ccc(-c2ccc(N(c3cccc4c3-c3ccccc3C43c4ccccc4-c4ccccc43)c3cccc4c5ccc6c7ccccc7sc6c5n(-c5ccccc5)c34)cc2)cc1]

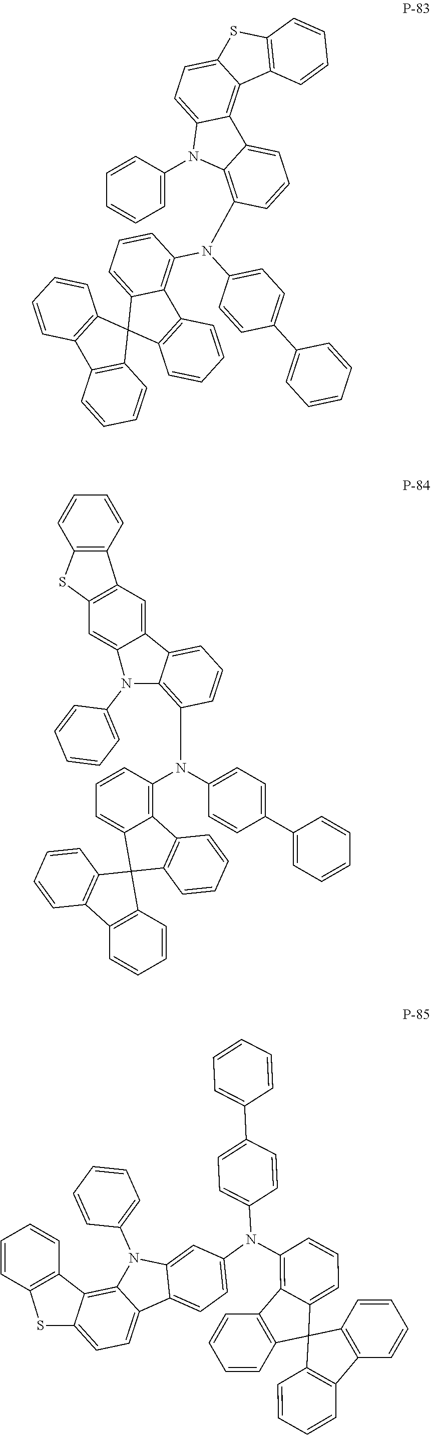 [SMILES: c1ccc(-c2ccc(N(c3cccc4c3-c3ccccc3C43c4ccccc4-c4ccccc43)c3cccc4c5c6c(ccc5n(-c5ccccc5)c34)sc3ccccc36)cc2)cc1 ; c1ccc(-c2ccc(N(c3cccc4c3-c3ccccc3C43c4ccccc4-c4ccccc43)c3cccc4c5cc6c(cc5n(-c5ccccc5)c34)sc3ccccc36)cc2)cc1 ; c1ccc(-c2ccc(N(c3ccc4c5ccc6sc7ccccc7c6c5n(-c5ccccc5)c4c3)c3cccc4c3-c3ccccc3C43c4ccccc4-c4ccccc43)cc2)cc1]

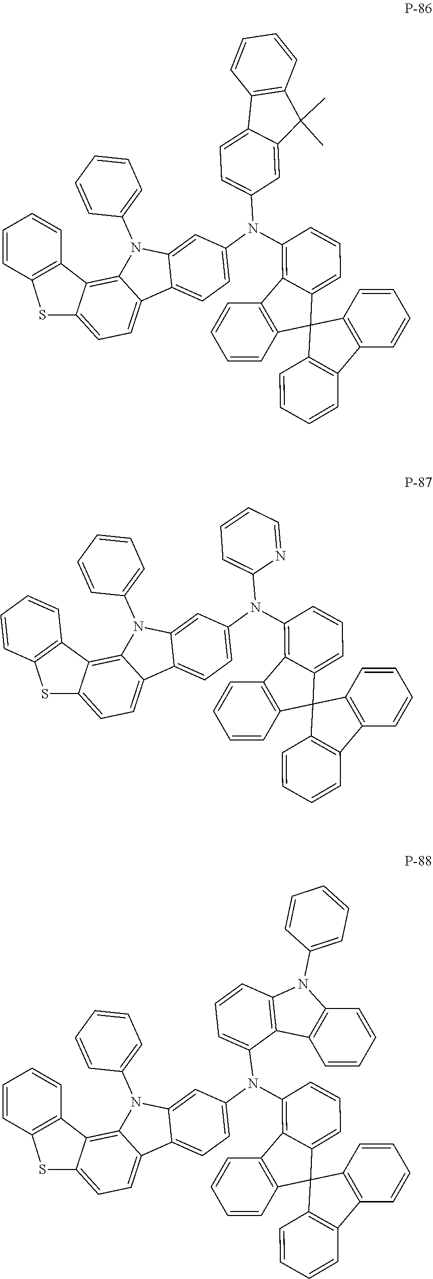 [SMILES: CC1(C)c2ccccc2-c2ccc(N(c3ccc4c5ccc6sc7ccccc7c6c5n(-c5ccccc5)c4c3)c3cccc4c3-c3ccccc3C43c4ccccc4-c4ccccc43)cc21 ; c1ccc(-n2c3cc(N(c4ccccn4)c4cccc5c4-c4ccccc4C54c5ccccc5-c5ccccc54)ccc3c3ccc4sc5ccccc5c4c32)cc1 ; c1ccc(-n2c3ccccc3c3c(N(c4ccc5c6ccc7sc8ccccc8c7c6n(-c6ccccc6)c5c4)c4cccc5c4-c4ccccc4C54c5ccccc5-c5ccccc54)cccc32)cc1]

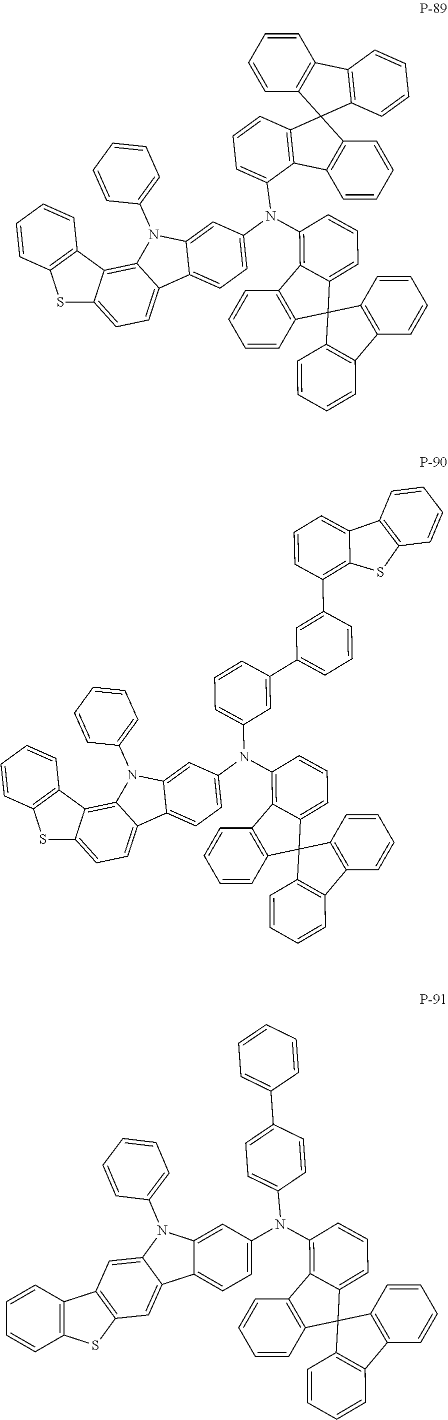 [SMILES: c1ccc(-n2c3cc(N(c4cccc5c4-c4ccccc4C54c5ccccc5-c5ccccc54)c4cccc5c4-c4ccccc4C54c5ccccc5-c5ccccc54)ccc3c3ccc4sc5ccccc5c4c32)cc1 ; c1ccc(-n2c3cc(N(c4cccc(-c5cccc(-c6cccc7c6sc6ccccc67)c5)c4)c4cccc5c4-c4ccccc4C54c5ccccc5-c5ccccc54)ccc3c3ccc4sc5ccccc5c4c32)cc1 ; c1ccc(-c2ccc(N(c3ccc4c5cc6sc7ccccc7c6cc5n(-c5ccccc5)c4c3)c3cccc4c3-c3ccccc3C43c4ccccc4-c4ccccc43)cc2)cc1]

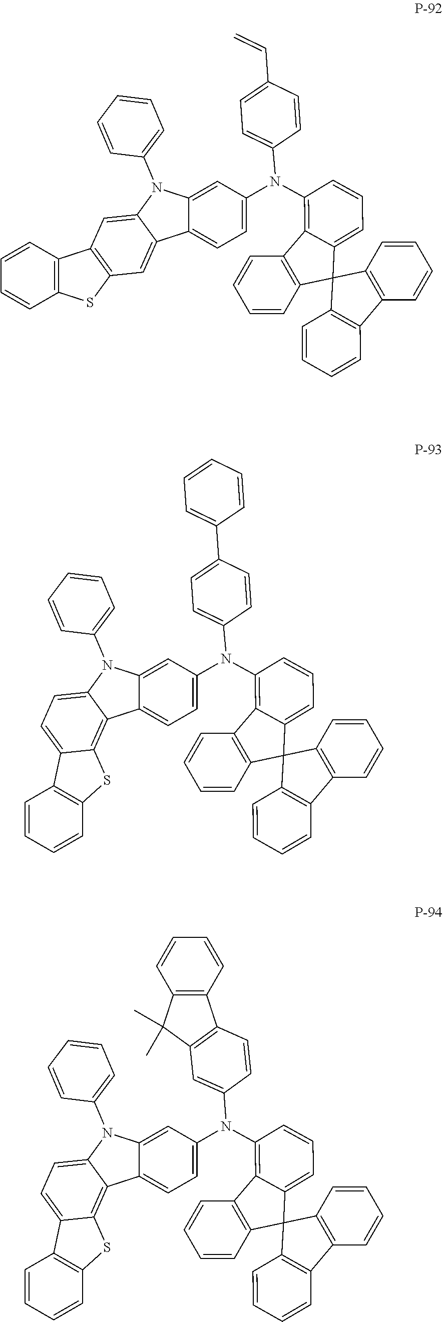 [SMILES: C=Cc1ccc(N(c2ccc3c4cc5sc6ccccc6c5cc4n(-c4ccccc4)c3c2)c2cccc3c2-c2ccccc2C32c3ccccc3-c3ccccc32)cc1 ; c1ccc(-c2ccc(N(c3ccc4c5c6sc7ccccc7c6ccc5n(-c5ccccc5)c4c3)c3cccc4c3-c3ccccc3C43c4ccccc4-c4ccccc43)cc2)cc1 ; CC1(C)c2ccccc2-c2ccc(N(c3ccc4c5c6sc7ccccc7c6ccc5n(-c5ccccc5)c4c3)c3cccc4c3-c3ccccc3C43c4ccccc4-c4ccccc43)cc21]

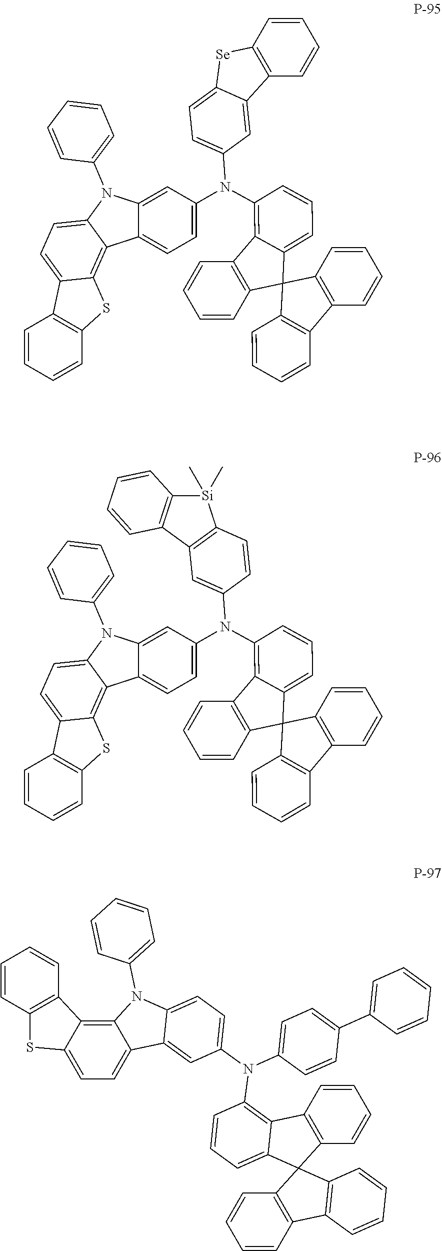 [SMILES: c1ccc(-n2c3cc(N(c4ccc5[se]c6ccccc6c5c4)c4cccc5c4-c4ccccc4C54c5ccccc5-c5ccccc54)ccc3c3c4sc5ccccc5c4ccc32)cc1 ; C[Si]1(C)c2ccccc2-c2cc(N(c3ccc4c5c6sc7ccccc7c6ccc5n(-c5ccccc5)c4c3)c3cccc4c3-c3ccccc3C43c4ccccc4-c4ccccc43)ccc21 ; c1ccc(-c2ccc(N(c3ccc4c(c3)c3ccc5sc6ccccc6c5c3n4-c3ccccc3)c3cccc4c3-c3ccccc3C43c4ccccc4-c4ccccc43)cc2)cc1]

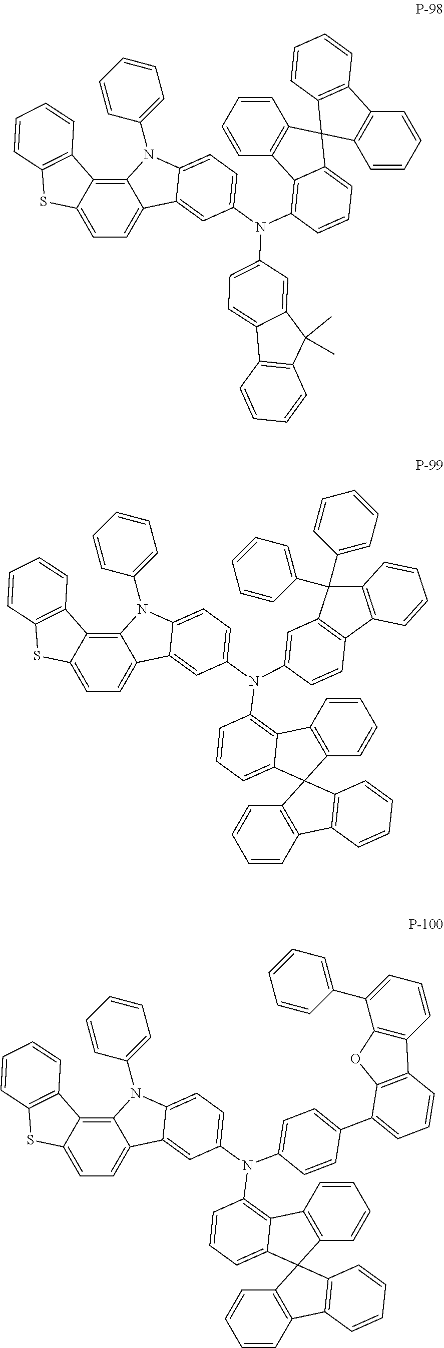 [SMILES: CC1(C)c2ccccc2-c2ccc(N(c3ccc4c(c3)c3ccc5sc6ccccc6c5c3n4-c3ccccc3)c3cccc4c3-c3ccccc3C43c4ccccc4-c4ccccc43)cc21 ; c1ccc(-n2c3ccc(N(c4ccc5c(c4)C(c4ccccc4)(c4ccccc4)c4ccccc4-5)c4cccc5c4-c4ccccc4C54c5ccccc5-c5ccccc54)cc3c3ccc4sc5ccccc5c4c32)cc1 ; c1ccc(-c2cccc3c2oc2c(-c4ccc(N(c5ccc6c(c5)c5ccc7sc8ccccc8c7c5n6-c5ccccc5)c5cccc6c5-c5ccccc5C65c6ccccc6-c6ccccc65)cc4)cccc23)cc1]

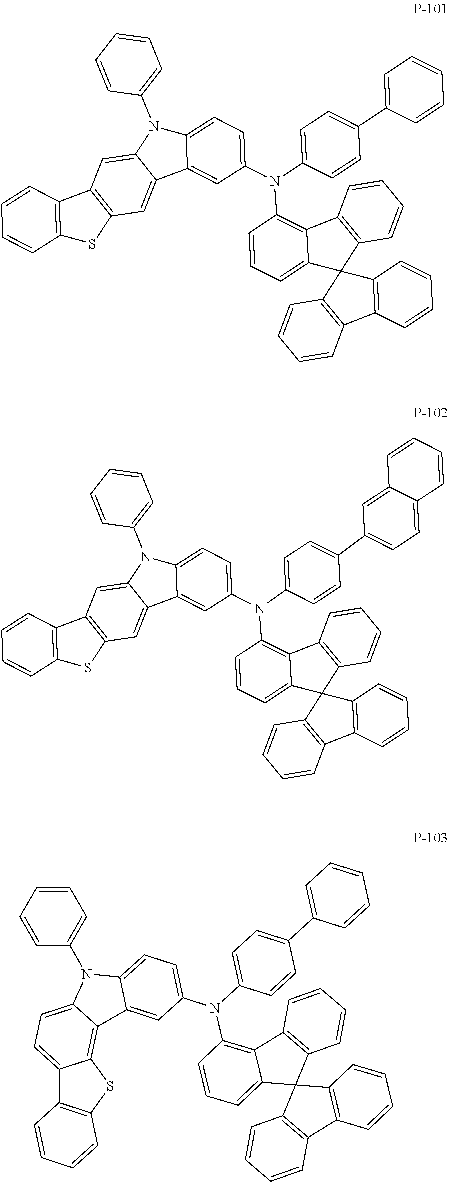 [SMILES: c1ccc(-c2ccc(N(c3ccc4c(c3)c3cc5sc6ccccc6c5cc3n4-c3ccccc3)c3cccc4c3-c3ccccc3C43c4ccccc4-c4ccccc43)cc2)cc1 ; c1ccc(-n2c3ccc(N(c4ccc(-c5ccc6ccccc6c5)cc4)c4cccc5c4-c4ccccc4C54c5ccccc5-c5ccccc54)cc3c3cc4sc5ccccc5c4cc32)cc1 ; c1ccc(-c2ccc(N(c3ccc4c(c3)c3c5sc6ccccc6c5ccc3n4-c3ccccc3)c3cccc4c3-c3ccccc3C43c4ccccc4-c4ccccc43)cc2)cc1]

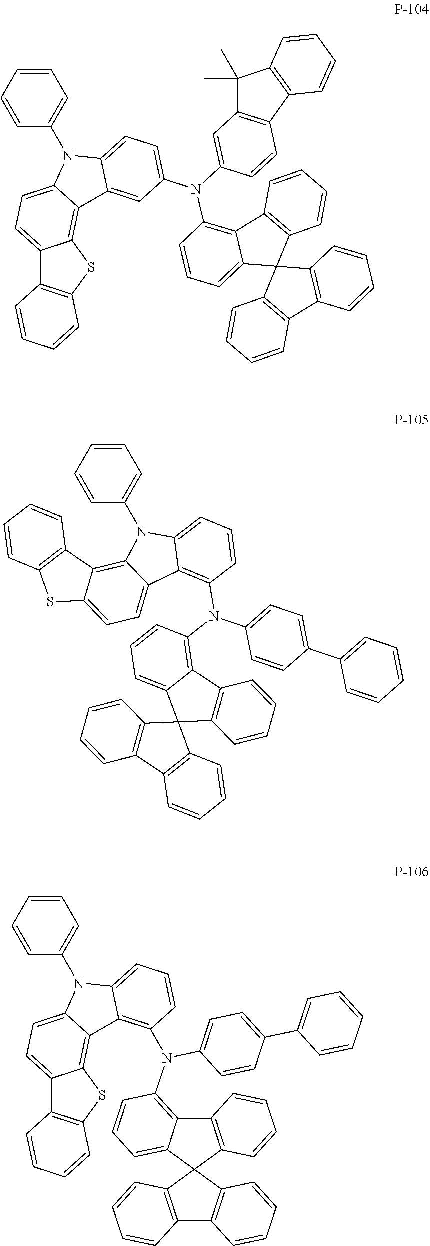 [SMILES: CC1(C)c2ccccc2-c2ccc(N(c3ccc4c(c3)c3c5sc6ccccc6c5ccc3n4-c3ccccc3)c3cccc4c3-c3ccccc3C43c4ccccc4-c4ccccc43)cc21 ; c1ccc(-c2ccc(N(c3cccc4c3-c3ccccc3C43c4ccccc4-c4ccccc43)c3cccc4c3c3ccc5sc6ccccc6c5c3n4-c3ccccc3)cc2)cc1 ; c1ccc(-c2ccc(N(c3cccc4c3-c3ccccc3C43c4ccccc4-c4ccccc43)c3cccc4c3c3c5sc6ccccc6c5ccc3n4-c3ccccc3)cc2)cc1]

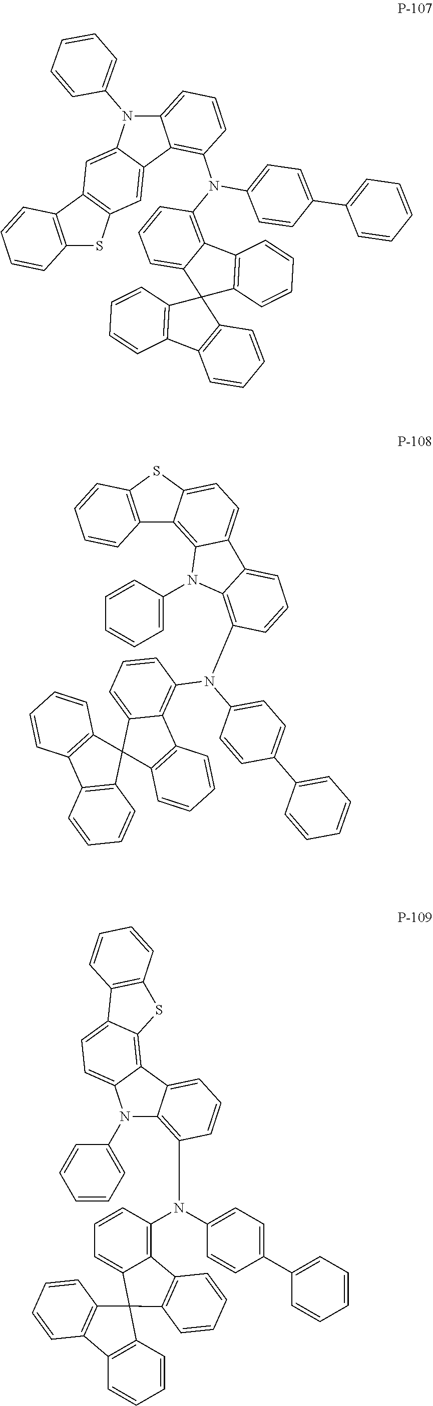 [SMILES: c1ccc(-c2ccc(N(c3cccc4c3-c3ccccc3C43c4ccccc4-c4ccccc43)c3cccc4c3c3cc5sc6ccccc6c5cc3n4-c3ccccc3)cc2)cc1 ; c1ccc(-c2ccc(N(c3cccc4c3-c3ccccc3C43c4ccccc4-c4ccccc43)c3cccc4c5ccc6sc7ccccc7c6c5n(-c5ccccc5)c34)cc2)cc1 ; c1ccc(-c2ccc(N(c3cccc4c3-c3ccccc3C43c4ccccc4-c4ccccc43)c3cccc4c5c6sc7ccccc7c6ccc5n(-c5ccccc5)c34)cc2)cc1]

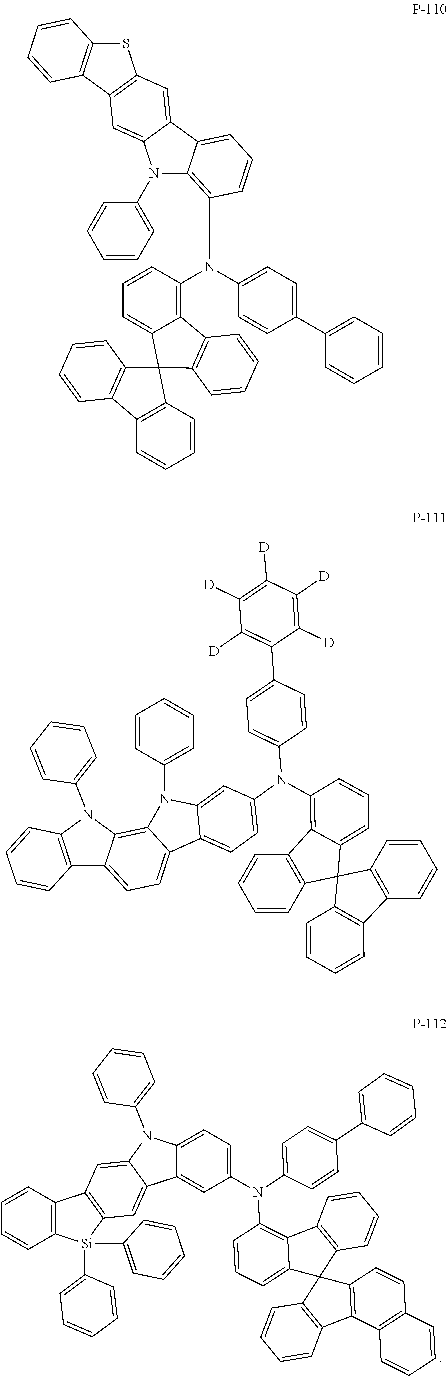 [SMILES: c1ccc(-c2ccc(N(c3cccc4c3-c3ccccc3C43c4ccccc4-c4ccccc43)c3cccc4c5cc6sc7ccccc7c6cc5n(-c5ccccc5)c34)cc2)cc1 ; [2H]c1c([2H])c([2H])c(-c2ccc(N(c3ccc4c5ccc6c7ccccc7n(-c7ccccc7)c6c5n(-c5ccccc5)c4c3)c3cccc4c3-c3ccccc3C43c4ccccc4-c4ccccc43)cc2)c([2H])c1[2H] ; c1ccc(-c2ccc(N(c3ccc4c(c3)c3cc5c(cc3n4-c3ccccc3)-c3ccccc3[Si]5(c3ccccc3)c3ccccc3)c3cccc4c3-c3ccccc3C43c4ccccc4-c4c3ccc3ccccc43)cc2)cc1]

4. An organic electric element comprising:
a first electrode;
a second electrode; and
an organic material layer positioned between the first electrode and the second electrode,
wherein the organic material layer contains the compound of claim 1.

5. The organic electric element of claim 4, wherein the compound is contained in at least one layer of a hole injection layer, a hole transport layer, a light emitting auxiliary layer, a light emitting layer, an electron auxiliary layer, an electron transport layer, and an electron injection layer of the organic material layer, and the compound is contained as a single kind of compound alone or a mixture of two or more kinds of compounds.

6. The organic electric element of claim 4, wherein the compound is used for a hole injection layer or a light emitting auxiliary layer.

7. The organic electric element of claim 4, further comprising a light efficiency improving layer formed on one surface of at least one of the first and second electrodes, the surface being the opposite side to the organic material layer.

8. The organic electric element of claim 5, wherein the organic material layer is formed by a spin coating process, a nozzle printing process, an inkjet printing process, a slot coating process, a dip coating process, or a roll-to-roll process.

9. An electronic device comprising:
a display device comprising the organic electric element of claim 4; and
a controller for driving the display device.

10. The electronic device of claim 9, wherein the organic electric element is one of an organic light emitting diode, an organic solar cell, an organic photo conductor, an organic transistor, and an element for a monochromatic or white illumination.
